(19) United States
(12) Patent Application Publication
(10) Pub. No.: US 2012/0108819 A1
(43) Pub. Date: May 3, 2012

(54) N-Heterocyclic Carbene Complexes, Their Preparation And Use

(75) Inventors: A. Stephen K. Hashmi, Stuttgart (DE); Christian Lothschütz, Schriesheim (DE)

(73) Assignee: BASF SE, Ludwigshafen (DE)

(21) Appl. No.: 13/284,201

(22) Filed: Oct. 28, 2011

Related U.S. Application Data
(60) Provisional application No. 61/407,485, filed on Oct. 28, 2010.

(51) Int. Cl. C07F 15/00; C07C 1/32; C07F 1/12

(52) U.S. Cl. 548/103; 556/112; 558/302; 585/469

(57) ABSTRACT

Described are N-heterocyclic carbene complexes of the formula I,

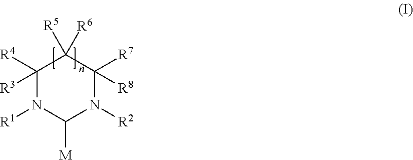

where n is 0 or 1, M is a metal atom containing group, R¹ is selected from hydrogen alkyl, cycloalkyl, heterocycloalkyl, aryl and hetaryl, R² is selected from hydrogen, alkyl, cycloalkyl, heterocycloalkyl, aryl and hetaryl, wherein R¹ and R² do not both stand for hydrogen.

Furthermore, also described are methods for their preparation and their use as catalysts employed in a C—C, C—O, C—N or C—H bond formation reaction.

### CROSS-REFERENCE TO RELATED APPLICATIONS

This application claims priority under 35 U.S.C. §119(e) to U.S. Provisional Application No. 61/407,485, filed Oct. 28, 2010, the disclosure of which is incorporated herein by reference in its entirety.

### FIELD OF THE INVENTION

Embodiments of the present invention relate to N-heterocyclic carbene complexes, a method for their preparation and their use as catalysts.

### BACKGROUND

In 1968 H. W. Wanzlick and K. Öfele independently reported the first direct synthesis of metal complexes with N-heterocyclic carbenes (NHCs) as ligands. At that time, no one would have been able to predict the role these ligands would play a few decades later. Especially the NHC complexes of Pd(II) find broad application in modern organic and organometallic chemistry. The advantages of NHCs like thermal stability, low toxicity, insensitivity against oxygen over other ligands like phosphanes have allowed the application of NHC complexes in a number of different coupling reactions, e.g. Heck, Sonogashira, Suzuki-Miyaura, Stille, Hartwig-Buchwald coupling, and others. In particular, the Suzuki-Miyaura reaction today plays an important role due to the stability, availability and low toxicity of the employed boronic acids.

In addition to the use in catalysis, NHC complexes are also useful in many other fields like medical applications, nanoparticles, supramolecular chemistry, self-assembly, photochemistry, liquid crystals, polymerization and electronically active materials.

Besides the great number of advantages of these special ligands, the synthesis of saturated NHC complexes remains challenging. A. J. Arduengo, R. Krafczyk and R. Schmutzler describe in Tetrahedron 1999, 55, 14523-14534 the synthesis of imidazolylidenes, imidazolinylidenes and imidazolidines starting from glyoxal via the corresponding diimines and diamine dihydrochlorides. Subsequently, the diamine dihydrochlorides are converted into the corresponding imidazolinium salts by reaction with an ortho formate as C₁ building block. Reduction of the imidazolinium salts with lithium aluminium hydride leads to imidazolidines, whereas deprotonation with potassium hydride leads to the corresponding imidazolin-2-ylidenes. Nevertheless, this methodology does in particular not allow the formation of unsymmetrically substituted NHCs.

B. A. Bhanu Prasad and S. R. Gilbertson describe in Org. Lett. 2009, 11, 3710-3713 a one-pot synthesis of unsymmetrical NHC ligands from N-(2-iodoethyl)arylamine salts. This route is limited to the formation of NHCs with certain substituents.

K. Bartel and W. P. Fehlhammer describe in Angew. Chem. Int. Ed. 1974, 13, 599-600 the formation of oxazolidinylidene and perhydrooxazinylidene complexes of Pd, Pt and Au by reaction of 2-, and 3-hydroxyalkyl isocyanides with metal compounds.

U. Plaia and W. P. Fehlhammer describe in J. Am. Chem. Soc. 1985, 107, 2171-2172 the preparation of hexakis(oxazolidin-2-ylidene)cobalt(III) and -rhodium(III).

M. Tamm and F. E. Hahn describe in Coord. Chem. Rev. 1999, 182, 175-209 the formation of carben complexes from coordinated β-functional phenyl isocyanides.

R. A. Michelin, L. Zanotto, D. Braga, P. Sabatino and R. J. Angelici describe in Inorg. Chem. 1988, 27, 85-92 the synthesis of cyclic aminooxycarbene complexes of Pt(II) via a cyclization reaction of isocyanide complexes with 2-bromoethanol.

I. Yu, C. J. Wallis, B. O. Patrick, P. L. Diaconescu and P. Mehrkhodavandi in doi:10.1021/om100841j reported a remotely related reaction, using a primary amine and a strain-activated isonitrile/phosphane chelate on iron.

R. A. Michelin, L. Zanotto, D. Braga, P. Sabatino and R. J. Angelici describe in Inorg. Chem. 1988, 27, 93-99 the synthesis of cyclic diaminocarbene complexes of Pd(II) and Pt(II) via a cyclization reaction of isocyanide complexes with 2-bromoethylamine.

R. A. Michelin, A. J. L. Pombeiro and M. F. C. Guedes da Silva report in Coord. Chem. Rev. 2001, 218, 75-112 on aminocarbene complexes derived from nucleophilic addition to isocyanide ligands.

The method described in the three last-mentioned documents delivers NHCs that are unsubstituted at one nitrogen atom and only alkyl substituents could be attached to this nitrogen atom subsequently. The synthesis of the free NHC often requires long organic-synthetic steps and the attachment of the thus synthesized NHC ligand to the metal is often difficult and requires additional synthetic effort.

There is still a great need for an effective method that allows the preparation N,N′-disubstituted NHC-complexes, and in particular unsymmetrically disubstituted NHC-complexes. The metal-NHC-complexes should be air-stable and easy to handle.

### SUMMARY OF THE INVENTION

One or more embodiments of the present invention relate to a process for preparing compounds of the general formula (I)

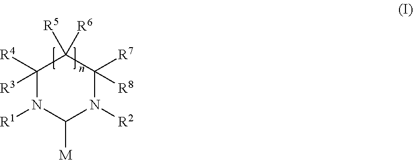

where
n is 0 or 1,
M is a metal atom containing group,
R¹ is selected from hydrogen, alkyl, cycloalkyl, heterocycloalkyl, aryl and hetaryl,
R² is selected from hydrogen, alkyl, cycloalkyl, heterocycloalkyl, aryl and hetaryl, wherein R¹ and R² do not both stand for hydrogen,
R³ and R⁴ are independently selected from hydrogen and in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
or R³ and R⁴ together with the carbon atom to which they are bound are C═O,
or R³ is a group O—R^{3a} and
   for n=0 R⁴ and R⁷ stand for the bond equivalent of a double bond between the carbon atoms carrying R⁴ and R⁷, respectively or
   for n=1 R⁴ and R⁵ stand for the bond equivalent of a double bond between the carbon atoms carrying R⁴ and R⁵, respectively, where
   R^{3a} is a group bound to the oxygen via a carbon atom, silicon atom, sulfur atom, phosphorus atom, boron atom or titanium atom,
R⁵, R⁶, R⁷ and R⁸ are independently selected from hydrogen and in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
   wherein the two radicals R² and R⁸ may also form together with the N atom to which R² is bound a 3- to 12-membered, unsubstituted or substituted nitrogen heterocycle which may optionally have 1, 2 or 3 further heteroatoms or heteroatom containing groups independently selected from O, N, NR^{a} and S as ring members, wherein R^{a} is hydrogen, alkyl, cycloalkyl or aryl,
   or
   wherein if n=0, R⁴ and R⁷ also may stand for the bond equivalent of a double bond between the carbon atoms carrying R⁴ and R⁷.

According to one or more embodiments, the process comprises:
a1) the reaction of an isonitrile complex of the general formula (II)

R¹—N≡C-M (II)
in which R¹ and M have one of the meanings given above,
with a compound of the general formulae (III) or (IIIa)

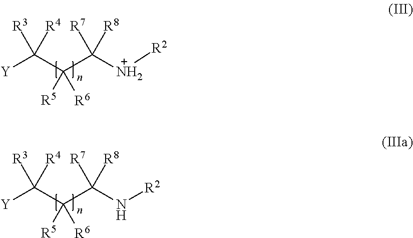

in which
n, R², R³, R⁴, R⁵, R⁶, R⁷ and R⁸ have one of the meanings given above,
X⁻ is an anion equivalent, and
Y is a leaving group, or
   if R³ and R⁴ together with the carbon atom to which they are bound are C═O then Y is a group O—Y^{a}, where Y^{a} is unsubstituted or substituted alkyl, unsubstituted or substituted aryl, unsubstituted or substituted arylcarbonyl or unsubstituted or substituted alkyl carbonyl, and
b1) optionally, if R³ and R⁴ together with the carbon atom to which they are bound are C═O, subjecting the product obtained in step a1) to a further reaction with a compound R^{3a}—Z, where Z is a leaving group, in the presence of a base to obtain a compound of the formula (I) where R³ is a group O—R^{3a} and for n=0 R⁴ and R⁷ stand for the bond equivalent of a double bond between the carbon atoms bound to R⁴ and R⁷ or for n=1 R⁴ and R⁵ stand for the bond equivalent of a double bond between the carbon atoms bound to R⁴ and R⁵,

In other embodiments, the process comprises:
a2) the reaction of an isonitrile complex of the general formula II

R¹—N≡C-M (II)
where R¹ and M have one of the meanings given above,
with a compound of the general formula (V)

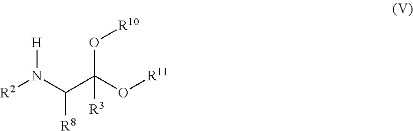

where
R², R³ and R⁸ have one of the meanings given above; and
R¹⁰ and R¹¹ are independently selected from C₁-C₄-alkyl or R¹⁰ and R¹¹ together are linear C₂-C₄-alkylene which may be substituted by one or more C₁-C₄ alkyl radicals;
to give an intermediate compound of the formula (VI)

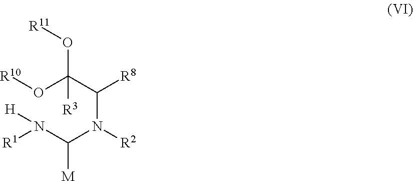

and
b2) the treatment of the intermediate compound of the formula (VI) with an acid, wherein in compound (I) obtained according to this variant n is 0, and R⁴ and R⁷ stand for the bond equivalent of a double bond between the carbon atoms carrying R⁴ and R⁷.

Other embodiments provide the process comprises:
a3) the reaction of an isonitrile complex of the general formula II

R¹—N≡C-M (II)
where R¹ and M have one of the meanings given above,
with a compound of the general formulae (IIIb) or (IIIc)

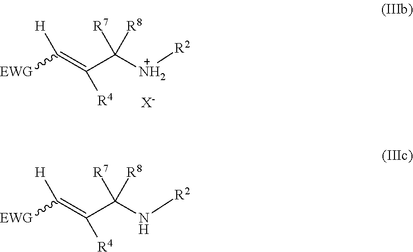

where
R², R⁴, R⁷ and R⁸ have one of the meanings given above;
X⁻ is an anion equivalent; and
EWG is (C(O)R¹⁴, C(O)OR¹⁴, NO₂, S(O)R¹⁴ or S(O)₂R¹⁴, where R¹⁴ is hydrogen, alkyl, cycloalkyl or aryl;
wherein in compound (I) obtained according to variant a3) n is 0 and R³ is CH₂-EWG.

In a first aspect, the invention provides a process for preparing compounds of the general formula (I)

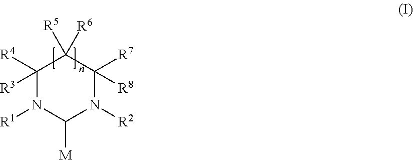

where
n is =0 or 1,
M is a metal atom containing group,
R¹ is selected from hydrogen, alkyl, cycloalkyl, heterocycloalkyl, aryl and hetaryl,
R² is selected from hydrogen, alkyl, cycloalkyl, heterocycloalkyl, aryl and hetaryl, wherein R¹ and R² do not both stand for hydrogen,
R³ and R⁴ are independently selected from hydrogen and in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
   or R³ and R⁴ together with the carbon atom to which they are bound are C═O,
   or R³ is a group O—R^{3a} and for n=0, R⁴ and R⁷ stand for the bond equivalent of a double bond between the carbon atoms carrying R⁴ and R⁷, respectively
   or R³ is a group O—R^{3a} and for n=1, R⁴ and R⁵ stand for the bond equivalent of a double bond between the carbon atoms carrying R⁴ and R⁵, respectively, where R^{3a} is a group bound to the oxygen via a carbon, silicon, sulfur, phosphorus, boron or titanium atom,
R⁵, R⁶, R⁷ and R⁸ are independently selected from hydrogen and in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
wherein the two radicals R² and R⁸ may also form together with the N atom to which R² is bound and the carbon atom to which R⁸ is bound a 3- to 12-membered, unsubstituted or substituted nitrogen heterocycle which may optionally have 1, 2 or 3 further heteroatoms or heteroatom containing groups independently selected from O, N, NR^{a} and S as ring members, wherein R^{a} is hydrogen, alkyl, cycloalkyl or aryl,

According to embodiments of this aspect, the process comprises:
a1) the reaction of an isonitrile complex of the general formula (II)

R¹—N≡C-M (II)
in which R¹ and M have one of the meanings given above,
with a compound of the general formulae (III) or (IIIa)

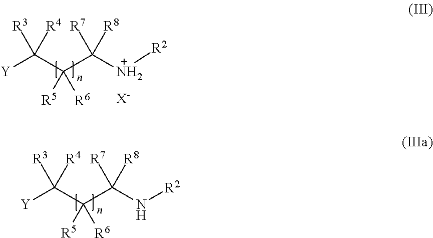

in which
n, R², R³, R⁴, R⁵, R⁶, R⁷ and R⁸ have one of the meanings given above,
X⁻ is an anion equivalent, and
Y is a leaving group, or
   if R³ and R⁴ together with the carbon atom to which they are bound are C═O then Y is a group O—Y^{a}, where Y^{a} is unsubstituted or substituted alkyl, unsubstituted or substituted aryl, unsubstituted or substituted arylcarbonyl or unsubstituted or substituted alkyl carbonyl, and
b1) optionally, if R³ and R⁴ together with the carbon atom to which they are bound are C═O, subjecting the product obtained in step a1) to a further reaction with a compound R^{3a}—Z, where Z is a leaving group, in the presence of a base to obtain a compound of the formula (I) where R³ is a group O—R^{3a} and for n=0 R⁴ and R⁷ stand for the bond equivalent of a double bond between the carbon atoms bound to R⁴ and R⁷ or for n=1 R⁴ and R⁵ stand for the bond equivalent of a double bond between the carbon atoms bound to R⁴ and R⁵.

According to a particular embodiment, the process of the invention is used for the formation of unsymmetrically substituted compounds of the formula (I). According to certain embodiments, R¹ and R² have different meanings.

In one or more embodiments, provided is a process for preparing compounds of the formula (I-A.1) or (I-A.2)

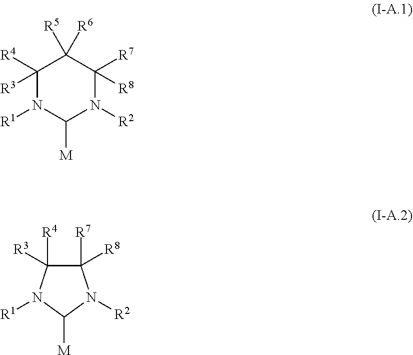

where
M, R¹, R², R⁵, R⁶, R⁷ and R⁸ have a meaning as defined above and in the following,
R³ and R⁴ are independently selected from hydrogen and in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino.

In certain embodiments, the process comprises:

a1) the reaction of an isonitrile complex of the general formula (II)

R¹—N≡C-M (II)
in which R¹ and M have one of the meanings defined above and in the following,
with a compound of the general formulae (III) or (IIIa)

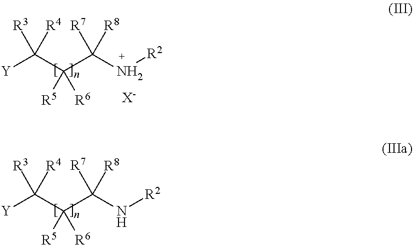

in which
n, R², R³, R⁴, R⁵, R⁶, R⁷ and R⁸ have one of the meanings defined above and in the following,
X⁻ is an anion equivalent, and
Y is a leaving group.

Also provided is a process for preparing compounds of the general formula (I-B.1) or (I-B.2)

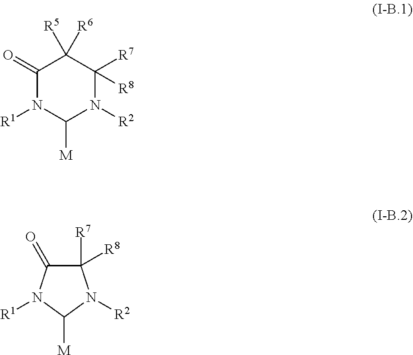

Where M, R¹, R², R⁵, R⁶, R⁷ and R⁸ have a meaning as defined above and in the following.

In one or more embodiments, this process comprises:

a1) the reaction of an isonitrile complex of the general formula (II)

R¹—N≡C-M (II)
in which R¹ and M have one of the meanings as defined above and in the following,
with a compound of the general formulae (III-B.1), (III-B.1.a), (III-B.2) or (III-B.2.a)

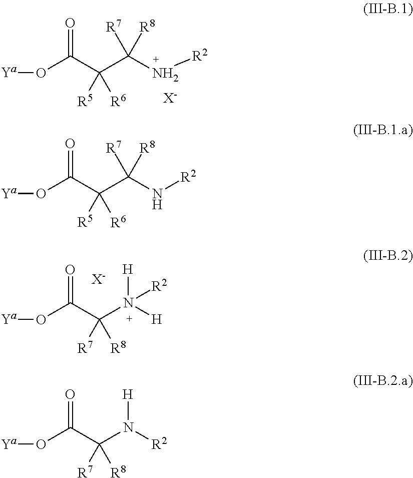

in which
R², R⁵, R⁶, R⁷ and R⁸ have one of the meanings as defined above and in the following,
X⁻ is an anion equivalent, and
Y^{a} is unsubstituted or substituted alkyl, unsubstituted or substituted aryl, unsubstituted or substituted arylcarbonyl or unsubstituted or substituted alkyl carbonyl.

Insofar the keto compounds of the general formula (I-B.1) or (I-B.2) are able to form tautomers, those tautomers are also part of the invention.

Embodiments of the invention also relate to a process for preparing compounds of the general formula (I-C.1) or (I-C.2)

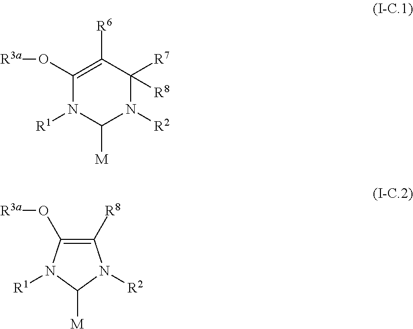

where
M, R¹, R², R^{3a}, R⁶, R⁷ and R⁸ have a meaning as defined above.

In one or more embodiments, this process comprises:
a1) the reaction of an isonitrile complex of the general formula (II)

R¹—N≡C-M (II)
in which R¹ and M have one of the meanings given above,
with a compound of the general formulae (III-C.1), (III-C.1.a), (III-C.2) or (III-C.2.a)

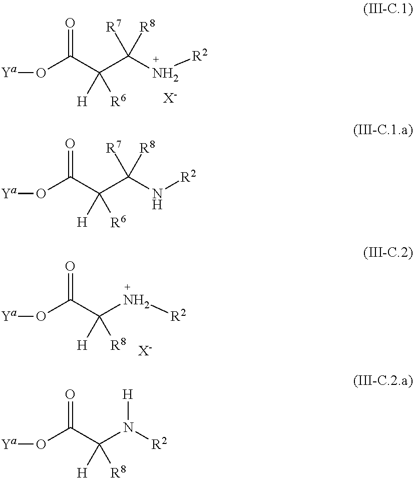

in which
R², R⁶, R⁷ and R⁸ have one of the meanings given above,
X⁻ is an anion equivalent, and
Y^{a} is unsubstituted or substituted alkyl, unsubstituted or substituted aryl, unsubstituted or substituted arylcarbonyl or unsubstituted or substituted alkyl carbonyl, and
b1) subjecting the product obtained in step a1) to a further reaction with a compound R^{3a}—Z, where Z is a leaving group, in the presence of a base.

In a second aspect, the invention provides a process for preparing compounds of the general formula (I-E) (compounds of the formula I, where n is 0 and R⁴ and R⁷ stand for the bond equivalent of a double bond between the carbon atoms carrying R⁴ and R⁷)

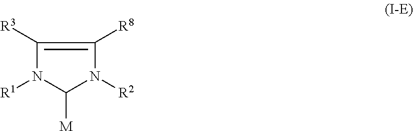

where
M is a metal atom containing group,
R¹ is selected from hydrogen, alkyl, cycloalkyl, heterocycloalkyl, aryl and hetaryl,
R² is selected from hydrogen, alkyl, cycloalkyl, heterocycloalkyl, aryl and hetaryl, wherein R¹ and R² do not both stand for hydrogen,
R³ is selected from hydrogen and in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
R⁸ is selected from hydrogen and in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
wherein the two radicals R² and R⁸ may also form together with the N atom to which R² is bound a 3- to 12-membered, unsubstituted or substituted nitrogen heterocycle which may optionally have 1, 2 or 3 further heteroatoms or heteroatom containing groups independently selected from O, N, NR^{a} and S as ring members, wherein R^{a} is hydrogen, alkyl, cycloalkyl or aryl.

Embodiments in accordance with this aspect provide a process comprising:

a2) the reaction of an isonitrile complex of the general formula II

R¹—N≡C-M (II)
where R¹ and M have one of the meanings given above,
with a compound of the general formula (V)

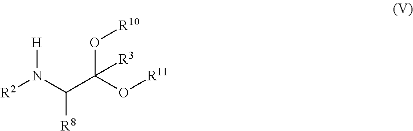

where
R², R³ and R⁸ have one of the meanings given above; and
R¹⁰ and R¹¹ are independently selected from C₁-C₄-alkyl or R¹⁰ and R¹¹ together are linear C₂-C₄-alkylene which may be substituted by one or more, e.g. 1, 2, 3, 4, 5 or 6 C₁-C₄ alkyl radicals;
to give an intermediate compound of the formula (VI)

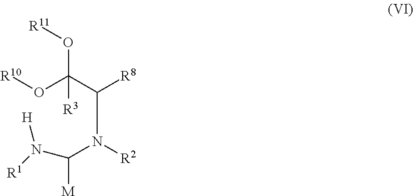

in which R¹, R², R³, R⁸, R¹⁰, R¹¹ and M are as defined above;
and
   b2) the treatment of the intermediate compound of the formula (VI) with an acid.

According to a certain embodiments, the process of the invention according to the second aspect is used for the formation of unsymmetrically substituted compounds of the formula (I-E). In some embodiments, R¹ and R² have different meanings.

In a third aspect, the invention provides a process for preparing compounds of the general formula I-F (compounds of the formula I, where n is 0 and R³ is CH₂-EWG)

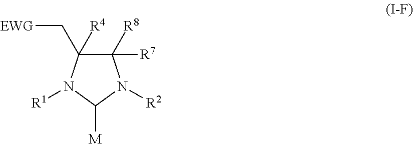

where R¹, R², R⁴, R⁷, R⁸, M and EWG have one of the meanings given above.

In accordance with this aspect, embodiments of the process comprise:

a3) the reaction of an isonitrile complex of the general formula II,

R¹—N≡C-M (II)
where R¹ and M have one of the meanings given above
with a compound of the general formulae (IIIb) or (IIIc)

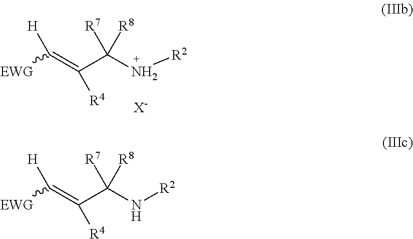

where
EWG, R², R⁴, R⁷ and R⁸ have one of the meanings given above; and
X⁻ is an anion equivalent.

According to certain embodiments, the process according to the third aspect is used for the formation of unsymmetrically substituted compounds of the formula (I-F). In some embodiments R¹ and R² have different meanings.

In a further aspect, embodiments of the invention provide new compounds of the general formula (I).

In a further aspect, embodiments of the invention provide new compounds of the general formula (VI).

In a further aspect, embodiments of the invention provide a catalyst, comprising or consisting of a compound of the general formula (I).

In a further aspect, provided is a method of forming a C—C, C—O, C—N or C—H bond comprising using the compound of the general formula (I) as or in a catalyst employed in a C—C, C—O, C—N or C—H bond formation reaction.

According to a certain embodiment, the compound of the general formula (I) is used in a C—C coupling reaction selected from the Suzuki reaction, Heck reaction, Sonogashira reaction, Stille reaction, Hartwig-Buchwald reaction and Kumada reaction.

According to another embodiment, the compound of the general formula (I) is used in a hydrogenation, hydroformylation, hydrosilylation, Hartwig-Buchwald reaction or amide α-arylation.

### DETAILED DESCRIPTION

It has now been found that, surprisingly, that reaction of substituted ω-haloalkylammonium salts or ω-(alkoxycarbonyl)alkylammonium salts with conveniently accessible isonitrile complexes can provide unsymmetrically disubstituted NHC-complexes.

As used herein, the term N-heterocyclic carbene (NHC) denotes compounds that can be present in the form of different resonance structures, represented e.g. by structures with a divalent carbon atom as well as ylide type structures.

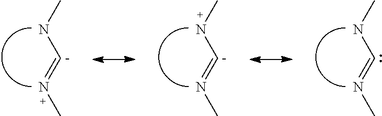

Preferred embodiments of the compounds of the general formula (I) are compounds of the formulae (I-A.1), (I-A.2), (I-B.1), (I-B.2), (I-C.1) and (I-C.2), as shown above and in the following. All definitions regarding compounds of the general formula (I) are also applicable for compounds of the formulae (I-A.1), (I-A.2), (I-B.1), (I-B.2), (I-C.1) and (I-C.2), unless explicitly defined otherwise. Preferred embodiments of the compounds of the general formula (I) are also compounds of the formulae (I-E) and (I-F), as shown above and in the following. All definitions regarding compounds of the general formula (I) are also applicable for compounds of the formulae (I-E) and (I-F), unless explicitly defined otherwise.

Single bonds are used to represent the M-C_{carbene} interactions in the compounds of the formulae (I) and (VI).

The expression “halogen” denotes in each case fluorine, bromine, chlorine or iodine, particularly chlorine, bromide or iodine.

As used herein, the expression “unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino” represents unsubstituted or substituted alkyl, unsubstituted or substituted alkoxy, unsubstituted or substituted alkylthio, unsubstituted or substituted (monoalkyl)amino, unsubstituted or substituted (dialkyl)amino, unsubstituted or substituted cycloalkyl, unsubstituted or substituted cycloalkoxy, unsubstituted or substituted cycloalkylthio, unsubstituted or substituted (monocycloalkyl)amino, unsubstituted or substituted (dicycloalkyl)amino, unsubstituted or substituted heterocycloalkyl, unsubstituted or substituted heterocycloalkoxy, unsubstituted or substituted heterocycloalkylthio, unsubstituted or substituted (monoheterocycloalkyl)amino, unsubstituted or substituted (diheterocycloalkyl)amino, unsubstituted or substituted aryl, unsubstituted or substituted aryloxy, unsubstituted or substituted arylthio, unsubstituted or substituted (monoaryl)amino, unsubstituted or substituted (diaryl)amino, unsubstituted or substituted hetaryl, unsubstituted or substituted hetaryloxy, unsubstituted or substituted hetarylthio, unsubstituted or substituted (monohetaryl)amino and unsubstituted or substituted (dihetaryl)amino.

As used herein, the expression “alkyl” comprises straight-chain or branched alkyl groups. Alkyl is preferably C₁-C₃₀-alkyl, more preferably C₁-C₂₀-alkyl and most preferably C₁-C₁₂-alkyl. Examples of alkyl groups are especially methyl, ethyl, n-propyl, iso-propyl, n-butyl, isobutyl, sec-butyl, tert-butyl, n-pentyl, neo-pentyl, n-hexyl, n-heptyl, n-octyl, n-nonyl, n-decyl, n-undecyl, n-dodecyl, n-tridecyl, n-tetradecyl, n-hexadecyl, n-octadecyl and n-eicosyl.

The expression alkyl also comprises alkyl radicals whose carbon chains may be interrupted by one or more nonadjacent groups which are selected from —O—, —S—, —NR^{b}—, —C(═O)—, —S(═O)— and/or —S(═O)₂—. R^{b} is preferably hydrogen, alkyl, cycloalkyl, heterocycloalkyl, aryl or hetaryl.

Substituted alkyl groups may, depending on the length of the alkyl chain, have one or more (e.g. 1, 2, 3, 4, 5 or more than 5) substituents. These are preferably each independently selected from cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE¹E² where E¹ and E² are each independently hydrogen, alkyl, cycloalkyl, heterocycloalkyl, aryl or hetaryl. Cycloalkyl, heterocycloalkyl, aryl and hetaryl substituents of the alkyl groups may in turn be unsubstituted or substituted; suitable substituents are the substituents mentioned below for these groups.

Carboxylate and sulfonate respectively represent a derivative of a carboxylic acid function and a sulfonic acid function, especially a metal carboxylate or sulfonate, a carboxylic ester or sulfonic ester function or a carboxamide or sulfonamide function.

The above remarks regarding alkyl also apply to the alkyl moiety in alkoxy, alkylthio (=alkylsulfanyl), monoalkylamino and dialkylamino.

As used herein, the term “cycloalkyl” denotes a mono-, bi- or tricyclic hydrocarbon radical having usually from 3 to 20, preferably 3 to 12, more preferably 5 to 12, carbon atoms such as cyclopropyl, cyclobutyl, cyclopentyl, cyclohexyl, cycloheptyl, cyclooctyl, cyclododecyl, cyclopentadecyl, norbornyl, bicyclo[2.2.2]octyl or adamantyl.

Substituted cycloalkyl groups may, depending on the ring size, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE³E⁴ where E³ and E⁴ are each independently hydrogen, alkyl, cycloalkyl, heterocycloalkyl, aryl or hetaryl. In the case of substitution, the cycloalkyl groups preferably bear one or more, for example one, two, three, four or five, C₁-C₆-alkyl groups. Examples of substituted cycloalkyl groups are especially 2- and 3-methylcyclopentyl, 2- and 3-ethylcyclopentyl, 2-, 3- and 4-methylcyclohexyl, 2-, 3- and 4-ethylcyclohexyl, 2-, 3- and 4-propylcyclohexyl, 2-, 3- and 4-isopropylcyclohexyl, 2-, 3- and 4-butylcyclohexyl, 2-, 3- and 4-sec.-butylcyclohexyl, 2-, 3- and 4-tert-butylcyclohexyl, 2-, 3- and 4-methylcycloheptyl, 2-, 3- and 4-ethylcycloheptyl, 2-, 3- and 4-propyl-cycloheptyl, 2-, 3- and 4-isopropylcycloheptyl, 2-, 3- and 4-butylcycloheptyl, 2-, 3- and 4-sec-butylcycloheptyl, 2-, 3- and 4-tert-butylcycloheptyl, 2-, 3-, 4- and 5-methyl-cyclooctyl, 2-, 3-, 4- and 5-ethylcyclooctyl, 2-, 3-, 4- and 5-propylcyclooctyl.

The above remarks regarding cycloalkyl also apply to the cycloalkyl moiety in cycloalkoxy, cycloalkylthio (=cycloalkylsulfanyl), monocycloalkylamino and dicycloalkylamino.

As used herein, the term “aryl” refers to mono- or polycyclic aromatic hydrocarbon radicals. Aryl usually is an aromatic radical having 6 to 24 carbon atoms, preferably 6 to 20 carbon atoms, especially 6 to 14 carbon atoms as ring members. Aryl is preferably phenyl, naphthyl, indenyl, fluorenyl, anthracenyl, phenanthrenyl, naphthacenyl, chrysenyl, pyrenyl, coronenyl, perylenyl, etc., and more preferably phenyl or naphthyl.

Substituted aryls may, depending on the number and size of their ring systems, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE⁵E⁶ where E⁵ and E⁶ are each independently hydrogen, alkyl, cycloalkyl, heterocycloalkyl, aryl or hetaryl. The alkyl, alkoxy, alkylamino, alkylthio, cycloalkyl, heterocycloalkyl, aryl and hetaryl substituents on the aryl may in turn be unsubstituted or substituted. Reference is made to the substituents mentioned above for these groups. The substituents on the aryl are preferably selected from alkyl, alkoxy, haloalkyl, haloalkoxy, aryl, fluorine, chlorine, bromine, cyano and nitro. Substituted aryl is more preferably substituted phenyl which generally bears 1, 2, 3, 4 or 5, preferably 1, 2 or 3, substituents.

Substituted aryl is preferably aryl substituted by at least one alkyl group (“alkaryl”, also referred to hereinafter as alkylaryl). Alkaryl groups may, depending on the size of the aromatic ring system, have one or more (e.g. 1, 2, 3, 4, 5, 6, 7, 8, 9 or more than 9) alkyl substituents. The alkyl substituents may be unsubstituted or substituted. In this regard, reference is made to the above statements regarding unsubstituted and substituted alkyl. In a preferred embodiment, the alkaryl groups have exclusively unsubstituted alkyl substituents. Alkaryl is preferably phenyl which bears 1, 2, 3, 4 or 5, preferably 1, 2 or 3, more preferably 1 or 2, alkyl substituents.

Aryl which bears one or more radicals is, for example, 2-, 3- and 4-methylphenyl, 2,4-, 2,5-, 3,5- and 2,6-dimethylphenyl, 2,4,6-trimethylphenyl, 2-, 3- and 4-ethylphenyl, 2,4-, 2,5-, 3,5- and 2,6-diethylphenyl, 2,4,6-triethylphenyl, 2-, 3- and 4-propylphenyl, 2,4-, 2,5-, 3,5- and 2,6-dipropylphenyl, 2,4,6-tripropylphenyl, 2-, 3- and 4-isopropylphenyl, 2,4-, 2,5-, 3,5- and 2,6-diisopropylphenyl, 2,4,6-triisopropylphenyl, 2-, 3- and 4-butylphenyl, 2,4-, 2,5-, 3,5- and 2,6-dibutylphenyl, 2,4,6-tributylphenyl, 2-, 3- and 4-isobutylphenyl, 2,4-, 2,5-, 3,5- and 2,6-diisobutylphenyl, 2,4,6-triisobutylphenyl, 2-, 3- and 4-sec-butylphenyl, 2,4-, 2,5-, 3,5- and 2,6-di-sec-butylphenyl, 2,4,6-tri-sec-butylphenyl, 2-, 3- and 4-tert-butylphenyl, 2,4-, 2,5-, 3,5- and 2,6-di-tert-butylphenyl and 2,4,6-tri-tert-butylphenyl; 2-, 3- and 4-methoxyphenyl, 2,4-, 2,5-, 3,5- and 2,6-dimethoxyphenyl, 2,4,6-trimethoxyphenyl, 2-, 3- and 4-ethoxyphenyl, 2,4-, 2,5-, 3,5- and 2,6-diethoxyphenyl, 2,4,6-triethoxyphenyl, 2-, 3- and 4-propoxyphenyl, 2,4-, 2,5-, 3,5- and 2,6-dipropoxyphenyl, 2-, 3- and 4-isopropoxyphenyl, 2,4-, 2,5-, 3,5- and 2,6-diisopropoxyphenyl and 2-, 3- and 4-butoxyphenyl; 2-, 3- and 4-chlorophenyl, (2-chloro-6-methyl)phenyl, (2-chloro-6-ethyl)phenyl, (4-chloro-6-methyl)phenyl, (4-chloro-6-ethyl)phenyl.

The above remarks regarding aryl also apply to the aryl moiety in aryloxy, arylthio (=arylsulfanyl), monoarylamino and diarylamino.

As used herein, the expression “heterocycloalkyl” comprises nonaromatic, unsaturated or fully saturated, cycloaliphatic groups having generally 5 to 8 ring atoms, preferably 5 or 6 ring atoms. In the heterocycloalkyl groups, compared to the corresponding cycloalkyl groups, 1, 2, 3, 4 or more than 4 of the ring carbon atoms are replaced by heteroatoms or heteroatom-containing groups. The heteroatoms or heteroatom-containing groups are preferably selected from —O—, —S—, —NR^{e}—, —C(═O)—, —S(═O)— and/or —S(═O)₂—. R^{e} is preferably hydrogen, alkyl, cycloalkyl, heterocycloalkyl, aryl or hetaryl. Heterocycloalkyl is unsubstituted or optionally bears one or more, e.g. 1, 2, 3, 4, 5, 6 or 7, identical or different radicals. These are preferably each independently selected from alkyl, alkoxy, alkylamino, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE⁵E⁶ where E⁵ and E⁶ are each independently hydrogen, alkyl, cycloalkyl, heterocycloalkyl, aryl or hetaryl. Examples of heterocycloalkyl groups are especially pyrrolidinyl, piperidinyl, 2,2,6,6-tetramethylpiperidinyl, imidazolidinyl, pyrazolidinyl, oxazolidinyl, morpholinyl, thiazolidinyl, isothiazolidinyl, isoxazolidinyl, piperazinyl, tetrahydrothiophenyl, dihydrothien-2-yl, tetrahydrofuranyl, dihydrofuran-2-yl, tetrahydropyranyl, 2-oxazolinyl, 3-oxazolinyl, 4-oxazolinyl and dioxanyl.

Substituted heterocycloalkyl groups may, depending on the ring size, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE⁷E⁸ where E⁷ and E⁸ are each independently hydrogen, alkyl, cycloalkyl, heterocycloalkyl, aryl or hetaryl. In the case of substitution, the heterocycloalkyl groups preferably bear one or more, for example one, two, three, four or five, C₁-C₆-alkyl groups.

The above remarks regarding heterocycloalkyl also apply to the heterocycloalkyl moiety in heterocycloalkoxy, heterocycloalkylthio (=heterocycloalkylsulfanyl), monoheterocycloalkylamino and diheterocycloalkylamino.

As used herein, the expression “hetaryl” (heteroaryl) comprises heteroaromatic, mono- or polycyclic groups. In addition to the ring carbon atoms, these have 1, 2, 3, 4 or more than 4 heteroatoms as ring members. The heteroatoms are preferably selected from oxygen, nitrogen, selenium and sulfur. The hetaryl groups have preferably 5 to 18, e.g. 5, 6, 8, 9, 10, 11, 12, 13 or 14, ring atoms.

Monocyclic hetaryl groups are preferably 5- or 6-membered hetaryl groups, such as 2-furyl (furan-2-yl), 3-furyl (furan-3-yl), 2-thienyl (thiophen-2-yl), 3-thienyl (thiophen-3-yl), selenophen-2-yl, selenophen-3-yl, 1H-pyrrol-2-yl, 1H-pyrrol-3-yl, pyrrol-1-yl, imidazol-2-yl, imidazol-1-yl, imidazol-4-yl, pyrazol-1-yl, pyrazol-3-yl, pyrazol-4-yl, pyrazol-5-yl, 3-isoxazolyl, 4-isoxazolyl, 5-isoxazolyl, 3-isothiazolyl, 4-isothiazolyl, 5-isothiazolyl, 2-oxazolyl, 4-oxazolyl, 5-oxazolyl, 2-thiazolyl, 4-thiazolyl, 5-thiazolyl, 1,2,4-oxadiazol-3-yl, 1,2,4-oxadiazol-5-yl, 1,3,4-oxadiazol-2-yl, 1,2,4-thiadiazol-3-yl, 1,2,4-thiadiazol-5-yl, 1,3,4-thiadiazol-2-yl, 4H-[1,2,4]-triazol-3-yl, 1,3,4-triazol-2-yl, 1,2,3-triazol-1-yl, 1,2,4-triazol-1-yl, pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Polycyclic hetaryl groups have 2, 3, 4 or more than 4 fused rings. The fused-on rings may be aromatic, saturated or partly unsaturated. Examples of polycyclic hetaryl groups are quinolinyl, isoquinolinyl, indolyl, isoindolyl, indolizinyl, benzofuranyl, isobenzofuranyl, benzothiophenyl, benzoxazolyl, benzisoxazolyl, benzthiazolyl, benzoxadiazolyl, benzothiadiazolyl, benzoxazinyl, benzopyrazolyl, benzimidazolyl, benzotriazolyl, benzotriazinyl, benzoselenophenyl, thienothiophenyl, thienopyrimidyl, thiazolothiazolyl, dibenzopyrrolyl (carbazolyl), dibenzofuranyl, dibenzothiophenyl, naphtho[2,3-b]thiophenyl, naphtha-[2,3-b]furyl, dihydroindolyl, dihydroindolizinyl, dihydroisoindolyl, dihydroquinolinyl and dihydroisoquinolinyl.

Substituted hetaryl groups may, depending on the number and size of their ring systems, have one or more (e.g. 1, 2, 3, 4, 5 or more than 5) substituents. These are preferably each independently selected from alkyl, alkoxy, alkylthio, cycloalkyl, heterocycloalkyl, aryl, hetaryl, fluorine, chlorine, bromine, hydroxyl, mercapto, cyano, nitro, nitroso, formyl, acyl, COOH, carboxylate, alkylcarbonyloxy, carbamoyl, SO₃H, sulfonate, sulfamino, sulfamide, amidino, NE⁹E¹⁰ where E⁹ and E¹⁰ are each independently hydrogen, alkyl, cycloalkyl, heterocycloalkyl, aryl or hetaryl. Halogen substituents are preferably fluorine, chlorine or bromine. The substituents are preferably selected from C₁-C₆-alkyl, C₁-C₆-alkoxy, hydroxyl, carboxyl, halogen and cyano.

The above remarks regarding hetaryl also apply to the hetaryl moiety in hetaryloxy, hetarylthio, monohetarylamino and dihetarylamino.

Preferably, in the compounds of the general formula (I) the metal atom containing group M comprises a metal selected from groups 4, 5, 6, 7, 8, 9, 10, 11, 12 and 13 of the Periodic Table. Preferred metals are Ti, Zr, Cr, Mo, W, Re, Fe, Ru, Co, Rh, Ir, Ni, Pd, Pt, Cu, Ag, Au, Zn, In and B.

Particular preference is given to compounds of the general formula (I), wherein M is a Pd(II), Pt(II) or Au(I) containing group.

Suitable groups M are in principle groups of the formula L_{y}Me-, wherein Me is a metal, L is a ligand and y is an integer which depends on the valence and type of the metal and on the number of coordination sites occupied by each of the ligands L. Suitable ligands L are independently selected from among F, Cl, Br, I, CO, isonitriles, nitriles, amines, carboxylates, acetylacetonate, hydride, olefins, cycloolefins, dienes, alkynes, C₅H₅⁻, C₇H₇⁺, N-containing heterocycles, aromatics, heteroaromatics, ethers, PF₃, phospholes, phosphabenzenes, phosphines (e.g. P(C₆H₅)₃, P(CH₃)(C₆H₅)₂, P(CH₃)₂(C₆H₅), P(CH₃)₃, dppe (1,2-ethanediylbis[(diphenyl)phosphine]), P(C₆H₁₁)₃, etc.), phosphinites, phosphonites, phosphites, alkylsulfonates, arylsulfonates, etc.

In particular, in the compounds of the general formula (I), M is selected from PdCl₂(CNR¹), PtCl₂(CNR¹), PdCl(CNR¹)₂, Au (CNR¹) and AuCl, where R¹ is selected from hydrogen, alkyl, cycloalkyl, heterocycloalkyl, aryl and hetaryl.

Preferably, R¹ is selected from groups of the formulae IV.1 to IV.5, with particular preference given to the formulae IV.1 and IV.2:

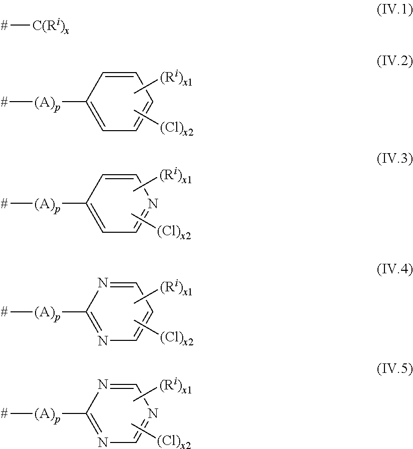

in which
# represents the bonding site to the nitrogen atom,
p is 0 or 1,
   x is 2 or 3, where, in the case that x is 2, the carbon atom which bears the Rⁱ radicals additionally bears 1 hydrogen atom,
x₁ in the formulae IV.2, IV.3 and IV.4 is 0, 1, 2, 3, 4, or 5,
x₂ in the formulae IV.2, IV.3 and IV.4 is 0 or 1,
   with the proviso that the sum of x₁ and x₂ in the formulae IV.2, IV.3 and IV.4 is 0, 1, 2, 3, 4 or 5,
x₁ in the formula IV.5 is 0, 1 or 2,
x₂ in the formula IV.5 is 0 or 1,
   with the proviso that the sum of x₁ and x₂ in the formulae IV.5 is 0, 1 or 2,
A where present, is a C₁-C₁₀-alkylene group which may be interrupted by one or more nonadjacent groups which are selected from —O— and —S—,
the R¹ radicals are each independently selected from C₁-C₃₀-alkyl, C₁-C₃₀-alkyloxy or C₁-C₃₀-alkylthio, wherein the alkyl chain in alkyl, alkyloxy or alkylthio may be interrupted by one or more nonadjacent oxygen atom(s).

More preferably, in the formulae IV.2, IV.3 and IV.4, x₁ is 0, 1, 2 or 3, x₂ is 0 or 1, with the proviso that the sum of x₁ and x₂ is 0, 1, 2 or 3.

More preferably, R¹ is selected from C₁-C₆-alkyl, phenyl and phenyl which carries 1, 2 or 3 radicals independently selected from C₁-C₆-alkyl and chlorine. Especially, R¹ is selected from isopropyl, tert.-butyl, 2-methylphenyl, 3-methylphenyl, 4-methylphenyl, 2,4-dimethylphenyl, 2,5-dimethylphenyl, 3,5-dimethylphenyl, 2,6-dimethylphenyl, 2,4,6-trimethylphenyl, 2-ethylphenyl, 3-ethylphenyl, 4-ethylphenyl, 2,4-diethylphenyl, 2,5-diethylphenyl, 3,5-diethylphenyl, 2,6-diethylphenyl, 2,4,6-triethylphenyl, 2-propylphenyl, 3-propylphenyl, 4-propylphenyl, 2,4-dipropylphenyl, 2,5-dipropylphenyl, 3,5-dipropylphenyl, 2,6-dipropylphenyl, 2,4,6-tripropylphenyl, 2-isopropylphenyl, 3-isopropylphenyl, 4-isopropylphenyl, 2,4-diisopropylphenyl 2,5-diisopropylphenyl, 3,5-diisopropylphenyl, 2,6-diisopropylphenyl, 2,4,6-triisopropylphenyl, 2-butylphenyl, 3-butylphenyl, 4-butylphenyl, 2,4-dibutylphenyl, 2,5-dibutylphenyl, 3,5-dibutylphenyl, 2,6-dibutylphenyl, 2,4,6-tributylphenyl, 2-isobutylphenyl, 3-isobutylphenyl, 4-isobutylphenyl, 2,4-diisobutylphenyl, 2,5-diisobutylphenyl, 3,5-diisobutylphenyl, 2,6-diisobutylphenyl, 2,4,6-triisobutylphenyl, 2-sec-butylphenyl, 3-sec-butylphenyl, 4-sec-butylphenyl, 2,4-di-sec-butylphenyl, 2,5-di-sec-butylphenyl, 3,5-di-sec-butylphenyl, 2,6-di-sec-butylphenyl, 2,4,6-tri-sec-butylphenyl, 2-tert-butylphenyl, 3-tert-butylphenyl, 4-tert-butyl-phenyl, 2,4-di-tert-butylphenyl, 2,5-di-tert-butylphenyl, 3,5-di-tert-butylphenyl, 2,6-di-tert-butylphenyl, 2,4,6-tri-tert-butylphenyl, 2-chlorophenyl, 3-chlorophenyl, 4-chlorophenyl, (2-chloro-6-methyl)phenyl, (2-chloro-6-ethyl)phenyl, (2-chloro-6-propyl)phenyl, (2-chloro-6-isopropyl)phenyl, (2-chloro-6-butyl)phenyl, (2-chloro-6-isobutyl)phenyl, (2-chloro-6-sec-butyl)phenyl and (2-chloro-6-tert-butyl)phenyl.

In particular, R¹ is selected from tert.-butyl, 2,6-dimethylphenyl, 2,4,6-trimethylphenyl, 2,6-diisopropylphenyl and (2-chloro-6-methyl)phenyl.

Preferably, R² is selected from hydrogen, alkyl and cycloalkyl, in particular C₁-C₆-alkyl, phenyl-C₁-C₆-alkyl and C₅-C₁₅-cycloalkyl.

More preferably R² is selected from methyl, ethyl, n-propyl, isopropyl, n-butyl, sec-butyl, tert-butyl, 1-phenylethyl, cyclopentyl, cyclohexyl, cyclododecyl, cyclopentadecyl and 1-adamantyl.

According to one or more embodiments, R³ and R⁴ are independently selected from hydrogen and unsubstituted or substituted aryl. If at least one of the residues R³ and R⁴ is aryl then this residue is preferably phenyl. In particular, R³ and R⁴ are both hydrogen.

According to one or more embodiments, R³ and R⁴ together with the carbon atom to which they are bound are C═O.

According to one or more embodiments, R³ is a group O—R^{3a}, where R^{3a} is a group bound to the oxygen via a carbon atom, silicon atom, sulphur atom, phosphorus, boron or titanium atom. In this embodiment, for compounds of the general formula (I), wherein n is 0, the residues R⁴ and R⁷ stand for the bond equivalent of a double bond between the carbon atoms carrying R⁴ and R⁷. In this embodiment, for compounds of the general formula (I), wherein n is 1, the residues R⁴ and R⁵ stand for the bond equivalent of a double bond between the carbon atoms carrying R⁴ and R⁵.

Preferably, R^{3a} is selected from groups of the formulae V-A, V-B, V-C, V-D, V-E, V-F, V-G, V-H, V-I, V-K or V-L, with more preference given to the groups of the formulae V-A, V-B or V-C,

wherein

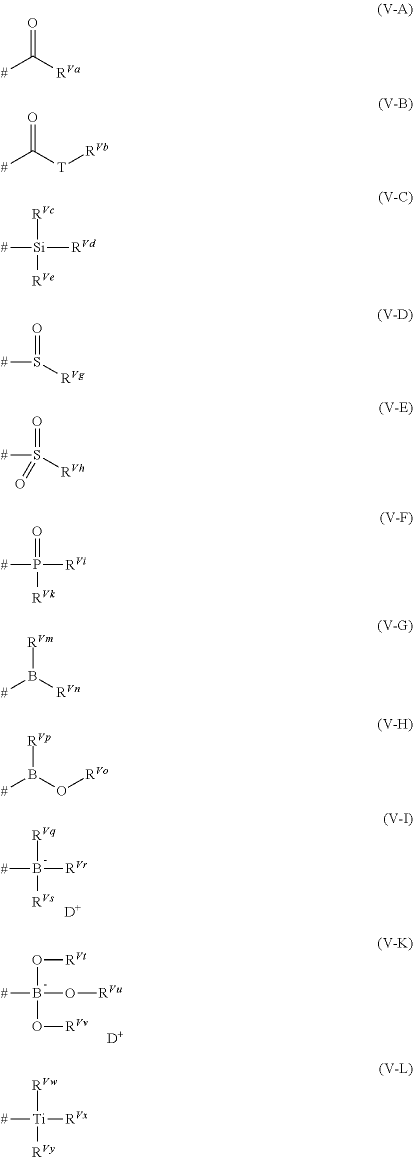

# represents the bonding site to the oxygen atom,
T is selected from —O— and —NR^{Vf}, wherein R^{Vf} is hydrogen, alkyl, cycloalkyl or aryl,
R^{Va}, R^{Vb} are selected from unsubstituted or substituted alkyl, unsubstituted or substituted cycloalkyl, unsubstituted or substituted aryl and unsubstituted or substituted hetaryl,
R^{Vc}, R^{Vd}, R^{Ve} are independently of each other selected from unsubstituted or substituted alkyl, unsubstituted or substituted cycloalkyl, unsubstituted or substituted aryl and unsubstituted or substituted hetaryl,
R^{Vg} is selected from unsubstituted or substituted heterocycloalkyl,
R^{Vh} is selected from unsubstituted or substituted alkyl, unsubstituted or substituted alkenyl, unsubstituted or substituted cycloalkyl, unsubstituted or substituted aryl and unsubstituted or substituted hetaryl,
R^{Vi} and R^{Vk} are independently of each other selected from unsubstituted or substituted alkyl, unsubstituted or substituted cycloalkyl, unsubstituted or substituted aryl, alkoxy, unsubstituted or substituted aryloxy and unsubstituted or substituted cycloalkyloxy,
R^{Vm} and R^{Vn}, are independently of each other selected from unsubstituted or substituted alkyl, unsubstituted or substituted alkenyl, unsubstituted or substituted cycloalkyl, unsubstituted or substituted aryl and unsubstituted or substituted hetaryl,
R^{Vo} and R^{Vp}, are independently of each other selected from unsubstituted or substituted alkyl, unsubstituted or substituted alkenyl, unsubstituted or substituted cycloalkyl, unsubstituted or substituted aryl and unsubstituted or substituted hetaryl,
R^{Vq}, R^{Vr} and R^{Vs}, are independently of each other selected from unsubstituted or substituted alkyl, unsubstituted or substituted alkenyl, unsubstituted or substituted cycloalkyl, unsubstituted or substituted aryl and unsubstituted or substituted hetaryl,
R^{Vt}, R^{Vu} and R^{Vv}, are independently of each other selected from unsubstituted or substituted alkyl, unsubstituted or substituted alkenyl, unsubstituted or substituted cycloalkyl, unsubstituted or substituted aryl and unsubstituted or substituted hetaryl, and
R^{Vw}, R^{Vx} and R^{Vy} are independently of each other selected from unsubstituted or substituted alkyloxy, unsubstituted or substituted alkenyloxy, unsubstituted or substituted cycloalkyloxy and unsubstituted or substituted aryloxy; and
D⁺ is a cation equivalent.

The cation equivalent D⁺ serves merely as counterion and can be selected freely from among monovalent cations and the parts of polyvalent cations corresponding to a single positive charge. Suitable cations are, for example, alkali metal ions D⁺, e.g. Na⁺ and K⁺, earth alkali metal cations, e.g. Ca²⁺, ammonium ions.

Preferably, R^{Va} is phenyl, which is unsubstituted or substituted by 1, 2 or 3 radicals independently selected from C₁-C₄-alkyl, C₁-C₄-alkoxy and nitro.

Preferably, R^{Vb} is (C₁-C₄-alkyl)phenyl, wherein the phenyl moiety of (C₁-C₄-alkyl)phenyl is unsubstituted or substituted by 1, 2 or 3 radicals independently selected from C₁-C₄-alkyl, C₁-C₄-alkoxy and nitro.

Preferably T is —O—. In the radical of the formula V-B, T is preferably —O— and R^{Vb} is benzyl wherein the phenyl moiety of benzyl is unsubstituted or substituted by 1, 2 or 3 radicals independently selected from C₁-C₄-alkyl, C₁-C₄-alkoxy and nitro.

Preferably, R^{Vc}, R^{Vd} and R^{Ve} are independently of each other selected from C₁-C₆-alkyl, C₅-C₁₀-cycloalkyl and phenyl which is unsubstituted or substituted by 1, 2 or 3 radicals independently selected from C₁-C₄-alkyl and C₁-C₄-alkoxy. Examples for a radical V-C are trimethylsilyl, triethylsilyl, triisopropylsilyl, dimethylisopropylsilyl, diethylisopropylsilyl, dimethylhexylsilyl, 2-norbornyldimethylsilyl, tert-butyldimethylsilyl, di-tert-butylmethylsilyl, tert-butyldiphenylsilyl, tribenzylsilyl, triphenylsilyl and diphenylmethylsilyl.

Preferably, the radical of the formula V-D is 2,2,5,5-tetramethylpyrrolidin-3-one-1-sulfinate.

Preferably, R^{Vb} is C₁-C₄-alkyl, C₁-C₄-haloalkyl, C₁-C₄-alkyl substituted by phenyl wherein phenyl is unsubstituted or substituted by 1, 2 or 3 radicals independently selected from C₁-C₄-alkyl, C₁-C₄-alkoxy, C₁-C₄-haloalkyl, C₁-C₄-haloalkoxy and nitro, C₂-C₆-alkenyl, benzyl, phenyl, wherein phenyl is unsubstituted or substituted by 1, 2 or 3 radicals independently selected from C₁-C₄-alkyl, C₁-C₄-haloalkyl, C₁-C₄-haloalkoxy, C₁-C₄-alkoxy and nitro. Examples of radicals of the formula V-E are methanesulfonate, trifluoromethanesulfonate, 2-[(4-nitrophenyl)-ethyl]sulfonate, allylsulfonate, benzylsulfonate, tosylate, and 2-trifluoromethylbenzenesulfonate.

Preferably, R^{Vi} and R^{Vk} are independently of each other selected from C₁-C₆-alkyl, C₁-C₆-alkoxy and phenyl, which is unsubstituted or substituted by 1, 2 or 3 radicals independently selected from C₁-C₄-alkyl, C₁-C₄-haloalkyl, C₁-C₄-haloalkoxy and C₁-C₄-alkoxy.

Preferably, R⁷, R⁸, and, if present, R⁵ and R⁶ are independently selected from hydrogen and unsubstituted or substituted aryl. If at least one of the residues R⁷, R⁸, and, if present, R⁵ and R⁶ is aryl then this residue is preferably phenyl. In a certain embodiment, R⁷, R⁸, and, if present, R⁵ and R⁶ are all hydrogen. In another embodiment, one of the residues, selected from R⁷, R⁸, and, if present, R⁵ and R⁶ is phenyl and the other residues are all hydrogen.

In a certain embodiment, the two radicals R² and R⁸ may also form together with the N atom to which R² and the carbon atom to which R⁸ are bound a 3-, 4-, 5-, 6-, 7-, 8-, 9-, 10-, 11- or 12-membered, unsubstituted or substituted nitrogen heterocycle which may optionally have 1, 2 or 3 further heteroatoms or heteroatom containing groups independently selected from O, N, NR^{a} and S as ring members, wherein R^{a} is hydrogen, alkyl, cycloalkyl or aryl. Suitable substituents on the 3- to 12-membered nitrogen heterocycle are preferably halogen, cyano, nitro, hydroxy, mercapto, amino, carboxyl, C₁-C₆-alkyl, C₁-C₆-alkoxy, C₂-C₆-alkenyloxy, C₂-C₆-alkynyloxy, C₁-C₆-haloalkoxy, and C₁-C₆-alkylthio and/or two substituents that are bound to adjacent atoms of the 3- to 12-membered heterocycle form together with said atoms a fused benzene ring, a fused naphthalene ring, a fused saturated or partially unsaturated 5-, 6-, or 7-membered carbocycle or a fused 5-, 6-, or 7-membered heterocycle, which contains 1, 2, 3 or 4 heteroatoms selected from oxygen, sulfur and NR^{a}, wherein R^{a} is as defined above, as ring members, and wherein the fused ring is unsubstituted or may carry any combination of 1, 2, 3, or 4 radicals selected from halogen, cyano, nitro, hydroxy, mercapto, amino, carboxyl, C₁-C₆-alkyl, C₁-C₆-alkoxy, C₂-C₆-alkenyloxy, C₂-C₆-alkynyloxy, C₁-C₆-haloalkoxy, and C₁-C₆-alkylthio.

Preferred are compounds of the general formula (I-D)

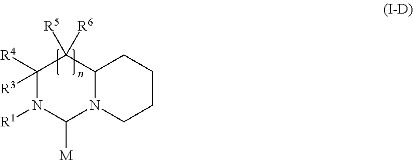

where n, M, R¹, R³, R⁴, R⁵ and R⁶ have the aforementioned meanings, in particular the meanings mentioned as preferred. In particular, n is 0, M is selected from PdCl₂(CNR¹), PdCl(CNR¹)₂, PtCl₂(CNR¹), Au(CNR¹) and AuC¹, R¹ is selected from tert.-butyl, 2,6-dimethylphenyl, 2,4,6-trimethylphenyl, 2,6-diisopropylphenyl and (2-chloro-6-methyl)phenyl, and R³ and R⁴ are both hydrogen.

In the aforementioned first variant, the process of the invention is employed for preparing compounds of the formula (I-A.1) or (I-A.2)

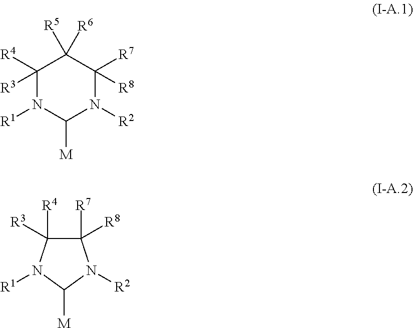

where
M, R¹, R², R⁵, R⁶, R⁷ and R⁸ have the aforementioned meanings, in particular the meanings mentioned as preferred,
R³ and R⁴ are independently selected from hydrogen and in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino.

In a preferred embodiment of the first variant, the process of the invention is employed for preparing compounds of the general formula (I-A.2.1)

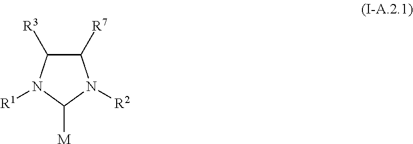

Where M, R¹, R², R³ and R⁷ have the aforementioned meanings, in particular the meanings mentioned as preferred.

(In the formula I-a.2.1 the residues R⁴ and R⁸ are hydrogen and are not depicted.)

The compounds of the general formula (I-A.2.2) which are indicated in Tables 1 to 5 below represent certain embodiments of the present invention. The meanings for R¹, R² and R³ indicated in Table A below represent embodiments of the invention which are likewise preferred independently of one another and especially in combination.

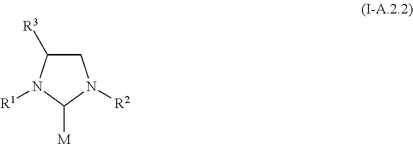

### Table 1

Compounds of the formula (I-A.2.2) in which the group M is PdCl₂(CNR¹) and the combination of R¹, R² and R³ for a compound in each case corresponds to one line of Table A. The residue R¹ bound to the ring nitrogen atom and the residue R¹ in the group PdCl₂(CNR¹) have both the same meaning. In comparison to the general formula (I), in formula (I-A.2.2) n is 0 and the residues R⁴, R⁷ and R⁸ are hydrogen and are not depicted.

### Table 2

Compounds of the formula (I-A.2.2) in which the group M is PtCl₂(CNR¹) and the combination of R¹, R² and R³ for a compound in each case corresponds to one line of Table A. The residue R¹ bound to the ring nitrogen atom and the residue R¹ in the group PtCl₂(CNR¹) have both the same meaning.

### Table 3

Compounds of the formula (I-A.2.2) in which the group M is PdCl(CNR¹)₂ and the combination of R¹, R² and R³ for a compound in each case corresponds to one line of Table A. The residue R¹ bound to the ring nitrogen atom and the residue R¹ in the group PdCl(CNR¹)₂ have both the same meaning.

### Table 4

Compounds of the formula (I-A.2.2) in which the group M is AuCl and the combination of R¹, R² and R³ for a compound in each case corresponds to one line of Table A.

### Table 5

Compounds of the formula (I-A.2.2) in which the group M is Au(CNR¹) and the combination of R¹, R² and R³ for a compound in each case corresponds to one line of Table A. The residue R¹ bound to the ring nitrogen atom and the residue R¹ in the group AuCl(CNR¹) have both the same meaning.

TABLE A

| No. | R¹ | R² | R³ |
|---|---|---|---|
| A-1 | 2,6-dimethylphenyl | methyl | H |
| A-2 | 2,6-dimethylphenyl | ethyl | H |
| A-3 | 2,6-dimethylphenyl | n-propyl | H |
| A-4 | 2,6-dimethylphenyl | isopropyl | H |
| A-5 | 2,6-dimethylphenyl | butyl | H |
| A-6 | 2,6-dimethylphenyl | isobutyl | H |
| A-7 | 2,6-dimethylphenyl | sec-buty | H |
| A-8 | 2,6-dimethylphenyl | tert-butyl | H |
| A-9 | 2,6-dimethylphenyl | cyclohexyl | H |
| A-10 | 2,6-dimethylphenyl | 1-adamantyl | H |
| A-11 | 2,6-dimethylphenyl | methyl | phenyl |
| A-12 | 2,6-dimethylphenyl | ethyl | phenyl |
| A-13 | 2,6-dimethylphenyl | n-propyl | phenyl |
| A-14 | 2,6-dimethylphenyl | isopropyl | phenyl |
| A-15 | 2,6-dimethylphenyl | butyl | phenyl |
| A-16 | 2,6-dimethylphenyl | isobutyl | phenyl |
| A-17 | 2,6-dimethylphenyl | sec-buty | phenyl |
| A-18 | 2,6-dimethylphenyl | tert-butyl | phenyl |
| A-19 | 2,6-dimethylphenyl | cyclohexyl | phenyl |
| A-20 | 2,6-dimethylphenyl | 1-adamantyl | phenyl |
| A-21 | 2,4,6-trimethylphenyl | methyl | H |
| A-22 | 2,4,6-trimethylphenyl | ethyl | H |
| A-23 | 2,4,6-trimethylphenyl | n-propyl | H |
| A-24 | 2,4,6-trimethylphenyl | isopropyl | H |
| A-25 | 2,4,6-trimethylphenyl | butyl | H |
| A-26 | 2,4,6-trimethylphenyl | isobutyl | H |
| A-27 | 2,4,6-trimethylphenyl | sec-buty | H |
| A-28 | 2,4,6-trimethylphenyl | tert-butyl | H |
| A-29 | 2,4,6-trimethylphenyl | cyclohexyl | H |
| A-30 | 2,4,6-trimethylphenyl | 1-adamantyl | H |
| A-31 | 2,4,6-trimethylphenyl | methyl | phenyl |
| A-32 | 2,4,6-trimethylphenyl | ethyl | phenyl |
| A-33 | 2,4,6-trimethylphenyl | n-propyl | phenyl |
| A-34 | 2,4,6-trimethylphenyl | isopropyl | phenyl |
| A-35 | 2,4,6-trimethylphenyl | n-butyl | phenyl |
| A-36 | 2,4,6-trimethylphenyl | isobutyl | phenyl |
| A-37 | 2,4,6-trimethylphenyl | sec-buty | phenyl |
| A-38 | 2,4,6-trimethylphenyl | tert-butyl | phenyl |
| A-39 | 2,4,6-trimethylphenyl | cyclohexyl | phenyl |
| A-40 | 2,4,6-trimethylphenyl | 1-adamantyl | phenyl |
| A-41 | 2,6-diisopropylphenyl | methyl | H |
| A-42 | 2,6-diisopropylphenyl | ethyl | H |
| A-43 | 2,6-diisopropylphenyl | n-propyl | H |
| A-44 | 2,6-diisopropylphenyl | isopropyl | H |
| A-45 | 2,6-diisopropylphenyl | n-butyl | H |
| A-46 | 2,6-diisopropylphenyl | isobutyl | H |
| A-47 | 2,6-diisopropylphenyl | sec-buty | H |
| A-48 | 2,6-diisopropylphenyl | tert-butyl | H |
| A-49 | 2,6-diisopropylphenyl | cyclohexyl | H |
| A-50 | 2,6-diisopropylphenyl | 1-adamantyl | H |
| A-51 | 2,6-diisopropylphenyl | methyl | phenyl |
| A-52 | 2,6-diisopropylphenyl | ethyl | phenyl |
| A-53 | 2,6-diisopropylphenyl | n-propyl | phenyl |
| A-54 | 2,6-diisopropylphenyl | isopropyl | phenyl |
| A-55 | 2,6-diisopropylphenyl | n-butyl | phenyl |
| A-56 | 2,6-diisopropylphenyl | isobutyl | phenyl |
| A-57 | 2,6-diisopropylphenyl | sec-buty | phenyl |
| A-58 | 2,6-diisopropylphenyl | tert-butyl | phenyl |
| A-59 | 2,6-diisopropylphenyl | cyclohexyl | phenyl |
| A-60 | 2,6-diisopropylphenyl | 1-adamantyl | phenyl |
| A-61 | (2-chloro-6- | methyl | H |
| | methyl)phenyl | | |
| A-62 | (2-chloro-6- | ethyl | H |
| | methyl)phenyl | | |
| A-63 | (2-chloro-6- | n-propyl | H |
| | methyl)phenyl | | |
| A-64 | (2-chloro-6- | isopropyl | H |
| | methyl)phenyl | | |
| A-65 | (2-chloro-6- | n-butyl | H |
| | methyl)phenyl | | |
| A-66 | (2-chloro-6- | isobutyl | H |
| | methyl)phenyl | | |
| A-67 | (2-chloro-6- | sec-buty | H |
| | methyl)phenyl | | |
| A-68 | (2-chloro-6- | tert-butyl | H |
| | methyl)phenyl | | |
| A-69 | (2-chloro-6- | cyclohexyl | H |
| | methyl)phenyl | | |
| A-70 | (2-chloro-6- | 1-adamantyl | H |
| | methyl)phenyl | | |
| A-71 | (2-chloro-6- | methyl | phenyl |
| | methyl)phenyl | | |
| A-72 | (2-chloro-6- | ethyl | phenyl |
| | methyl)phenyl | | |
| A-73 | (2-chloro-6- | n-propyl | phenyl |
| | methyl)phenyl | | |
| A-74 | (2-chloro-6- | isopropyl | phenyl |
| | methyl)phenyl | | |
| A-75 | (2-chloro-6- | n-butyl | phenyl |
| | methyl)phenyl | | |
| A-76 | (2-chloro-6- | isobutyl | phenyl |
| | methyl)phenyl | | |
| A-77 | (2-chloro-6- | sec-buty | phenyl |
| | methyl)phenyl | | |
| A-78 | (2-chloro-6- | tert-butyl | phenyl |
| | methyl)phenyl | | |
| A-79 | (2-chloro-6- | cyclohexyl | phenyl |
| | methyl)phenyl | | |
| A-80 | (2-chloro-6- | 1-adamantyl | phenyl |
| | methyl)phenyl | | |
| A-81 | tert-butyl | methyl | H |
| A-82 | tert-butyl | ethyl | H |
| A-83 | tert-butyl | n-propyl | H |
| A-84 | tert-butyl | isopropyl | H |
| A-85 | tert-butyl | n-butyl | H |
| A-86 | tert-butyl | isobutyl | H |
| A-87 | tert-butyl | sec-buty | H |
| A-88 | tert-butyl | tert-butyl | H |
| A-89 | tert-butyl | cyclohexyl | H |
| A-90 | tert-butyl | 1-adamantyl | H |
| A-91 | tert-butyl | methyl | phenyl |
| A-92 | tert-butyl | ethyl | phenyl |
| A-93 | tert-butyl | n-propyl | phenyl |
| A-94 | tert-butyl | isopropyl | phenyl |
| A-95 | tert-butyl | n-butyl | phenyl |
| A-96 | tert-butyl | isobutyl | phenyl |
| A-97 | tert-butyl | sec-buty | phenyl |
| A-98 | tert-butyl | tert-butyl | phenyl |
| A-99 | tert-butyl | cyclohexyl | phenyl |
| A-100 | tert-butyl | 1-adamantyl | phenyl |

In a preferred embodiment of the aforementioned second variant, the process of the invention is employed for preparing compounds of the general formula (I-B.1) or (I-B.2)

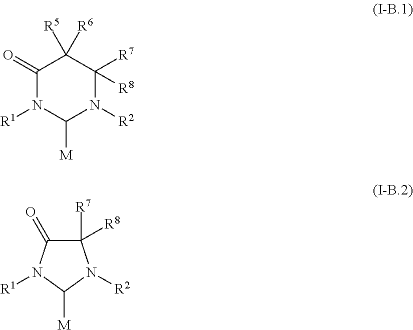

where M, R¹, R², R⁵, R⁶, R⁷ and R⁸ have the aforementioned meanings, in particular the meanings mentioned as preferred.

In an especially preferred embodiment of the second variant, the process of the invention is employed for preparing compounds of the general formula (I-B.2.1)

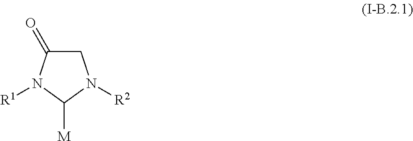

where M, R¹ and R² have the aforementioned meanings, in particular the meanings mentioned as preferred.

The compounds of the general formula (I-B.2.1) which are indicated in Tables 6 to 10 below represent certain embodiments of the present invention. The meanings for R¹ and R² indicated in Table B below represent embodiments of the invention which are like-wise preferred independently of one another and especially in combination.

### Table 6

Compounds of the formula (I-B.2.1) in which the group M is PdCl₂(CNR¹) and the combination of R¹ and R² for a compound in each case corresponds to one line of Table B. The residue R¹ bound to the ring nitrogen atom and the residue R¹ in the group PdCl₂(CNR¹) have both the same meaning. In comparison to the general formula (I), in formula (I-B.2.1) n is 0 and the residues R⁷ and R⁸ are hydrogen and are not depicted.

### Table 7

Compounds of the formula (I-B.2.1) in which the group M is PtCl₂(CNR¹) and the combination of R¹ and R² for a compound in each case corresponds to one line of Table B. The residue R¹ bound to the ring nitrogen atom and the residue R¹ in the group PtCl₂(CNR¹) have both the same meaning.

### Table 8

Compounds of the formula (I-B.2.1) in which the group M is PdCl(CNR¹)₂ and the combination of R¹, R² and R³ for a compound in each case corresponds to one line of Table B. The residue R¹ bound to the ring nitrogen atom and the residue R¹ in the group PdCl (CNR¹)₂ have both the same meaning.

### Table 9

Compounds of the formula (I-B.2.1) in which the group M is AuCl and the combination of R¹ and R² for a compound in each case corresponds to one line of Table B.

### Table 10

Compounds of the formula (I-B.2.1) in which the group M is Au(CNR¹) and the combination of R¹ and R² for a compound in each case corresponds to one line of Table B. The residue R¹ bound to the ring nitrogen atom and the residue R¹ in the group AuCl(CNR¹) have both the same meaning.

TABLE B

| No. | R¹ | R² |
|---|---|---|
| B-1 | 2,6-dimethylphenyl | isopropyl |
| B-2 | 2,6-dimethylphenyl | tert-butyl |
| B-3 | 2,6-dimethylphenyl | cyclohexyl |
| B-4 | 2,6-dimethylphenyl | cyclododecyl |
| B-5 | 2,6-dimethylphenyl | 1-adamantyl |
| B-6 | 2,6-dimethylphenyl | 1- |
| | | phenylethyl |
| B-7 | 2,4,6-trimethylphenyl | isopropyl |
| B-8 | 2,4,6-trimethylphenyl | tert-butyl |
| B-9 | 2,4,6-trimethylphenyl | cyclohexyl |
| B-10 | 2,4,6-trimethylphenyl | cyclododecyl |
| B-11 | 2,4,6-trimethylphenyl | 1-adamantyl |
| B-12 | 2,4,6-trimethylphenyl | 1- |
| | | phenylethyl |
| B-13 | 2,6-diisopropylphenyl | isopropyl |
| B-14 | 2,6-diisopropylphenyl | tert-butyl |
| B-15 | 2,6-diisopropylphenyl | cyclohexyl |
| B-16 | 2,6-diisopropylphenyl | cyclododecyl |
| B-17 | 2,6-diisopropylphenyl | 1-adamantyl |
| B-18 | 2,6-diisopropylphenyl | 1- |
| | | phenylethyl |
| B-19 | (2-chloro-6- | isopropyl |
| | methyl)phenyl | |
| B-20 | (2-chloro-6- | tert-butyl |
| | methyl)phenyl | |
| B-21 | (2-chloro-6- | cyclohexyl |
| | methyl)phenyl | |
| B-22 | (2-chloro-6- | cyclododecyl |
| | methyl)phenyl | |
| B-23 | (2-chloro-6- | 1-adamantyl |
| | methyl)phenyl | |
| B-24 | (2-chloro-6- | 1- |
| | methyl)phenyl | phenylethyl |
| B-25 | tert-butyl | isopropyl |
| B-26 | tert-butyl | tert-butyl |
| B-27 | tert-butyl | cyclohexyl |
| B-28 | tert-butyl | cyclododecyl |
| B-29 | tert-butyl | 1-adamantyl |
| B-30 | tert-butyl | 1- |
| | | phenylethyl |

In a preferred embodiment of the third variant, the process of the invention is employed for preparing compounds of the general formula (I-C.1) or (I-C.2)

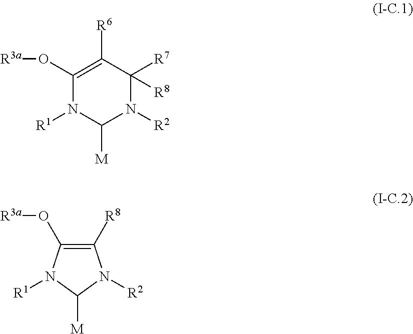

where
M, R¹, R², R^{3a}, R⁶, R⁷ and R⁸ have the aforementioned meanings, in particular the meanings mentioned as preferred.

In an especially preferred embodiment of the third variant, the process of the invention is employed for preparing compounds of the general formula (I-C.2.1)

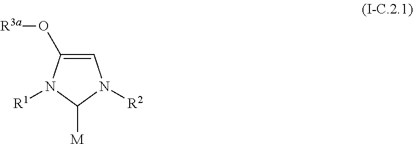

where M, R¹, R² and R^{3a} have the aforementioned meanings, in particular the meanings mentioned as preferred.

The compounds of the general formula (I-C.2.1) which are indicated in Tables 11 to 15 below represent certain embodiments of the present invention. The meanings for R¹, R² and R^{3a} indicated in Table C below represent embodiments of the invention which are likewise preferred independently of one another and especially in combination.

### Table 10

Compounds of the formula (I-C.2.1) in which the group M is PdCl₂(CNR¹) and the combination of R¹, R² and R^{3a} for a compound in each case corresponds to one line of Table C. The residue R¹ bound to the ring nitrogen atom and the residue R¹ in the group PdCl₂(CNR¹) have both the same meaning. In comparison to the general formula (I), in formula (I-C.2.1) n is 0 and the residue R⁸ is hydrogen and is not depicted.

### Table 11

Compounds of the formula (I-C.2.1) in which the group M is PtCl₂(CNR¹) and the combination of R¹, R² and R^{3a} for a compound in each case corresponds to one line of Table C. The residue R¹ bound to the ring nitrogen atom and the residue R¹ in the group PtCl₂(CNR¹) have both the same meaning.

### Table 12

Compounds of the formula (I-C.2.1) in which the group M is PdCl(CNR¹)₂ and the combination of R¹, R² and R³ for a compound in each case corresponds to one line of Table C. The residue R¹ bound to the ring nitrogen atom and the residue R¹ in the group PdCl(CNR¹)₂ have both the same meaning.

### Table 14

Compounds of the formula (I-C.2.1) in which the group M is AuCl and the combination of R¹, R² and R^{3a} for a compound in each case corresponds to one line of Table C.

### Table 15

Compounds of the formula (I-C.2.1) in which the group M is Au(CNR¹) and the combination of R¹, R² and R^{3a} for a compound in each case corresponds to one line of Table C. The residue R¹ bound to the ring nitrogen atom and the residue R¹ in the group Au(CNR¹) have both the same meaning.

TABLE C

| No. | R¹ | R² | R^{3a} |
|---|---|---|---|
| C-1 | 2,6-dimethylphenyl | isopropyl | tert-butyldiphenylsilyl |
| C-2 | 2,6-dimethylphenyl | tert-butyl | tert-butyldiphenylsilyl |
| C-3 | 2,6-dimethylphenyl | cyclohexyl | tert-butyldiphenylsilyl |
| C-4 | 2,6-dimethylphenyl | cyclododecyl | tert-butyldiphenylsilyl |
| C-5 | 2,6-dimethylphenyl | 1-adamantyl | tert-butyldiphenylsilyl |
| C-6 | 2,6-dimethylphenyl | 1- | tert-butyldiphenylsilyl |
| | | phenylethyl | |
| C-7 | 2,6-dimethylphenyl | isopropyl | benzoyl |
| C-8 | 2,6-dimethylphenyl | tert-butyl | benzoyl |
| C-9 | 2,6-dimethylphenyl | cyclohexyl | benzoyl |
| C-10 | 2,6-dimethylphenyl | cyclododecyl | benzoyl |
| C-11 | 2,6-dimethylphenyl | 1-adamantyl | benzoyl |
| C-12 | 2,6-dimethylphenyl | 1- | benzoyl |
| | | phenylethyl | |
| C-13 | 2,6-dimethylphenyl | isopropyl | 4,5-dimethoxy-2- |
| | | | nitrobenzyloxycarbonyl |
| C-14 | 2,6-dimethylphenyl | tert-butyl | 4,5-dimethoxy-2- |
| | | | nitrobenzyloxycarbonyl |
| C-15 | 2,6-dimethylphenyl | cyclohexyl | 4,5-dimethoxy-2- |
| | | | nitrobenzyloxycarbonyl |
| C-16 | 2,6-dimethylphenyl | cyclododecyl | 4,5-dimethoxy-2- |
| | | | nitrobenzyloxycarbonyl |
| C-17 | 2,6-dimethylphenyl | 1-adamantyl | 4,5-dimethoxy-2- |
| | | | nitrobenzyloxycarbonyl |
| C-18 | 2,6-dimethylphenyl | 1- | 4,5-dimethoxy-2- |
| | | phenylethyl | nitrobenzyloxycarbonyl |
| C-19 | 2,6-diisopropylphenyl | isopropyl | tert-butyldiphenylsilyl |
| C-20 | 2,6-diisopropylphenyl | tert-butyl | tert-butyldiphenylsilyl |
| C-21 | 2,6-diisopropylphenyl | cyclohexyl | tert-butyldiphenylsilyl |
| C-22 | 2,6-diisopropylphenyl | cyclododecyl | tert-butyldiphenylsilyl |
| C-23 | 2,6-diisopropylphenyl | 1-adamantyl | tert-butyldiphenylsilyl |
| C-24 | 2,6-diisopropylphenyl | 1- | tert-butyldiphenylsilyl |
| | | phenylethyl | |
| C-25 | 2,6-diisopropylphenyl | isopropyl | benzoyl |
| C-26 | 2,6-diisopropylphenyl | tert-butyl | benzoyl |
| C-27 | 2,6-diisopropylphenyl | cyclohexyl | benzoyl |
| C-28 | 2,6-diisopropylphenyl | cyclododecyl | benzoyl |
| C-29 | 2,6-diisopropylphenyl | 1-adamantyl | benzoyl |
| C-30 | 2,6-diisopropylphenyl | 1- | benzoyl |
| | | phenylethyl | |
| C-31 | 2,6-diisopropylphenyl | isopropyl | 4,5-dimethoxy-2- |
| | | | nitrobenzyloxycarbonyl |
| C-32 | 2,6-diisopropylphenyl | tert-butyl | 4,5-dimethoxy-2- |
| | | | nitrobenzyloxycarbonyl |
| C-33 | 2,6-diisopropylphenyl | cyclohexyl | 4,5-dimethoxy-2- |
| | | | nitrobenzyloxycarbonyl |
| C-34 | 2,6-diisopropylphenyl | cyclododecyl | 4,5-dimethoxy-2- |
| | | | nitrobenzyloxycarbonyl |
| C-35 | 2,6-diisopropylphenyl | 1-adamantyl | 4,5-dimethoxy-2- |
| | | | nitrobenzyloxycarbonyl |
| C-36 | 2,6-diisopropylphenyl | 1- | 4,5-dimethoxy-2- |
| | | phenylethyl | nitrobenzyloxycarbonyl |
| C-37 | (2-chloro-6- | isopropyl | tert-butyldiphenylsilyl |
| | methyl)phenyl | | |
| C-38 | (2-chloro-6- | tert-butyl | tert-butyldiphenylsilyl |
| | methyl)phenyl | | |
| C-39 | (2-chloro-6- | cyclohexyl | tert-butyldiphenylsilyl |
| | methyl)phenyl | | |
| C-40 | (2-chloro-6- | cyclododecyl | tert-butyldiphenylsilyl |
| | methyl)phenyl | | |
| C-41 | (2-chloro-6- | 1-adamantyl | tert-butyldiphenylsilyl |
| | methyl)phenyl | | |
| C-42 | (2-chloro-6- | 1- | tert-butyldiphenylsilyl |
| | methyl)phenyl | phenylethyl | |
| C-43 | (2-chloro-6- | isopropyl | benzoyl |
| | methyl)phenyl | | |
| C-44 | (2-chloro-6- | tert-butyl | benzoyl |
| | methyl)phenyl | | |
| C-45 | (2-chloro-6- | cyclohexyl | benzoyl |
| | methyl)phenyl | | |
| C-46 | (2-chloro-6- | cyclododecyl | benzoyl |
| | methyl)phenyl | | |
| C-47 | (2-chloro-6- | 1-adamantyl | benzoyl |
| | methyl)phenyl | | |
| C-48 | (2-chloro-6- | 1- | benzoyl |
| | methyl)phenyl | phenylethyl | |
| C-49 | (2-chloro-6- | isopropyl | 4,5-dimethoxy-2- |
| | methyl)phenyl | | nitrobenzyloxycarbonyl |
| C-50 | (2-chloro-6- | tert-butyl | 4,5-dimethoxy-2- |
| | methyl)phenyl | | nitrobenzyloxycarbonyl |
| C-51 | (2-chloro-6- | cyclohexyl | 4,5-dimethoxy-2- |
| | methyl)phenyl | | nitrobenzyloxycarbonyl |
| C-52 | (2-chloro-6- | cyclododecyl | 4,5-dimethoxy-2- |
| | methyl)phenyl | | nitrobenzyloxycarbonyl |
| C-53 | (2-chloro-6- | 1-adamantyl | 4,5-dimethoxy-2- |
| | methyl)phenyl | | nitrobenzyloxycarbonyl |
| C-54 | (2-chloro-6- | 1- | 4,5-dimethoxy-2- |
| | methyl)phenyl | phenylethyl | nitrobenzyloxycarbonyl |
| C-55 | tert-butyl | isopropyl | tert-butyldiphenylsilyl |
| C-56 | tert-butyl | tert-butyl | tert-butyldiphenylsilyl |
| C-57 | tert-butyl | cyclohexyl | tert-butyldiphenylsilyl |
| C-58 | tert-butyl | cyclododecyl | tert-butyldiphenylsilyl |
| C-59 | tert-butyl | 1-adamantyl | tert-butyldiphenylsilyl |
| C-60 | tert-butyl | 1- | tert-butyldiphenylsilyl |
| | | phenylethyl | |
| C-61 | tert-butyl | isopropyl | benzoyl |
| C-62 | tert-butyl | tert-butyl | benzoyl |
| C-63 | tert-butyl | cyclohexyl | benzoyl |
| C-64 | tert-butyl | cyclododecyl | benzoyl |
| C-65 | tert-butyl | 1-adamantyl | benzoyl |
| C-66 | tert-butyl | 1- | benzoyl |
| | | phenylethyl | |
| C-67 | tert-butyl | isopropyl | 4,5-dimethoxy-2- |
| | | | nitrobenzyloxycarbonyl |
| C-68 | tert-butyl | tert-butyl | 4,5-dimethoxy-2- |
| | | | nitrobenzyloxycarbonyl |
| C-69 | tert-butyl | cyclohexyl | 4,5-dimethoxy-2- |
| | | | nitrobenzyloxycarbonyl |
| C-70 | tert-butyl | cyclododecyl | 4,5-dimethoxy-2- |
| | | | nitrobenzyloxycarbonyl |
| C-71 | tert-butyl | 1-adamantyl | 4,5-dimethoxy-2- |
| | | | nitrobenzyloxycarbonyl |
| C-72 | tert-butyl | 1- | 4,5-dimethoxy-2- |
| | | phenylethyl | nitrobenzyloxycarbonyl |

In a preferred embodiment of the second aspect, the process of the invention is employed for preparing compounds of the general formula I-E,

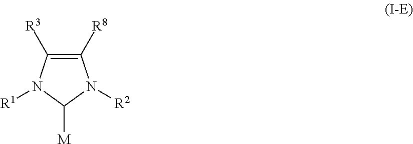

where M, R¹, R², R³ and R⁸ have the aforementioned meanings. In this embodiment, R¹ and R² have preferably different meanings.

R¹ is preferably selected from groups of the formulae IV.1 to IV.5, with particular preference given to the formulae IV.1 and IV.2. More preferably, R¹ is selected from C₁-C₆-alkyl, phenyl and phenyl which carries 1, 2 or 3 radicals independently selected from C₁-C₆-alkyl and chlorine. In particular preferably, R¹ is selected from tert.-butyl, 2,6-dimethylphenyl, 2,4,6-trimethylphenyl, 2,6-diisopropylphenyl and (2-chloro-6-methyl)phenyl.

Preferably, R² is selected from alkyl and cycloalkyl, in particular C₁-C₆-alkyl, phenyl-C₁-C₆-alkyl and C₅-C₁₅-cycloalkyl. In particular, R² is selected from methyl, ethyl, n-propyl, isopropyl, n-butyl, sec-butyl, tert-butyl, isobutyl, cyclopentyl, cyclohexyl, cyclododecyl and 1-adamantyl.

Preferably, R³ is selected from hydrogen, alkyl, cycloalkyl and aryl, more preferably hydrogen, C₁-C₆-alkyl and C₆-C₁₀-aryl.

Preferably, R⁸ is selected from hydrogen, alkyl, cycloalkyl and aryl, more preferably hydrogen, C₁-C₆-alkyl and C₆-C₁₀-aryl.

M is preferably selected from PdCl₂(CNR¹), PtCl₂(CNR¹), PdCl(CNR¹)₂, Au (CNR¹) and AuCl, where R¹ is selected from hydrogen, alkyl, cycloalkyl, heterocycloalkyl, aryl and hetaryl.

In an especially preferred embodiment of the second aspect, the process of the invention is employed for preparing compounds of the formula I-E.1,

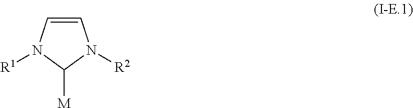

where M, R¹ and R² have the aforementioned meanings, in particular the meanings mentioned as preferred.

The compounds of the general formula (I-E.1) which are indicated in Tables 16 to 20 below represent certain embodiments of the present invention. The meanings for R¹ and R² indicated in Table D below represent embodiments of the invention which are like-wise preferred independently of one another and especially in combination.

### Table 16

Compounds of the formula (I-E.1) in which the group M is PdCl₂(CNR¹) and the combination of R¹ and R² for a compound in each case corresponds to one line of Table D. The residue R¹ bound to the ring nitrogen atom and the residue R¹ in the group PdCl₂(CNR¹) have both the same meaning.

### Table 17

Compounds of the formula (I-E.1) in which the group M is PtCl₂(CNR¹) and the combination of R¹ and R² for a compound in each case corresponds to one line of Table D.

The residue R¹ bound to the ring nitrogen atom and the residue R¹ in the group PtCl₂(CNR¹) have both the same meaning.

### Table 18

Compounds of the formula (I-E.1) in which the group M is PdCl(CNR¹)₂ and the combination of R¹ and R² for a compound in each case corresponds to one line of Table D. The residue R¹ bound to the ring nitrogen atom and the residue R¹ in the group PdCl(CNR¹)₂ have both the same meaning.

### Table 19

Compounds of the formula (I-E.1) in which the group M is AuCl and the combination of R¹ and R² for a compound in each case corresponds to one line of Table D.

### Table 20

Compounds of the formula (I-E.1) in which the group M is Au(CNR¹) and the combination of R¹ and R² for a compound in each case corresponds to one line of Table D. he residue R¹ bound to the ring nitrogen atom and the residue R¹ in the group Au(CNR¹) have both the same meaning.

TABLE D

| No. | R¹ | R² |
|---|---|---|
| D-1 | 2,6-dimethylphenyl | isopropyl |
| D-2 | 2,6-dimethylphenyl | tert-butyl |
| D-3 | 2,6-dimethylphenyl | cyclohexyl |
| D-4 | 2,6-dimethylphenyl | cyclododecyl |
| D-5 | 2,6-dimethylphenyl | 1-adamantyl |
| D-6 | 2,6-dimethylphenyl | 1- |
| | | phenylethyl |
| D-7 | 2,4,6-trimethylphenyl | isopropyl |
| D-8 | 2,4,6-trimethylphenyl | tert-butyl |
| D-9 | 2,4,6-trimethylphenyl | cyclohexyl |
| D-10 | 2,4,6-trimethylphenyl | cyclododecyl |
| D-11 | 2,4,6-trimethylphenyl | 1-adamantyl |
| D-12 | 2,4,6-trimethylphenyl | 1- |
| | | phenylethyl |
| D-13 | 2,6-diisopropylphenyl | isopropyl |
| D-14 | 2,6-diisopropylphenyl | tert-butyl |
| D-15 | 2,6-diisopropylphenyl | cyclohexyl |
| D-16 | 2,6-diisopropylphenyl | cyclododecyl |
| D-17 | 2,6-diisopropylphenyl | 1-adamantyl |
| D-18 | 2,6-diisopropylphenyl | 1- |
| | | phenylethyl |
| D-19 | (2-chloro-6- | isopropyl |
| | methyl)phenyl | |
| D-20 | (2-chloro-6- | tert-butyl |
| | methyl)phenyl | |
| D-21 | (2-chloro-6- | cyclohexyl |
| | methyl)phenyl | |
| D-22 | (2-chloro-6- | cyclododecyl |
| | methyl)phenyl | |
| D-23 | (2-chloro-6- | 1-adamantyl |
| | methyl)phenyl | |
| D-24 | (2-chloro-6- | 1- |
| | methyl)phenyl | phenylethyl |
| D-25 | tert-butyl | isopropyl |
| D-26 | tert-butyl | tert-butyl |
| D-27 | tert-butyl | cyclohexyl |
| D-28 | tert-butyl | cyclododecyl |
| D-29 | tert-butyl | 1-adamantyl |
| D-30 | tert-butyl | 1- |
| | | phenylethyl |

In a preferred embodiment of the third aspect, the process according to the invention is employed for preparing compounds of the general formula I-F

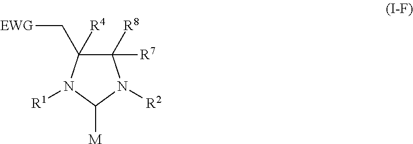

where M, EWG, R¹, R², R⁴, R⁷ and R⁸ have the aforementioned meanings. In this embodiment, R¹ and R² have preferably different meanings.

In this embodiment, R¹ is preferably selected from phenyl, naphthyl, phenyl which carries 1, 2 or 3 radicals independently selected from C₁-C₈-alkyl and C₁-C₈-haloalkyl, and naphthyl which carries 1, 2 or 3 radicals independently selected from C₁-C₈-alkyl and C₁-C₈-haloalkyl, in particular C₁-C₆-alkyl or C₁-C₆-fluoroalkyl. Especially R¹ is selected from 2,6-dimethylphenyl, 2,4,6-trimethylphenyl, 2,6-diisopropylphenyl, 2-trifluoromethylphenyl, 2,6-di-(trifluoromethyl)phenyl, 1-napthyl and 2-naphthyl.

Preferably, R² is selected from alkyl and cycloalkyl, in particular C₁-C₆-alkyl, phenyl-C₁-C₆-alkyl and C₅-C₁₅-cycloalkyl. More preferably, R² is selected from methyl, ethyl, n-propyl, isopropyl, n-butyl, sec-butyl, tert-butyl, isobutyl, 1-phenylethyl, cyclopentyl, cyclohexyl, cyclododecyl and 1-adamantyl.

Preferably, R⁴ is selected from hydrogen, alkyl, cycloalkyl and aryl, more preferably hydrogen and C₁-C₁₀-alkyl.

Preferably, R⁷ and R⁸ are independently of each other selected from hydrogen, alkyl, cycloalkyl and aryl, more preferably hydrogen and C₁-C₁₀-alkyl.

EWG is preferably C(O)R¹⁴ or C(O)OR¹⁴, especially C(O)OR¹⁴. In this embodiment R¹⁴ is preferably C₁-C₆-alkyl, especially C₁-C₄-alkyl such as methyl, ethyl, n-propyl, iso-propyl, n-butyl, sec-butyl, isobutyl or tert-butyl.

M is preferably selected from PdCl₂(CNR¹), PtCl₂(CNR¹), PdCl(CNR¹)₂, Au (CNR¹) and AuCl, where R¹ is selected from hydrogen, alkyl, cycloalkyl, heterocycloalkyl, aryl and hetaryl.

Preference is given to compounds of formula I-F.1,

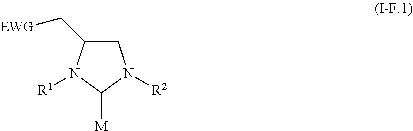

where EWG, M, R¹ and R² have the aforementioned meanings, preferably those being preferred.

The compounds of the general formula (I-F.1) which are indicated in Tables 21 to 30 below represent certain embodiments of the present invention. The meanings for R¹ and R² indicated in Table E below represent embodiments of the invention which are like-wise preferred independently of one another and especially in combination.

### Table 21

Compounds of the formula (I-F.1) in which the group M is PdCl₂(CNR¹), EWG is methoxycarbonyl and the combination of R¹ and R² for a compound in each case corresponds to one line of Table E. The residue R¹ bound to the ring nitrogen atom and the residue R¹ in the group PdCl₂(CNR¹) have both the same meaning.

### Table 22

Compounds of the formula (I-F.1) in which the group M is PtCl₂(CNR¹), EWG is methoxycarbonyl and the combination of R¹ and R² for a compound in each case corresponds to one line of Table E. The residue R¹ bound to the ring nitrogen atom and the residue R¹ in the group PtCl₂(CNR¹) have both the same meaning.

### Table 23

Compounds of the formula (I-F.1) in which the group M is PdCl(CNR¹)₂, EWG is methoxycarbonyl and the combination of R¹ and R² for a compound in each case corresponds to one line of Table E. The residue R¹ bound to the ring nitrogen atom and the residue R¹ in the group PdCl(CNR¹)₂ have both the same meaning.

### Table 24

Compounds of the formula (I-F.1) in which the group M is AuCl, EWG is methoxycarbonyl and the combination of R¹ and R² for a compound in each case corresponds to one line of Table E.

### Table 25

Compounds of the formula (I-F.1) in which the group M is Au(CNR¹), EWG is methoxycarbonyl and the combination of R¹ and R² for a compound in each case corresponds to one line of Table E. he residue R¹ bound to the ring nitrogen atom and the residue R¹ in the group Au(CNR¹) have both the same meaning.

### Table 26

Compounds of the formula (I-F.1) in which the group M is PdCl₂(CNR¹), EWG is ethoxycarbonyl and the combination of R¹ and R² for a compound in each case corresponds to one line of Table E. The residue R¹ bound to the ring nitrogen atom and the residue R¹ in the group PdCl₂(CNR¹) have both the same meaning.

### Table 27

Compounds of the formula (I-F.1) in which the group M is PtCl₂(CNR¹), EWG is ethoxycarbonyl and the combination of R¹ and R² for a compound in each case corresponds to one line of Table E. The residue R¹ bound to the ring nitrogen atom and the residue R¹ in the group PtCl₂(CNR¹) have both the same meaning.

### Table 28

Compounds of the formula (I-F.1) in which the group M is PdCl(CNR¹)₂, EWG is ethoxycarbonyl and the combination of R¹ and R² for a compound in each case corresponds to one line of Table E. The residue R¹ bound to the ring nitrogen atom and the residue R¹ in the group PdCl(CNR¹)₂ have both the same meaning.

### Table 29

Compounds of the formula (I-F.1) in which the group M is AuCl, EWG is ethoxycarbonyl and the combination of R¹ and R² for a compound in each case corresponds to one line of Table E.

### Table 30

Compounds of the formula (I-F.1) in which the group M is Au(CNR¹), EWG is ethoxycarbonyl and the combination of R¹ and R² for a compound in each case corresponds to one line of Table E. he residue R¹ bound to the ring nitrogen atom and the residue R¹ in the group Au(CNR¹) have both the same meaning.

TABLE E

| No. | R¹ | R² |
|---|---|---|
| E-1 | 2,6-dimethylphenyl | isopropyl |
| E-2 | 2,6-dimethylphenyl | tert-butyl |
| E-3 | 2,6-dimethylphenyl | cyclohexyl |
| E-4 | 2,6-dimethylphenyl | cyclododecyl |
| E-5 | 2,6-dimethylphenyl | 1-adamantyl |
| E-6 | 2,6-dimethylphenyl | 1- |
| | | phenylethyl |
| E-7 | 2,4,6-trimethylphenyl | isopropyl |
| E-8 | 2,4,6-trimethylphenyl | tert-butyl |
| E-9 | 2,4,6-trimethylphenyl | cyclohexyl |
| E-10 | 2,4,6-trimethylphenyl | cyclododecyl |
| E-11 | 2,4,6-trimethylphenyl | 1-adamantyl |
| E-12 | 2,4,6-trimethylphenyl | 1- |
| | | phenylethyl |
| E-13 | 2,6-diisopropylphenyl | isopropyl |
| E-14 | 2,6-diisopropylphenyl | tert-butyl |
| E-15 | 2,6-diisopropylphenyl | cyclohexyl |
| E-16 | 2,6-diisopropylphenyl | cyclododecyl |
| E-17 | 2,6-diisopropylphenyl | 1-adamantyl |
| E-18 | 2,6-diisopropylphenyl | 1- |
| | | phenylethyl |
| E-19 | (2-trifluromethyl)phenyl | isopropyl |
| E-20 | (2-trifluromethyl)phenyl | tert-butyl |
| E-21 | (2-trifluromethyl)phenyl | cyclohexyl |
| E-22 | (2-trifluromethyl)phenyl | cyclododecyl |
| E-23 | (2-trifluromethyl)phenyl | 1-adamantyl |
| E-24 | (2-trifluromethyl)phenyl | 1- |
| | | phenylethyl |
| E-25 | 1-naphthyl | isopropyl |
| E-26 | 1-naphthyl | tert-butyl |
| E-27 | 1-naphthyl | cyclohexyl |
| E-28 | 1-naphthyl | cyclododecyl |
| E-29 | 1-naphthyl | 1-adamantyl |
| E-30 | 1-naphthyl | 1- |
| | | phenylethyl |
| E-31 | 2-naphthyl | isopropyl |
| E-32 | 2-naphthyl | tert-butyl |
| E-33 | 2-naphthyl | cyclohexyl |
| E-34 | 2-naphthyl | cyclododecyl |
| E-35 | 2-naphthyl | 1-adamantyl |
| E-36 | 2-naphthyl | 1- |
| | | phenylethyl |

Step a1)

In step a1) of the process according to the invention an isonitrile complex of the general formula (II) R¹—N≡C-M is employed, wherein R¹ and M have one of the meanings given above.

### Synthesis of Isonitrile Ligands

The syntheses of the isonitriles suitable for providing isonitrile complexes of the general formula (II) can be accomplished by conventional methods, which are briefly outlined below, from precursors which are commercially available or can be obtained by known methods. Suitable methods for the formation of isonitriles are e.g. the elimination of water from N-substituted formamides, the reaction between primary amines and chloroform under basic conditions (haloform-isocyanide transformation), and the reduction of isocyanates. A preferred method to provide isonitriles of the general formula R¹—N≡C is the reaction of an amine R¹—NH₂ with a formic acid ester at elevated temperature to obtain a formamide of the formula R¹—NH—CH(═O) and the subsequent elimination of water, e.g. with POCl₃ and a tertiary amine, phosgene and a tertiary amine, etc., as depicted in Scheme 1.

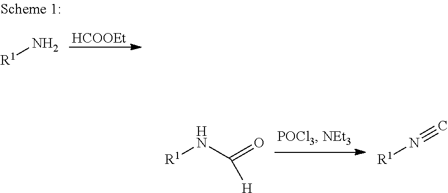

In Scheme, 1 R¹ has the aforementioned meanings, in particular the meanings mentioned as preferred, and Et means ethyl.

### Synthesis of Isonitrile Complexes of the General Formula (II)

In the process of the invention the desired metal carbene complexes are prepared from metal coordinated isonitrile ligands (isocyanide ligands). The employed isonitrile complexes of the general formula (II) R¹—N≡C-M can be obtained e.g. from the aforementioned isonitrile compounds R¹—N≡C and readily available complexes of a metal M by ligand exchange reactions known to a person skilled in the art as depicted in Scheme 2. Preferred educt complexes are e.g. [Pd(CH₃CN)₂Cl₂], [Pt(CH₃CN)₂Cl₂] and [AuCl(tetrahydrothiophene)].

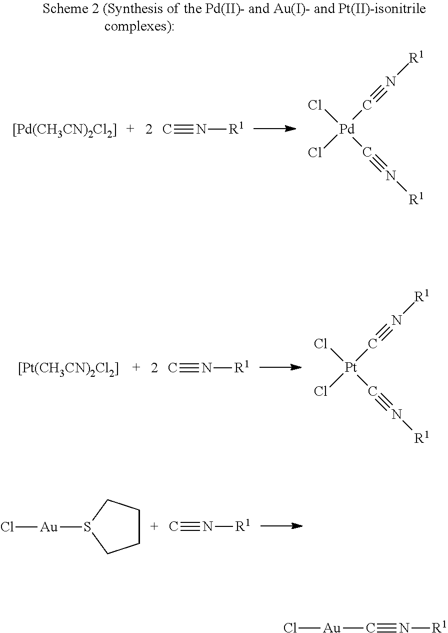

In Scheme 2, R¹ has the aforementioned meanings, in particular the meanings mentioned as preferred.

### Synthesis of NHC Complexes

To obtain the desired NHC complexes, an isonitrile complex of the general formula (II) is reacted with a compound of the general formulae (III) or (IIIa)

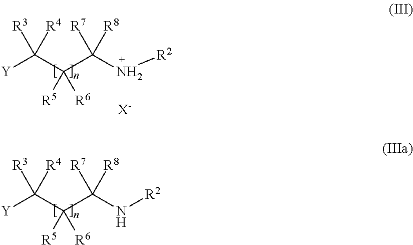

wherein
n, R², R³, R⁴, R⁵, R⁶, R⁷ and R⁸ have one of the meanings given above,
X⁻ is an anion equivalent, and
Y is a leaving group, or if R³ and R⁴ together with the carbon atom to which they are bound are C═O then Y is a group O—Y^{a}, where Y^{a} is unsubstituted or substituted alkyl, unsubstituted or substituted aryl, unsubstituted or substituted alkyl carbonyl, or unsubstituted or substituted arylcarbonyl.

The anion equivalent X⁻ serves merely as counterion and can be selected freely from among monovalent anions and the parts of polyvalent anions corresponding to a single negative charge. Suitable anions are, for example, halide ions X⁻, e.g. chloride and bromide, sulfate and sulfonate anions, e.g. SO₄²⁻, tosylate, trifluoromethanesulfonate and methanesulfonate. Preferably, X⁻ is chloride or bromide.

Preferably, Y is selected from halides, tosylates, carboxylates, carbonates, esters, sulfonates and phosphates. Examples of Y are chlorine, bromine, iodine, methanesulfonate, trifluoromethanesulfonate and toluenesulfonate, with preference given to chlorine and bromine.

Preferably, Y^{a} is selected from C₁-C₄ alkyl and pentafluorophenylcarbonyl.

In one or more embodiments, the compound of the formula (III) is an ω-haloalkylammonium salt, preferably a 2-(haloethyl)ammonium halide or a 3-(halopropyl)ammonium halide. Likewise, in a further embodiment, the compound of the formula (III.a) is an ω-haloalkylamine, preferably a 2-(haloethyl)amine or a 3-(halopropyl)amine.

In particular, the compound of the formula (III) is a 2-(chloroethyl)ammonium chloride of the formula (III.1) and the compound of the formula (III.a), is a 2-(chloroethyl)amine of the formula (III.1.a)

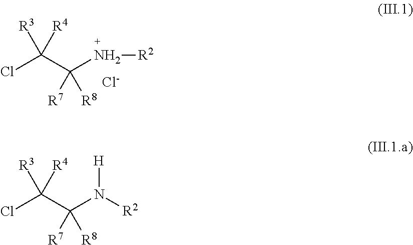

wherein
R² is selected from alkyl and cycloalkyl, in particular C₁-C₆-alkyl, phenyl-C₁-C₆-alkyl and C₅-C₁₅-cycloalkyl.
R³, R⁴, R⁷ and R⁸ are selected from hydrogen, C₁-C₆ alkyl and C₆-C₁₀ aryl.

Preferably, in the compounds (III.1) and (III.1.a), R² is selected from methyl, ethyl, n-propyl, isopropyl, n-butyl, sec-butyl, tert-butyl, cyclopentyl, cyclohexyl, cyclododecyl and 1-adamantyl.

Preferably, in the compounds (III.1) and (III.1.a), R³, R⁴, R⁷ and R⁸ are selected from hydrogen and phenyl. In a certain embodiment, R³, R⁴, R⁷ and R⁸ are all hydrogen. In a further embodiment, 1 of the residues R³, R⁴, R⁷ and R⁸ is phenyl and the other are hydrogen.

The syntheses of ω-haloalkylammonium salts (III) and the free base thereof, i.e. ω-haloalkylamin compounds (III.a) can be accomplished by conventional methods, as depicted in Scheme 3 for the ω-chloroalkylammonium salts. Thus, the synthesis of 2-(chloroethyl)ammonium chlorides can be accomplished according to literature procedures starting from commercially available amino alcohols (see e.g. A. Habtemariam et al., J. Chem. Soc. Dalton Trans. 2001, 8, 1306-1318). The free amine of the formula (III.1.a) is liberated from the salt of the formula (III.1) by addition of a base. Suitable bases are e.g. tertiary amines such as triethylamine.

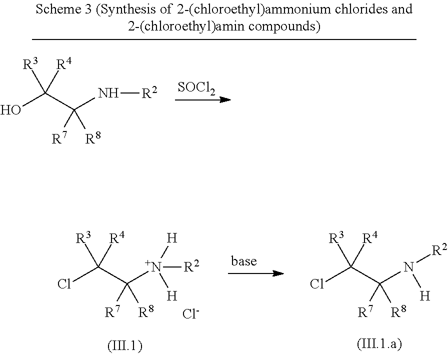

In scheme 3, R², R³, R⁴, R⁷ and R⁸ have the aforementioned meanings, in particular the meanings mentioned as preferred.

The synthesis of 2-(adamantan-1ylamino)ethanol as starting material for the formation of the corresponding 2-(chloroethyl)ammonium chloride can be performed as described by P. E. Aldrich, E. C. Herrmann, W. E. Meier, M. Paulshock, W. W. Prichard, J. A. Snyder and J. C. Watts in J. Med. Chem. 1971, 14, 535-543.

In one or more embodiments, the compound of the formula (III) is an ω-(alkoxycarbonyl)alkylammonium salt, preferably a 2-(C₁-C₄-alkoxy-carbonyl)ethylammonium halide or a 3-(C₁-C₄-alkoxycarbonyl)propylammonium halide. Likewise, in another embodiment, the compound of the formula (III.a) is an ω-(alkoxycarbonyl)alkylamine, preferably a 2-(C₁-C₄-alkoxycarbonyl)ethylamine or a 3-(C₁-C₄-alkoxycarbonyl)propylamine.

In particular, the compound of the formula (III) is a 2-(C₁-C₄-alkoxy-carbonyl)ethylammonium chloride of the formula (III.2) and the compound of the formula (III.a) is a 2-(C₁-C₄-alkoxycarbonyl)ethylamin of the formula (III.2.a)

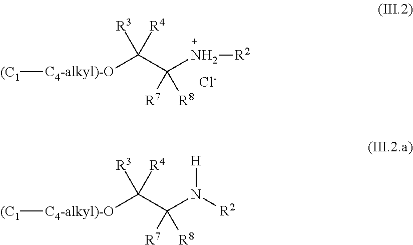

wherein
R² is selected from alkyl and cycloalkyl, in particular C₁-C₆-alkyl, phenyl-C₁-C₆-alkyl and C₅-C₁₅-cycloalkyl.
R³, R⁴, R⁷ and R⁸ are selected from hydrogen, C₁-C₆ alkyl and C₆-C₁₀ aryl.

Preferably, in the compounds (III.2) and (III.2.a), R² is selected from methyl, ethyl, n-propyl, isopropyl, n-butyl, sec-butyl, tert-butyl, cyclopentyl, cyclohexyl, cyclododecyl and 1-adamantyl.

Preferably, in the compounds (III.2) and (III.2.a), R³, R⁴, R⁷ and R⁸ are selected from hydrogen and phenyl. In a certain embodiment, R³, R⁴, R⁷ and R⁸ are all hydrogen. In a further embodiment, 1 of the residues R³, R⁴, R⁷ and R⁸ is phenyl and the others are hydrogen.

To obtain access to the desired NHC-complexes, the isonitrile complexes of the general formula (II) are reacted with a compound of the general formula (III) or (III.a).

Preferably, the reaction is performed in the presence of a base, more preferably a tertiary amine, in particular triethylamine.

Suitable reaction temperatures are generally in the range from −10 to 100° C., preferably in the range from −0 to 50° C. In a preferred embodiment, the reaction is performed at ambient temperature.

The process in step a1) of the invention can be carried out in a suitable solvent which is inert under the respective reaction conditions. Solvents which are generally suitable are, for example, aromatics such as toluene and xylenes, hydrocarbons or mixtures of hydrocarbons such as cyclohexane, ethers such as tert-butyl methyl ether, 1,4-dioxane and tetrahydrofuran, alcohols such as methanol, ethanol, n-propanol, isopropanol, n-butanol, isobutanol, ketones such as acetone and methyl ethyl ketone, etc.

Advantageously, certain embodiments of the invention show full conversions to NHC complexes (I). The reaction can be monitored e.g. by the shift of the IR-stretching frequencies of the isonitrile ligands. The method is also successful in the formation of bi-cyclic NHC complexes, e.g. by using piperidine or a derivative thereof as precursor.

Step b1)

The compounds of the general formula (I), wherein R³ and R⁴ together with the carbon atom to which they are bound are C═O and wherein the ring carbon atom adjacent to the carbonyl group bears a hydrogen atom are able to form the corresponding enol tautomers as depicted in scheme 4 for compounds (I-B.1) and (I-B.2). Those tautomers are also incorporated by the invention.

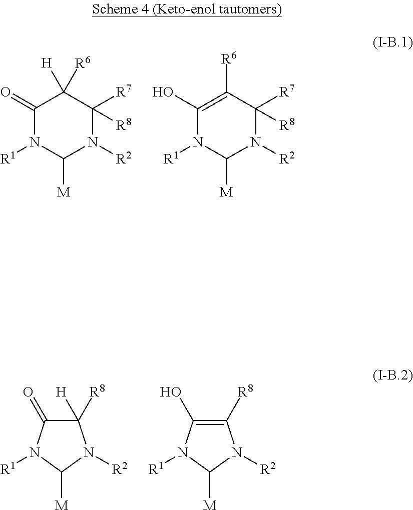

In Scheme 4, M, R¹, R², R⁶, R⁷ and R⁸ have the aforementioned meanings, in particular the meanings mentioned as preferred.

The enolizable compounds of the formula (I) obtained in step a) can be subjected to a further reaction with suitable electrophiles in step b1).

Accordingly, compounds of the general formula (I), wherein R³ and R⁴ together with the carbon atom to which they are bound are C═O, can be subjected e.g. to a further reaction with a compound R^{3a}—Z, where Z is a leaving group, in the presence of a suitable base. Preferably Z is selected from halides, tosylates, carboxylates, carbonates, esters, sulfonates and phosphates. Examples of Z are chlorine, bromine, iodine, methanesulfonate, trifluoromethanesulfonate and toluenesulfonate, with preference given to chlorine and bromine.

The base employed in step b1) is preferably a non-nucleophilic base, more preferably a hindered alkali amide base, e.g. lithium diisopropylamide, lithium bis(trimethylsilyl)amide, sodium bis(trimethylsilyl)amide, potassium bis(trimethylsilyl)amide.

The reaction in step b1) is usually carried out at a temperature in the range from −78° C. to ambient temperature, preferably in the range from −78 to 0° C.

Step a2)

In step a2) of the process according to the invention an isonitrile complex of the general formula (II) is reacted with an amine of the formula (V)

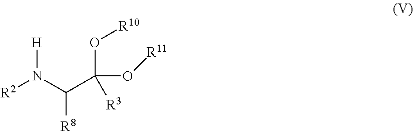

wherein
R², R³ and R⁸ have one of the meanings given above, especially one of the preferred ones; and
R¹⁰ and R¹¹ have one of the meanings given above; preferably R¹⁰ and R¹¹ are methyl or ethyl or R¹⁰ and R¹¹ together form an 1,2-ethylene or 1,3-propylene moiety, the carbon atoms of which may be unsubstituted or may all or in part be substituted by methyl groups.
to give an intermediate compound of the formula VI

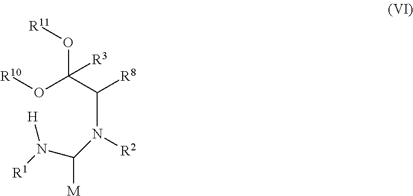

where
R¹, R², R³, R⁸, R¹⁰, R¹¹ and M have one of the meanings given above, especially one of the preferred ones.

The reaction is usually carried out in an inert organic solvent. Suitable solvents are halogenated hydrocarbons, such as dichloromethane or trichloromethane, and ethers, such as diisopropyl ether, tert.-butyl methyl ether, dioxane, anisole, tetrahydrofuran and dimethoxyethane. The reaction is usually carried out at temperatures of from 0° C. to 80° C., preferably from 10° C. to 40° C.

The reaction in step a2) can be monitored e.g. by the shift of the IR-streching frequencies of the isonitrile ligands. All of the yields were excellent.

According to a further embodiment, the process depicted in step a2) is used for the formation of compounds of the formula VI, where R¹ and R² have different meanings.

Preferably, in the compounds of the formula VI, R¹ is preferably selected from groups of the formulae IV.1 to IV.5, with particular preference given to the formulae IV.1 and IV.2. More preferably, R¹ is selected from C₁-C₆-alkyl, phenyl and phenyl which carries 1, 2 or 3 radicals independently selected from C₁-C₆-alkyl and chlorine. In particular preferably, R¹ is selected from tert.-butyl, 2,6-dimethylphenyl, 2,4,6-trimethylphenyl, 2,6-diisopropylphenyl and (2-chloro-6-methyl)phenyl.

Preferably, in the compounds of the formula VI, R² is selected from alkyl and cycloalkyl, in particular C₁-C₆-alkyl, phenyl-C₁-C₆-alkyl and C₅-C₁₅-cycloalkyl. In particular, R² is selected from methyl, ethyl, n-propyl, isopropyl, n-butyl, sec-butyl, tert-butyl, isobutyl, cyclopentyl, cyclohexyl, cyclododecyl and 1-adamantyl.

Preferably, in the compounds of the formula VI, R³ and R⁸ are independently of each other selected from hydrogen, C₁-C₆-alkyl and C₆-C₁₀-aryl.

Preferably, in the compounds of the formula VI, R¹⁰ and R¹¹ are, independently of each other, selected from C₁-C₄-alkyl.

Preferably, in the compounds of the formula VI, M is PdCl₂(CNR¹), PtCl₂(CNR¹), PdCl(CNR¹)₂, Au (CNR¹) and AuCl, where R¹ is selected from hydrogen, alkyl, cycloalkyl, heterocycloalkyl, aryl and hetaryl. In particular, M is AuCl.

Compounds of the formula VI are novel and thus form also part of the invention. They usually are air and moisture stable and can be stored at room temperature without de-composition.

Step b2)

In step b2), the intermediate compound of the formula (VI) is treated with an acid to obtain in situ the corresponding compound (VII) with a carbonyl functional group and subsequently intramolecular ring closure to obtain the compound of the formula I-E as depicted in scheme 5.

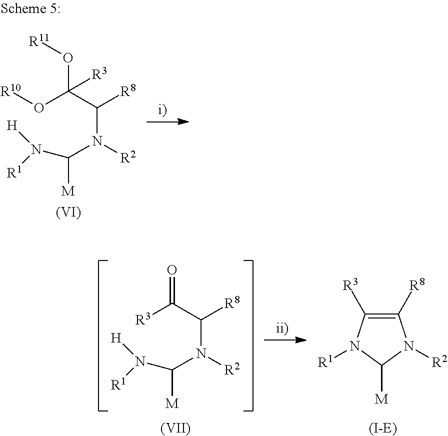

In scheme 5, R¹, R², R³, R⁸, R¹⁰, R¹¹ and M have one of the meanings given above, especially one of the preferred ones.

In step i) of scheme 5, compounds VI is treated with an acid to give the intermediate compound VII which reacts further to compound I-E in step ii). Suitable acids are inorganic or organic acids. Examples of suitable inorganic acids are mineral acids such as HCl or organic acids such as p-toluenesulfonic acid. Step i) of scheme 5 is usually carried out under hydrolytic conditions. Suitable solvents are those mentioned in step a1).

The reaction temperature is usually in the range from −10 to 100° C., preferably in the range from 0 to 50° C.

Compounds of the formula V can be obtained as depicted in scheme 6.

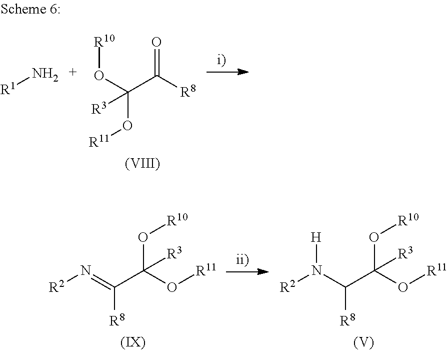

In Scheme 6, R¹, R², R^{3′} R⁸, R¹⁰ and R¹¹ have one of the meanings given above, in particular one of those being preferred.

In step i) of scheme 6, the amine R¹—NH₂ is treated with a compound of the formula (VIII) to give the imine of the formula (IX). The reaction is advantageously carried out in the presence of a dehydrating agent such as magnesium sulfate. The reaction is usually carried out in the presence of a solvent. Suitable solvents are halogenated aliphatic, alicyclic or aromatic hydrocarbons such as dichloromethane. In step ii) of scheme 6, the imine compound of the formula IX is reduced to yield the amine compound V. Suitable reducing agents are hydrides such as lithium aluminium hydride or sodium borohydride. The reaction is usually carried out in the presence of a solvent. Suitable solvents are C₁-C₄-alkanols, e.g. methanol or ethanol.

Step a3)

In the process according to the invention, the desired NHC complexes according to variant a3) having the formula I-F,

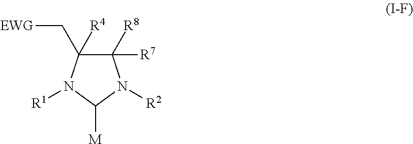

where R¹, R², R⁴, R⁷, R⁸, M and EWG have one of the meanings given above, preferably one of the preferred ones,
can be prepared by reacting the isonitrile complexes of the formula IIa with an amine of the formula IIIb or IIIc

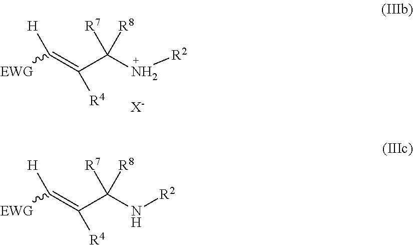

where
R², R⁴, R⁷ and R⁸ have one of the meanings given above;
X⁻ is an anion equivalent; and
EWG is C(O)R¹⁴, C(O)OR¹⁴, NO₂, S(O)R¹⁴ or S(O)₂R¹⁴, where R¹⁴ is hydrogen, alkyl, cycloalkyl or aryl.

The reaction is usually carried out in an organic solvent. Suitable organic solvents are aprotic solvents such as a haloalkanes, e.g dichloromethane.

The compounds of the general formula (I) according to the invention and/or obtained by the process of the invention can advantageously be employed in NHC metal complex catalyzed reactions. The compounds of the general formula (I) are preferably used as or in a catalyst employed in a C—C, C—O, C—N or C—H bond formation reaction. They are especially used in a C—C coupling reaction, selected from the Suzuki reaction, Heck reaction, Sonogashira reaction, Stille reaction, Hartwig-Buchwald reaction and Kumada reaction. Further, they are especially used in a reaction, selected from the hydrogenation, hydroformylation, hydrosilylation, Hartwig-Buchwald reaction and amide α-arylation. (With regard to C—C bond formation by cross-coupling, see: S. P. Nolan and O. Navarro in Comprehensive Organometallic Chemistry III, Vol. 11, 1^{st} ed. (Ed.: A. Canty), Elsevier, Oxford, 2007, Chapter 11.01, 1-38, and references therein; b) F. Glorius, Top. Organomet. Chem. 2007, 21, 1-20; c) E. A. B. Kantchev, C. J. O'Brien and M. J. Organ, Aldrichimica Acta 2006, 39, 97-111)

### Suzuki Cross-Coupling (Suzuki-Miyaura Cross-Coupling)

The palladium-catalyzed cross-coupling reaction between organoboron compounds and organic halides or triflates provides a powerful and general method for the formation of carbon-carbon bonds. For their fundamental work in the field of palladium-catalyzed cross couplings, the Nobel Prize in chemistry 2010 was awarded jointly to A. Suzuki, R. F. Heck and E.i Negishi. Information on the use of NHC metal complexes in the Suzuki reaction can be found in: a) W. A. Herrmann, C. P. Reisinger, M. Spiegler, J. Organomet. Chem. 1998, 557, 93; b) C. M. Zhang, J. K. Huang, M. L. Trudell, S. P. Nolan, J. Org. Chem. 1999, 64, 3804; c) A. Füirstner, A. Leitner, Synlett. 2001, 290; d) C. W. K. Gstottmayr, V. P. W. Böhm, E. Herdtweck, M. Grosche, W. A. Herrmann, Angew. Chem. Int. Ed. 2002, 41, 1363.

Preferably, the compounds of the general formula (I) are used in a reaction

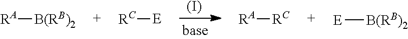

wherein
R^{A} is selected from in each case unsubstituted or substituted alkyl, alkenyl, alkinyl and aryl,
R^{B} is selected from alkyl, alkoxy and hydroxyl,
R^{c} is selected from in each case unsubstituted or substituted alkyl, alkenyl and aryl,
E is selected from Cl, Br, I, CF₃SO₃, (OR^{D})₂P(═O)O, wherein R^{D} is hydrogen, alkyl, cycloalkyl or aryl,
   the base is preferably selected from an alkali metal hydroxide, earth alkali metal hydroxide, alkali metal carbonate, earth alkali metal carbonate, thallium(I) hydroxide, thallium (I) alkanolate, alkali metal phosphate, alkali metal fluoride. Examples for suitable bases are NaOH, KOH, Na₂CO₃, K₂CO₃, Cs₂CO₃, Ba(OH)₂, K₃PO₄, TlOH, thallium(I) ethoxide, KF, CsF, (C₄H₉)₄NF, sodium ethoxide, potassium ethoxide and potassium tert-butoxide.

In particular, the compounds of the general formula (I) are used in the following reactions

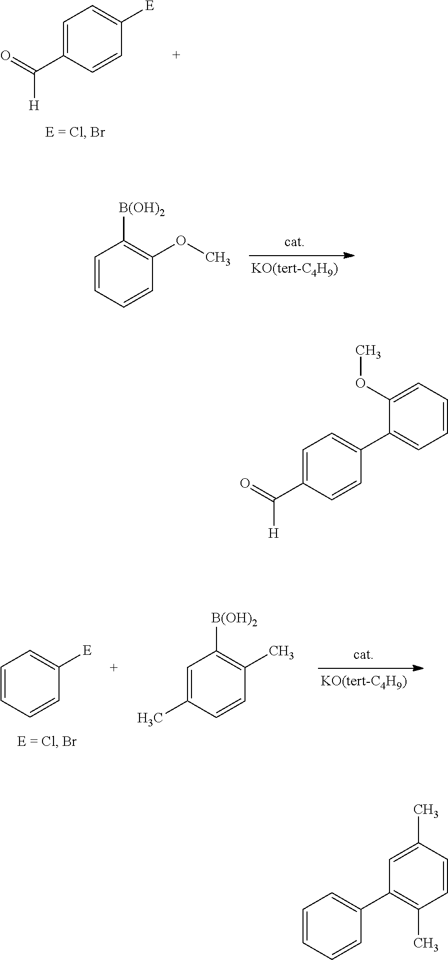

In these reactions, “cat.” refers to a catalyst of the formula (I).

### Sonogashira Reaction

The Sonogashira cross-coupling reaction is a palladium (and usually also copper) catalyzed coupling of terminal alkynes with aryl halides or vinyl halides to give enynes. Information on the use of NHC metal complexes in the Sonogashira reaction can be found in: a) S. Caddick, F. G. N. Cloke, G. K. B. Clentsmith, P. B. Hitchcock, D. McKerrecher, L. R. Titcomb, M. R. V. Williams, J. Organomet. Chem. 2001, 617; b) C. L. Yang, S. P. Nolan, Organometallics 2002, 21, 1020; c) L. Ray, S. Barman, M. M. Shaikh, P. Ghosh, Chem. Eur. J. 2008, 14, 6646.

Preferably, the compounds of the general formula (I) are used in a reaction

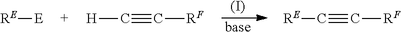

wherein
R^{E} is selected from in each case unsubstituted or substituted alkenyl, aryl and hetaryl,
R^{F} is selected from hydrogen, alkyl, alkenyl, aryl and Si(R^{G})₃,
R^{G} is selected from in each case unsubstituted or substituted alkyl, cycloalkyl and aryl,
E is selected from Cl, Br, I, CF₃SO₃,
   the base being usually an amine, alkanolate, earth alkali metal carbonate or alkalimetal carbonate. Preferably, the base is selected from secondary alkylamines, tertiary alkylamines, alkali metal alkanolates and alkali metal carbonates. The amine compound may be used in excess and serves also as solvent.

Preferred solvents for the Sonogashira reaction are CH₃CN, DMF (dimethylformamide) THF (tetrahydrofuran) or ethyl acetate.

Preferred base for the Sonogashira reaction are N(C₂H₅)₃, HN(C₂H₅)₂, N(iso-C₃H₇)₂(C₂H₅), KO(tert-C₄H₉), K₂CO₃ and Cs₂CO₃.

In particular, the compounds of the general formula (I) are used for the reaction of bromobenzene and 1-hexyne. No additional copper source was used.

Information on the use of NHC metal complexes in the Heck reaction can be found in: a) W. A. Herrmann, M. Elison, J. Fischer, C. Köcher, G. R. J. Artus, Angew. Chem. Int. Ed. 1995, 34, 2371; b) E. A. B. Kantchev, C. J. O'Brien, M. G. Organ, Angew. Chem. Int. Ed. 2007, 46, 2768; c) J. Ye, W. Chen, D. Wang, Dalton Trans. 2008, 30, 4015.

Information on the use of NHC metal complexes in the Stille reaction can be found in: G. A. Grasa, S. P. Nolan, Org. Lett. 2001, 3, 119.

Information on the use of NHC metal complexes in the Kumada reaction can be found in: V. P. W. Böhm, T. Weskamp, C. W. K. Gstöttmayr, W. A. Herrmann, Angew. Chem. Int. Ed. 2000, 39, 1602.

Information on the use of NHC metal complexes in the Hartwig-Buchwald-reaction can be found in: a) S. R. Stauffer, S. W. Lee, J. P. Stambuli, S. I. Hauck, J. F. Hartwig, Org. Lett. 2000, 2, 1423, b) J. Huang, G. Grasa, S. P. Nolan, Org. Lett. 1999, I, 1307.

Information on the use of NHC metal complexes in the α-arylation of amides can be found in: S. Lee, J. F. Hartwig, J. Org. Chem. 2001, 66, 3402.

Information on the use of NHC metal complexes in the hydrogenation can be found in: a) H. M. Lee, T. Jiang, E. D. Stevens, S. P. Nolan, Organometallics 2001, 20, 1255; b) L. D. Vazquez-Serrano, B. T. Owens, J. M. Buriak, Chem. Comm. 2002, 2518; c) D. Gnanamgari, E. L. O, Sauer, N. D. Schley, C. Butler, C. D. Incarvito, R. H. Crabtree, Organometallics 2009, 28, 321; d) H. Turkmen, T. Pape, F. Hahn, C. Ekkehardt; Eur. J. Inorg. Chem. 2008, 34, 5418.

Information on the use of NHC metal complexes in the hydroformylation can be found in: a) J. D. Scholten, J. Dupont, Organometallics 2008, 27, 4439.

Information on the use of NHC metal complexes in the hydrosilylation can be found in: b) W. A. Herrmann, L. J. Goossen, M. Spiegler, J. Organomet. Chem. 1997, 547, 357.

The following examples illustrate the invention without restricting it.

### EXAMPLES

### General Methods

All reagents and solvents were obtained from Acros, ABCR, Alfa Aesar, Sigma-Aldrich or VWR and were used without further purification unless otherwise noted. Deuterated solvents were purchased from Euriso-Top. Absolute solvents were dried by a MB SPS-800 with the aid of drying columns Preparation of air- and moisture-sensitive materials was carried out in flame dried flasks under an atmosphere of nitrogen using Schlenk-techniques. Cross coupling reactions were performed in technical grade solvents. Thin layer chromatography (TLC) was performed using Polygram® precoated plastic sheets SIL G/UV₂₅₄ (SiO₂, 0.20 mm thickness) from Macherey-Nagel. Column chromatography was performed using silica gel (40.0-63.0 nm particle size) from Macherey-Nagel. NMR spectra were recorded on Bruker Avance 500, Bruker Avance 300 and Bruker ARX-250 spectrometers. Chemical shifts (in ppm) were referenced to residual solvent protons. Signal multiplicity was determined as s (singlet), d (doublet), t (triplet), q (quartet) or m (multiplet). ¹³C assignment was achieved via DEPT90 and DEPT135 or HSQC-me spectra. MS spectra were recorded on a Vakuum Generators ZAB-2F, Finnigan MAT TSQ 700 or JEOL JMS-700 spectrometer. GC spectra were recorded on HP Agilent 5890 Series II Plus with FID analyser. GC-MS spectra were recorded on an Agilent 5890 Series II Plus with a HP 5972 mass analysator. IR spectra (in cm⁻¹) were recorded on a Bruker Vector 22 FT-IR. Crystal structure analysis was accomplished on Bruker Smart CCD or Bruker APEX diffractometers. Elemental analysis was performed on an Elementar Vario EL. ReactIR® studies were performed on a ReactIR® Mettler Toledo IC10.

### Syntheses

### I. Preparation of Starting Materials

### I.0 General Synthesis of Formamides

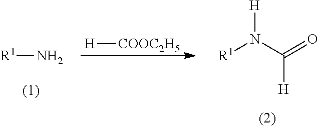

R¹ is as defined above.

The amine (1) was dissolved in ethyl formate (15 mL) and was heated in an autoclave at 200° C. for 12 h and at 250° C. for another 5 h. The precipitate was filtered off and washed with n-pentane. Recrystallisation from acetone/petrol ether (1/5) yielded the formamide compound (2) as colourless crystal.

### I.1 Synthesis of Isonitrile Ligands

### I.1.a General Procedure for Preparing Isonitrile Compounds

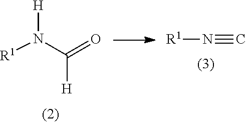

R¹ is as defined above.

The formamide (2) was dissolved in absolute dichloromethane. The solution was cooled to −60° C. and POCl₃ was added dropwise over a period of 5 min. The suspension was stirred for 20 min and triethylamine was added dropwise over 10 min. The resulting yellow suspension was stirred overnight. During this time, the cooling bath was allowed to warm up to room temperature. Afterwards, the suspension was poured onto ice and warmed up to room temperature. Dichloromethane was added and the layers were separated. The organic layer was washed 3 times with a saturated aqueous solution of Na—HCO₃. The organic phase was dried with Na₂SO₄ and the solvent was removed under reduced pressure. The crude product was purified by destillation or column chromatography (SiO₂).

### Example I

### 2,4,6-trimethylphenyl isonitrile

2,4,6-trimethylaniline (10 g, 73.9 mmol) was dissolved in ethylformiate (15 ml). The mixture was heated in an autoclave at 200° C. for 12 h. After this, the solid precipitate was filtered off and washed with pentane. Recrystallization from acetone/petrolether (1/3) yielded N-(2,4,6-trimethylphenyl)formamide as colourless crystals in almost quantitative yield (11.43 g, 96%). All analytical data are in good agreement with the previously reported ones (G. Vougioukalakis, J. Am. Chem. Soc. 2008, 130, 2234-2245).

The formamide (1 g, 6.13 mmol) was dissolved in absolute dichloromethane (DCM) (30 ml). The solution was cooled to −60° C. in an ethanol/liquid nitrogen bath and POCl₃ (1.67 ml, 18.3 mmol) was added drop wise over a period of 5 min. The suspension was stirred for 20 min and triethylamine (5.57 g, 55.0 mmol) was added drop wise over 10 min. The resulting yellow suspension was stirred over night. During this time, the cooling bath was allowed to warm up to room temperature. Afterwards, the suspension was poured onto ice and warmed up to room temperature. DCM (30 ml) was added and the layers were separated. The organic layer was washed with a saturated solution of Na—HCO₃ (3×10 ml). The organic phase was dried with NaSO₄ and the solvent was removed under reduced pressure. The crude product was purified by sublimation (50-55° C., 6.5×10⁻² mbar) to yield the title compound as colourless crystals (637 mg, 72%).

¹H NMR (300 MHz, CD₂Cl₂): δ=2.23 (s, 3H, —CH₃), 2.31 (s, 6H, —CH₃), 6.95 (s, 2H, ArH); ¹³C NMR (75 MHz, CD₂Cl₂): δ=17.19, 19.53, 127.01, 133.16, 137.55 (no further signals observed). All analytical data are in good agreement with previously reported ones (G. Vougioukalakis, J. Am. Chem. Soc. 2008, 130, 2234-2245).

### Example II

### 2,6-diisopropylphenyl isonitrile

2,6-diisopropylaniline (purity: 92%, 10 g, 51.9 mmol) was dissolved in ethylformiate (15 ml). The mixture was heated in an autoclave first at 200° C. for 12 h after this at 250° C. for 5 h. The solid precipitate was filtered off and washed with pentane. Recrystallization from acetone/petrol ether (1/5) yielded N-(2,6-diisopropylphenyl)formamide as colourless crystals in almost quantitative yield (9.48 g, 46.2 mmol, 89%). The formamide (1 g, 4.87 mmol) was dissolved in absolute DCM (10 ml). The solution was cooled to −60° C. in an ethanol/liquid nitrogen bath and POCl₃ (1.33 ml, 14.6 mmol) was added drop wise over a period of 5 min. The suspension was stirred for 20 min and triethylamine (4.43 g, 43.8 mmol) was added drop wise over 10 min. The resulting yellow suspension was stirred over night. During this time, the cooling bath was allowed to warm up to room temperature. Afterwards, the suspension was poured onto ice and warmed up to room temperature. DCM (30 ml) was added and the layers were separated. The organic layer was washed with a saturated solution of NaHCO₃ (3×10 ml). The organic phase was dried with Na₂SO₄ and the solvent was removed under reduced pressure. The crude product was purified by distillation (72-80° C., 4.5×10⁻² mbar) to yield the title compound as colourless oil (720 mg, 3.84 mmol, 79%).

¹H NMR (300 MHz, CD₂Cl₂): δ=1.22 (d, 12H, J=7.0 Hz, —CH₃), 3.32 (m, 2H, J=6.9 Hz, —CH—), 7.14 (d, 2H, J=7.8 Hz, ArH), 7.29 (t, 1H, J=7.7 Hz, ArH); ¹³C NMR (75 MHz, CD₂Cl₂): δ=23.12, 30.62, 123.94, 130.05, 145.72 (no further signals observed). All analytical data are in good agreement with previously reported ones (U. J. Kilgore, F. Basuli, J. C. Huffman, D. J. Mindiola, Inorg. Chem. 2006, 45, 487-489).

### I.2 Synthesis of Isonitrile-Pd Complexes

### General Procedure:

[Pd(CH₃CN)₂Cl₂] was dissolved in toluene and two equivalents of the isonitrile was added. The mixture was stirred 12 h at room temperature. The precipitate was filtered off, washed with cold pentane and dried under reduced pressure to afford the title compounds.

### Example III

### cis-[PdCl₂(2,6-dimethylphenyl isonitrile)₂]

[Pd(CH₃CN)₂Cl₂] (200 mg, 780 μmol) was dissolved in toluene (8 ml) and 2,6-dimethylphenyl isonitrile from example II (212 mg, 1.60 mmol) was added. The mixture was stirred 12 h at ambient temperature. The precipitate was filtered off, washed with cold pentane (3×10 ml) and dried under reduced pressure to yield the title compound as white solid (333 mg, 757 μmol, 97%).

IR (KBr): ν=2363, 2208, 1632, 1473, 1384, 1170, 771, 717, 576, 499, 453; HRMS (FAB⁺) C₁₈H₁₈N₂ClPd [M−Cl⁻]⁺: calc. 403.0193. found: 403.0138.

The compounds of the examples IV, V, VI and VII listed below were prepared in an analogous manner to example III.

### Example IV

### cis-[Pd(2,4,6-trimethylphenyl isonitrile)₂Cl₂]

The title compound was obtained as white solid (yield: 89%).

IR (KBr): ν=2919, 2210, 1604, 1471, 1382, 1308, 1035, 853, 712, 600, 568, 502, 473; HR-MS (FAB⁺): calc.: C₂₀H₂₂PdClN₃[M−Cl⁻]⁺=431.0506. found: 431.0486

### Example V

### cis-[PdCl₂(2,6-diisopropylphenyl isonitrile)₂]

The title compound was obtained as a yellow solid (yield: 83%).

¹H NMR (300 MHz, CD₂Cl₂): δ=1.21 (d, 12H, J=6.9 Hz, —CH₃), 3.26 (m, 2H, —CH—), 7.17 (d, 2H, J=7.8 Hz, ArH), 7.40 (t, 1H, J=7.9 Hz, ArH); ¹³C NMR (75 MHz, CD₂Cl₂): δ=23.24 (q, 8C), 30.78 (d, 4C), 124.77 (d, 6C), 132.52 (s, 4C), 147.24 (s, 2C); IR (KBr): ν=2966. 2927. 2873, 2209, 1635, 1585, 1475, 1458, 1433, 1388, 1366, 1356, 1259, 1183, 1062, 800, 751, 510, 477 cm⁻¹; HR-MS (FAB⁺): m/z=515.1140, calcd. for C₂₆H₃₄ClN₂Pd [M−Cl⁻]⁺: 515.1145, m/z=480.1760, calcd. for C₂₆H₃₄N₂Pd [M−2Cl⁻]⁺: 480.1757.

### Example VI

### cis-[PdCl₂(2-chloro-6-methylphenyl isonitrile)₂]

The title compound was obtained as a white solid (yield: 95%).

¹H NMR (300 MHz, CDCl₃): δ=2.45 (s; 3H, —CH₃), 7.09-7.26 (m; 3H, ArH); IR (KBr): ν=2206, 1632, 1461, 1177, 873, 782, 711, 569 ¹³C NMR (75 MHz, CDCl₃): δ=19.34, 77.43, 127.96, 129.19, 129.59, 131.78, 132.09, 139.68; HRMS (FAB⁺) C₁₆H₁₂N₂Cl₃Pd [M−Cl⁻]⁺: calc. 442.9101. found: 442.9069

### Example VII

### cis-[PdCl₂(tert-butyl)₂]

The title compound was obtained as a white solid (yield 92%). The analytical data are in good agreement with the previously reported ones S. Otsuka, Y. Tatsuno, K. Ataka, J. Am. Chem. Soc 1971, 93, 6705-6706.

### I.3 Synthesis of Isonitrile-Au(I) Complexes

### General Procedure:

One equivalent of [AuCl(tetrahydrothiophene)] was dissolved in dichloromethane (DCM) and 1 equiv. isonitrile was added at room temperature. The mixture was stirred for 15 min. The solvent was removed under reduced pressure. The crystalline crude product was used without further purification.

### Example VIII

### [AuCl(2,4,6-trimethylphenyl isonitrile)]

[AuCl(tetrahydrothiophene)] (500 mg, 1.56 mmol) was dissolved in DCM (10 ml) and 2,4,6-trimethylphenyl isonitrile from example I (233 mg, 1.56 mmol) was added. The mixture was stirred 12 h at ambient temperature. After this, the solvent was removed and the resulting white precipitate was washed with pentane (3×10 ml) to yield the complex as white solid (583 mg, 1.54 mmol, 99%).

¹H NMR (300 MHz, CD₂Cl₂): δ=2.28 (s, 6H, CH₃), 2.34 (s, 3H, CH₃), 6.95 (s, 2H, ArH); ¹³C NMR (75 MHz, CD₂Cl₂): δ=18.76, 21.59, 129.50, 136.42, 142.30; IR (KBr): ν=2917, 2207 (—NC), 1602, 1474, 1387, 1308, 1201, 1040, 857, 753, 713, 567, 493; HR-MS (FAB⁺): calc.: C₁₀H₁₂AuClN [M+H]⁺=378.0324. found: 378.0332.

The compounds of the examples IX and X listed below were prepared in an analogous manner to example VIII.

### Example IX

### [AuCl(2,6-diisopropylphenyl isonitrile)]

The title compound was obtained in a yield of 99%.

¹H NMR (300 MHz, CD₂Cl₂): δ=1.24 (d, 12H, J=6.9 Hz, —CH₃), 3.2 (m, 2H, J=6.8 Hz, —CH—), 7.22 (d, 2H, J=7.9 Hz, ArH), 7.45 (t, 1H, J=7.8 Hz, ArH); ¹³C NMR (75 MHz, CD₂Cl₂): δ=22.75 (q, 4C), 30.43 (d, 2C), 124.42 (d, 3C), 132.06 (s, 2C), 146.87 (s); IR (KBr): ν=2946, 2867, 2208 (—NC), 1632, 1462, 1385, 1364, 1261, 1185, 1109, 1062, 936, 799, 750, 534; HR-MS (FAB⁺): calc.: C₁₃H₁₇AuN [M−Cl⁻]⁺=384.1027. found: 384.1030

### Example X

### [AuCl(tert-butyl isonitrile)]

The title compound was obtained in a yield of 99%. All analytical data are in good agreement with the previously reported ones R. Heathcote, J. A. S. Howell, N. Jennings, D. Cartlidge, L. Cobden, C. Coles, M. Hursthouse, Dalton Trans. 2007, 13, 1309-1315.

### I.4 Synthesis of Isonitrile-Pt(II) Complexes

### General Procedure:

[Pt(CH₃CN)₂Cl₂] was dissolved in CHCl₃ and an appropriate isonitrile was added. The mixture was stirred 12 h at reflux. The solvent was removed and the resulting colourless solid was washed with pentane affording the isonitrile-Pt(II) complexes.

### Example XI

### cis-[PtCl₂(2,6-diisopropylphenyl isonitrile)₂]

[Pt(CH₃CN)₂Cl₂] (272 mg, 780 μmol) was dissolved in CHCl₃ (15 ml) and 2,6-dimethylphenylisonitrile (212 mg, 1.60 mmol) was added. The mixture was stirred 12 h at reflux. The solvent was removed and the resulting colourless solid was washed with pentane (3×10 ml) yielding the title compound (489 mg, 749 μmol, 96%). ¹H NMR (500 MHz, CD₂Cl₂): δ=1.25 (d, 12H, J=6.9 Hz, —CH₃), 3.29 (m, 2H, J=6.9 Hz, —CH—), 7.22 (d, 2H, J=7.8 Hz, ArH), 7.42 (t, 1H, J=7.8 Hz, ArH), ¹³C-NMR (125 MHz, CD₂Cl₂): δ=22.89 (q, 8C), 30.38 (d, 4C), 124.31 (d, 6C), 131.72 (s, 4C), 146.67 (s, 2C); IR (KBr): ν=2966, 2929, 2869, 2219, 2189, 1475, 1465, 1457, 1436, 1386, 1365, 1184, 1062, 938, 804, 797, 747, 736, 491 cm⁻¹; HR-MS (FAB⁺): m/z=604.2060, calcd. for C₂₆H₃₄ClN₂Pt [M−Cl⁻]⁺: 604.2058, m/z=569.2372, calcd. for C₂₆H₃₄N₂Pt [M−2Cl⁻]⁺: 569.2370.

I.5 2-(Chloroethyl)Ammonium Chlorides of the General Formula III (Y═Cl)

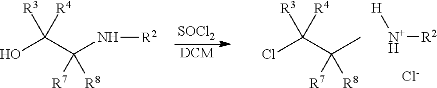

R², R³, R⁴, R⁷ and R⁸ have the aforementioned meanings, DCM is dichloromethane.

The synthesis of the 2-(chloroethyl)ammonium chlorides has been accomplished according to literature procedures, e.g. A. Habtemariam, B. Watchman, B. S. Potter, R. Palmer, S. Parsons, A. Parkin, P. J. Sadler, J. Chem. Soc., Dalton Trans. 2001, 8, 1306-1318 starting from commercially available amino alcohols.

### I.6 Synthesis of Amino Alcohols

### Example XII

### 2-(adamantan-1-ylamino)ethanol

1-Adamantylamine (1 g, 6.05 mmol) and 2-iodoethanol (1.20 g, 7.00 mmol) were dissolved in benzonitrile (2 ml). The mixture was heated at 120° C. for 12 h. After this, the precipitate was filtered off and carefully washed with petrolether (3×20 ml). The white solid was dissolved in DCM (30 ml) and washed with a saturated solution of Na₂CO₃ (3×50 ml). The organic layer was separated, dried with Na₂SO₄ and the solvent was removed under reduced pressure to yield the title compound as colourless oil (950 mg, 4.86 mmol, 80%).

¹H NMR (250 MHz, CD₂Cl₂): δ=1.55 (m, 12H, —CH₂—), 1.99 (bs, 5H, —CH— and over-lapping OH, NH), 2.65 (t, 2H, J=5 Hz, —CH₂—), 3.44 (t, 2H, J=5.1 Hz, —CH₂—); all spectroscopic data are in good agreement with previously reported ones, e.g. P. E. Aldrich, E. C. Herrmann, W. E. Meier, M. Paulshock, W. W. Prichard, J. A. Snyder, J. C. Watts, J. Med. Chem. 1971, 14, 535-543.

I.7 Preparation of n-(2,2-dimethoxyethyl)amines of the General Formula V

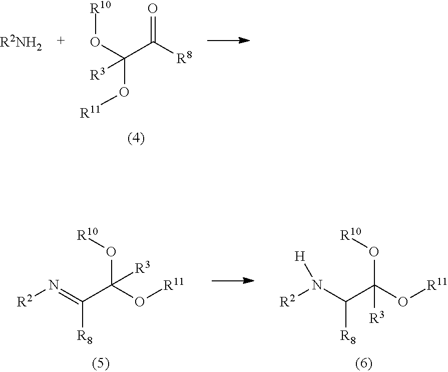

R², R³, R⁸, R¹⁰ and R¹¹ have the aforementioned meanings.

In a typical protocol, one equivalent of the amine R²NH₂ was dissolved in dichloromethane and MgSO₄ and 1.5 equivalents of compound (4) were added. The mixture was stirred for 12 h at room temperature. Afterwards, the MgSO₄ was filtered off and the solvent evaporated. The analytically pure imines were used without further purification. The obtained imines were dissolved in dry methanol and 2 equiv. of NaBH₄ were added at 0° C. After removal of the ice bath, the mixture was stirred for another 2 h. The reaction was quenched with water, diluted with dichloromethane and the organic phase was washed with saturated NH₄Cl solution and brine. The crude product was purified by distillation or column chromatography (SiO₂) to give the title compound

### Example XIII

### N-(2,2-Dimethoxyethyl)-2,4,6-trimethylaniline

The title compound was prepared according to the general procedure using 2.50 g (18.5 mmol) of mesitylamine, 3.2 g (27.3 mmol) of 2,2-dimethoxyacetaldehyde (60% in water) and 3.00 g of MgSO₄ in 100 ml of dichloromethane. After filtration of the solvent, N-(2,2-dimethoxyethylidene)-2,4,6-trimethylaniline was afforded as a yellowish solid; yield: 4.05 g (18.3 mmol, 99%). ¹H-NMR (300 MHz, CDCl₃): δ=2.07 (s, 6H, CH₃), 2.24 (s, 3H, CH₃), 3.31 (s, 6H, OCH₃), 4.87 (d, J=4.5 Hz, 1H, CH(OMe)₂), 6.83 (s, 2H, ArH), 7.42 (d, J=4.5 Hz, 1H, CH═N); ¹³C-NMR (75 MHz, CDCl₃): δ=18.31 (q, 2C), 20.79 (q), 54.32 (q, 2C), 103.61 (d), 125.82 (d, 2C), 128.87 (s, 2C), 133.52 (s), 147.53 (s), 163.5 (d); IR (KBr): ν=3431, 2999, 2955, 2914, 2836, 1667, 1480, 1455, 1442, 1375, 1308, 1215, 1194, 1147, 1098, 1065, 1004, 983, 849, 782 cm⁻¹; HR-MS (EI⁺): m/z=221.1388, calcd. for C₁₃H₁₉O₂N [M]⁺: 221.1416.

N-(2,2-dimethoxyethylidene)-2,4,6-trimethylaniline (2.40 g, 10.9 mmol) was dissolved in 50 ml of dry methanol under an atmosphere of nitrogen and 533 mg of NaBH₄ (14.1 mmol) was added. After workup the crude product was purified by destillation (120° C., 6×10⁻² mbar); yield: 2.12 g (mmol, 87.1%); ¹H-NMR (300 MHz, CDCl₃): δ=2.27 (s, 3H, CH₃), 2.31 (s, 6H, CH₃), 3.12 (d, J=6.4 Hz, 1H, CH(OMe)₂), 3.26 (bs, NH), 3.44 (s, 6H, OCH₃), 4.51 (t, J=6.4 Hz, 2H, CH₂), 6.86 (s, 2H, ArH); ¹³C-NMR (75 MHz, CDCl₃): δ=18.27 (q, 2C), 20.57 (q), 49.73 (t), 53.74 (q, 2C), 103.46 (t), 129.43 (d, 2C), 131.38 (q, 2C), 131.38 (d), 143.05 (s); IR (KBr): ν=3379, 2936, 2856, 2832, 1486, 1447, 1376, 1306, 1236, 1194, 1157, 1131, 1074, 1034, 977, 924, 854, 739, 564 cm⁻¹; HR-MS (EI⁺): m/z=223.1573, calcd. for C₁₃H₂₁O₂N [M]⁺: 223.1572.

I.8 Synthesis of Compounds of Formula IIIb with EWG being CO(O)Ch₃

### Example XIV

### (E)-Methyl 4-(cyclododecylamino)but-2-enoate

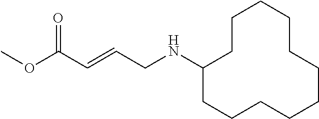

Under an atmosphere of nitrogen (E)-methyl 4-bromobut-2-enoate (5.70 g, 31.8 mmol), cyclododecanamine (6.41 g, 35.0 mmol) and Cs₂CO₃ were suspended in absolute tetra-hydrofuran (THF) (150.0 ml). The mixture was heated at reflux for 12 h and NH₄Cl (saturated solution, 100 ml) was added. The mixture was extracted with DCM (three times, 50.0 ml). The organic layer was dried with Na₂SO₄ and the solvent was removed under reduced pressure. The crude product was purified by column chromatography using silica and petrol ether/ethyl acetate (4:1) to yield the title compound as yellow solid (5.54 g, 19.7 mmol, 62%). ¹H NMR (300 MHz, CD₂Cl₂) δ=1.07-1.54 (m, 23H, —CH₂—, —NH—), 2.67 (m, 1H, —CH—), 3.44 (dd, J=5.4, 2.2 Hz, 2H, —CH₂—), 3.75 (s, 3H, —CH₃), 6.02 (dd, J=15.7, 2.1 Hz, 1H, ═CH—), 7.01 (dq, J=15.7, 5.2 Hz, 1H, ═CH—).

### II. Synthesis of NHC-(Transition Metal) Complexes of the Formula I

### II.1 Synthesis of NHC-(Transition Metal) Complexes of the Formula I-a.2.1

### II.1.1 Synthesis of NHC-Pd(II) Complexes

### General Procedure for the Synthesis of NHC-Pd(II) Complexes

In a typical protocol the cis-(isonitrile)-Pd(II) complex (321 μmol) and the 2-(chloroethyl)ammonium chloride (350 μmol) were suspended in absolute THF. triethyl amine (0.5 ml, 6.81 mmol) was added. The mixture was stirred for 12 h at ambient temperature. After this all the volatiles were removed under reduced pressure and the solid was dissolved in DCM (20 ml). The solution was extracted with a saturated solution of NH₄Cl (20 ml). The organic layer was dried with Na₂SO₄ and the solvent was removed under reduced pressure. The crude product was dissolved in a minimum of DCM and covered with a layer diethyl ether, inducing crystallization of the NHC-Pd(II) complexes as colourless crystals. The crystals were filtered off, washed with diethyl ether and dried under reduced pressure. All the complexes of examples 1 to 15 listed below in table I are air and moisture stable compounds and can be stored at ambient temperature without decomposition.

TABLE I
(I-A.2.1) with M being PdCl₂(CNR¹)

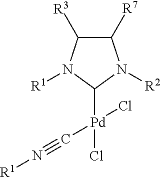

| | | | | | yield/material |
|---|---|---|---|---|---|
| Ex. | R¹ | R³ | R² | R⁷ | property |
| 1 | 2,6-dimethylphenyl | H | methyl | H | 85%, colourless |
| | | | | | crystals |
| 2 | 2,6-dimethylphenyl | H | isopropyl | H | 89%, colourless |
| | | | | | solid, |
| 3 | 2,6-dimethylphenyl | H | cyclohexyl | H | 78%, colourless |
| | | | | | solid |
| 4 | 2,6-dimethylphenyl | phenyl | cyclohexyl | H | 49%, yellow solid |
| 5 | 2,4,6-trimethylphenyl | H | isopropyl | H | 96%, colourless |
| | | | | | solid |
| 6 | 2,6- | H | isopropyl | H | 83%, colourless |
| | diisopropylphenyl | | | | solid |
| 7 | 2,6- | H | cyclohexyl | H | 95%, colourless |
| | diisopropylphenyl | | | | solid |
| 8 | 2,6- | H | 1-adamantyl | H | 95%, colourless |
| | diisopropylphenyl | | | | solid |
| 9 | 2,6- | phenyl | cyclohexyl | H | 60%, colourless |
| | diisopropylphenyl | | | | solid |
| 10 | tert-butyl | H | methyl | H | 72%, colourless |
| | | | | | solid |
| 11 | tert-butyl | H | isopropyl | H | 69%, colourless |
| | | | | | solid |
| 12 | tert-butyl | H | cyclohexyl | H | 52%, colourless |
| | | | | | solid |
| 13 | 2-methyl- | H | methyl | H | 58%, dark crystals |
| | 6-chlorophenyl | | | | |
| 14 | 2,6-dimethylphenyl | H | —CH═CH— | | 58%, colourless |
| | | | CH═CH— | | olid |
| 15 | 2,6- | H | cyclopenta- | H | 42%, colourless |
| | diisopropylphenyl | | decyl | | solid |

The physicochemical data of the complexes of the examples 1 to 14 are listed below:

### Example 1

¹H NMR (300 MHz, DMSO): δ=2.06 (s; 3H, —CH₃), 2.30 (s; 6H, —CH₃), 2.44 (s; 3H, —CH₃), 3.52 (s; 3H, —CH₃), 3.98 (m; 4H, —CH₂—), 7.25 (m; 6H, ArH); ¹³C NMR (75 MHz, DMSO): δ=17.35, 17.93, 18.68, 37.20, 51.05, 51.20, 128.31, 128.42, 128.93, 129.06, 130.68, 135.33, 135.62, 136.77, 137.71, 182.03; IR (KBr): ν=3443, 2951, 2919, 2195, 1631, 1541, 1491, 1473, 1411, 1383, 1317, 1276, 1116, 780, 734, 619; HRMS (FAB⁺) C₁₉H₂₅N₃ClPd [M−Cl⁻]⁺: calc. 460.0772. found: 460.0763.

### Example 2

¹H NMR (600 MHz, CD₂Cl₂): δ=1.34 (d, 3H, J=6.8 Hz, —CH₃), 1.39 (d, 3H, J=6.7 Hz, —CH₃), 2.01 (s, 3H, —CH₃), 2.27 (s, 6H, —CH₃), 2.47 (s, 3H, —CH₃), 3.82-3.91 (m, 4 H, —CH₂—), 5.40 (m, 1H, J=6.9, 6.3 Hz, —CH—), 6.94 (dd, 1H, J=7.3, 1.9 Hz, ArH), 7.1 (d, 2H, J=7.6 Hz, ArH), 7.17-7.27 (m, 3H, ArH); ¹³C NMR (125 MHz, CD₂Cl₂): δ=18.11, 18.92, 19.40, 20.13, 20.93, 43.61, 51.02, 52.25, 128.65, 128.70, 129.62, 130.01, 130.70, 135.64, 136.19, 137.30, 138.70, 184.22; ¹⁵N NMR (600 MHz, CD₂Cl₂, urea): δ=131.78, 148.43, 171.32; IR (KBr): ν=2970, 2925, 2358, 2195 (—NC), 1633, 1509, 1467, 1368, 1314, 1271, 1192, 1127, 1102, 1056, 935, 782, 623, 602, 569; HR-MS (FAB⁺): calc.: C₂₃H₂₉ClN₃Pd [M−Cl⁻]⁺=488.1085. found: 488.1104.

### Example 3

¹H NMR (300 MHz, CD₂Cl₂): δ=0.98-1.86 (m, 10H, —CH₂—), 1.98 (s, 3H, —CH₃), 2.23 (s, 6H, —CH₃), 2.44 (s, 3H, —CH₃), 3.81 (m, 4H, —CH₂—), 4.87 (tt, 1H, J=10.7, 3.8 Hz, —CH—) 6.9 (dd, 1H, J=6.6 Hz, ArH), 7.07 (d, 2H, J=8.4 Hz, ArH), 7.12 (m, 3H, ArH); ¹³C NMR (75 MHz, CD₂Cl₂): δ=18.49, 19.23, 19.80, 25.91, 26.04, 26.12, 31.18, 32.07, 45.24, 51.33, 60.10, 129.02, 129.06, 129.99, 130.38, 131.05, 136.04, 136.57, 139.11, 184.56; IR (KBr): ν=2931, 2855, 2193 (—NC), 1632, 1510, 1454, 1380, 1333, 1300, 1272, 1170, 1100, 1032, 990, 894, 780, 623, 570; HR-MS (FAB⁺): calc.: C₂₆H₃₃ClN₃Pd [M−Cl⁻]⁺=530.1398. found: 530.1397.

### Example 4

(Mixture of diastereoisomers) ¹H NMR (300 MHz, CD₂Cl₂): δ=1.05-1.81 (m, 10H, —CH₂), 1.92 (s, —CH₃), 2.06 (s, —CH₃), 2.17 (s, —CH₃), 2.39 (s, —CH₃), 2.55 (s, CH₃), 4.03 (dd, J=11.3, 5.6 Hz, —CH₂), 4.15 (dd, J=11.3, 6.0 Hz, —CH₂), 4.33 (t, J=11.3 Hz, —CH₂), 4.88 (dd, J=11.8, 5.6 Hz, —CH—), 5.03 (dd, J=11.6, 6.0 Hz, —CH—), 5.16 (m, 1H, —CH—), 6.73 (m, 1H, ArH), 6.96-7.32 (m, 10H, ArH); ¹³C NMR (75 MHz, CD₂Cl₂): δ=18.27, 18.51, 18.81, 19.08, 19.71, 19.87, 25.10, 25.44, 25.48, 30.24, 30.75, 31.38, 31.77, 50.75, 52.29, 59.58, 59.99, 66.51, 66.56, 77.44, 127.98, 128.01, 128.30, 128.49, 129.07, 129.20, 129.27, 129.57, 129.68, 129.90, 130.36, 130.45, 183.36, 183.98; IR (KBr): ν=3031, 2932, 2855, 2193, 1631, 1509, 1474, 1453, 1416, 1382, 1296, 1250, 1210, 1168, 1101, 1034, 998, 780, 701; HR-MS (FAB⁺): calc.: C₃₂H₃₇ClN₃Pd [M−Cl⁻]⁺=604.1711. found: 604.1722

### Example 5

¹H NMR (300 MHz, CD₂Cl₂): δ=1.32 (d, 3H, J=6.9 Hz, —CH₃), 1.37 (d, 3H, J=6.6 Hz, —CH₃), 1.95 (s, 3H, —CH₃), 2.22 (s, 6H, —CH₃), 2.25 (s, 3H, —CH₃), 2.26 (s, 3H, —CH₃), 2.41 (s, 3H, —CH₃), 3.71-3.92 (m, 4H, —CH₂—), 5.38 (m, 1H, J=6.8 Hz, —CH—), 6.73 (s, 1H, ArH), 6.90 (s, 2H, ArH), 7.00 (s, 1H, ArH); ¹³C NMR (75 MHz, CD₂Cl₂): δ=18.37, 19.14, 19.67, 20.50, 21.27, 21.58, 21.88, 43.86, 51.42, 52.53, 58.95, 129.69, 129.75, 131.02, 135.09, 135.56, 136.25, 138.54, 139.96, 141.77, 184.74; IR (KBr): ν=2971, 2193, 1608, 1510, 1458, 1382, 1314, 1271, 1197, 1105, 1058, 855, 713, 625, 599, 573, 460, 428; HR-MS (FAB): calc.: C₂₅H₃₃ClN₃Pd [M−Cl⁻]⁺=516.1398. found: 516.1391

### Example 6

¹H NMR (300 MHz, CD₂Cl₂): δ=1.05 (d, 3H, J=3.3 Hz, —CH₃), 1.07 (d, 3H, J=3.4 Hz, —CH₃), 1.13 (d, 3H, J=7.0 Hz, —CH₃), 1.19 (d, 6H, J=6.8 Hz, —CH₃), 1.21 (d, 6H, J=6.8 Hz, —CH₃), 2.88 (m, 1H, J=6.8 Hz, —CH—), 3.24 (m, 3H, —CH—), 3.71-4.01 (m, 4 H, —CH₂—), 5.47 (dm, 1H, J=7.0, 6.6 Hz, —CH—), 7.18, 7.17 (dd, 1H, J=7.7, 1.4 Hz, ArH), 7.19 (d, 2H, J=7.7 Hz, ArH), 7.29 (dd, 1H, J=7.8, 1.7 Hz, ArH), 7.40 (m, 2H, ArH); ¹³C NMR (75 MHz, CD₂Cl₂): δ=20.65, 21.17, 23.49, 23.63, 23.87, 24.51, 27.22, 27.26, 29.29, 29.54, 30.36, 43.67, 53.03, 124.73, 125.01, 126.19, 130.76, 131.80, 134.57, 146.85, 146.96, 149.31, 185.97; IR (KBr): ν=2965, 2930, 2870, 2185, 1497, 1459, 1433, 1386, 1366, 1348, 1331, 1315, 1267, 1184, 1113, 1058, 806, 751, 628; HR-MS (FAB): calc.: C₃₁H₄₅ClN₃Pd [M−Cl⁻]⁺=600.2337. found: 600.2335

### Example 7

¹H NMR (500 MHz, CD₂Cl₂): δ=1.03 (d, 3H, J=6.9 Hz, —CH₃), 1.04 (d, 3H, J=6.9 Hz, —CH₃), 1.11 (d, 3H, J=6.9 Hz, —CH₃), 1.17 (d, 6H, J=6.8 Hz, —CH₃), 1.20 (d, 6H, J=6.8 Hz, —CH₃), 1.34 (d, 3H, J=6.7 Hz, —CH₃), 1.42-1.73 (m, 6H, —CH₂—), 1.85 (m, 3 H, —CH₂—), 2.86 (m, 1H, J=6.8 Hz, —CH—), 3.21 (m, 2H, J=6.9 Hz, —CH—), 3.27 (m, 1 H, —CH—), 4.93 (tt, 1H, J=11.4, 3.7 Hz, —CH—), 7.15 (dd, 1H, J=7.7, 1.3 Hz, ArH), 7.18 (d, 2H, J=7.9 Hz, ArH), 7.28 (dd, 1H, J=7.8, 1.4 Hz, ArH), 7.39 (td, 2H, J=7.8, 2.1 Hz, ArH); ¹³C NMR (75 MHz, CD₂Cl₂): δ=20.65, 21.17, 23.49, 23.63, 23.87, 24.51, 27.22, 27.26, 29.29, 29.54, 30.36, 43.69, 53.03, 124.73, 125.01, 126.19, 130.76, 131.80, 134.57, 146.85, 146.96, 149.31, 185.97; IR (KBr): ν=2963, 2931, 2867, 2188, 1589, 1500, 1458, 1386, 1365, 1336, 1304, 1269, 1184, 1111, 1055, 804, 750, 731, 626; HR-MS (FAB): calc.: C₃₄H₄₉ClN₃Pd [M−Cl⁻]⁺=640.2650. found: 640.2672

### Example 8

¹H NMR (300 MHz, CDCl₃): δ=1.0 (d, 3H, J=6.8 Hz, —CH₃), 1.02 (d, 3H, J=6.9 Hz, —CH₃), 1.07 (d, 3H, J=6.9 Hz, —CH₃), 1.17 (d, 6H, J=6.9 Hz, —CH₃), 1.19 (d, 6H, J=6.9 Hz, —CH₃), 1.4 (d, 3H, J=6.4 Hz, —CH₃), 1.69 (m, 6H, —CH₂—), 2.23 (m, 3H, —CH—), 2.43 (m, 3H, —CH—, —CH₂—), 2.74 (m, 3H, —CH—, —CH₂—), 2.86 (m, 1H, J=6.8 Hz, —CH—), 3.24 (m, 2H, J=6.8 Hz, —CH—), 3.36 (m, 1H, —CH—), 3.66-4.01 (m, 4H, —CH₂—), 7.06 (dd, 1H, J=7.7, 1.6 Hz, ArH), 7.12 (d, 2H, J=7.8 Hz, ArH), 7.25 (dd, 1H, J=7.8, 1.8 Hz, ArH), 7.33 (td, 2H, J=7.9, 2.8 Hz, ArH); ¹³C NMR (75 MHz, CDCl₃): δ=23.09, 23.33, 23.57, 24.14, 27.01, 27.10, 28.94, 29.16, 29.75, 30.02, 36.12, 42.96, 45.90, 52.74, 59.42, 124.04, 124.06, 125.93, 130.17, 131.02, 135.49, 145.74, 146.33, 148.95, 184.88; IR (KBr): ν=2965, 2911, 2869, 2186, 1630, 1478, 1456, 1386, 1363, 1330, 1305, 1256, 1190, 1100, 1056, 804, 751, 620; HR-MS (FAB⁺): calc.: C₃₈H₅₃ClN₃Pd [M−Cl⁻]⁺=692.2963. found: 692.2974

### Example 9

(Mixture of diastereoisomers); ¹H NMR (300 MHz, CD₂Cl₂): δ=−0.14 (d, J=6.6 Hz, —CH₃), −0.06 (d, J=6.8 Hz, —CH₃), 0.79 (d, J=6.8 Hz, —CH₃), 1.06-1.99 (m, 29H), 1.56-3.11 (m, 3H, —CH₂—), 3.44 (m, J=6.6 Hz, —CH—), 3.87 (dd, J=11.4, 4.0 Hz, —CH₂—), 4.01 (m, —CH₂—), 4.36 (m, —CH₂—), 4.88 (dd, J=11.2, 4.0 Hz, —CH—), 5.11 (m, —CH—), 6.97-7.45 (m, 11H, ArH); ¹³C NMR (75 MHz, CD₂Cl₂): δ=22.46, 22.80, 23.54, 23.62, 23.85, 24.51, 24.73, 25.00, 25.50, 25.58, 25.77, 26.49, 26.79, 26.90, 28.87, 28.96, 29.26, 29.42, 30.01, 30.11, 30.25, 30.81, 31.58, 31.79, 46.15, 52.14, 60.20, 60.68, 68.09, 68.79, 123.97, 124.27, 124.44, 124.62, 124.98, 125.88, 126.28, 128.09, 128.27, 129.64, 129.75, 129.85, 131.16, 131.47, 133.04, 134.34, 139.45, 140.07, 145.68, 145.78, 146.94, 147.27, 147.70, 150.06, 184.67, 186.02; IR (KBr): ν=2943, 2858, 2195, 1631, 1512, 1486, 1473, 1443, 1381, 1354, 1307, 1273, 1253, 1168, 1143, 1096, 1011, 779, 632; HR-MS (FAB⁺): calc.: C₄₀H₅₃ClN₃Pd [M−Cl⁻]⁺=716.2963. found: 716.3002

### Example 10

¹H NMR (300 MHz, CDCl₃): δ=1.49 (s, 9H, CH₃), 1.71 (s, 9H, CH₃), 3.57 (s, 3H, CH₃), 3.73 (m, 4H, CH₂); ¹³C NMR (75 MHz, CDCl₃): δ=30.23, 30.72, 39.22, 47.71, 50.57, 53.64, 56.97, 77.65, 183.1; IR (KBr): ν=3444, 2980, 2218, 1630, 1530, 1466, 1371, 1324, 1287, 1200, 1136, 620; HRMS (FAB⁺) C₁₃H₂₄N₃Pd [M−HCl₂]⁺: calc. 328.1011. found: 328.1005

### Example 11

¹H NMR (300 MHz, CD₂Cl₂): δ=1.19 (d, 3H, J=6.8 Hz, —CH₃), 1.26 (d, 3H, J=6.7 Hz, —CH₃), 1.45 (s, 9H, —CH₃), 1.65 (s, 9H, —CH₃), 3.38-3.79 (m, 4H, —CH₂—), 5.56 (m, 1H, J=6.7 Hz, —CH—); ¹³C NMR (75 MHz, CD₂Cl₂): δ=18.55, 19.27, 29.01, 29.44, 40.68, 46.02, 52.17, 55.79; IR (KBr): ν=2979, 2936, 2876, 2217, 1635, 1504, 1454, 1400, 1369, 1322, 1285, 1235, 1197, 1111, 806, 622, 594, 523; HR-MS (FAB⁺): calc.: C₅H₂₉ClN₃Pd [M−Cl⁻]⁺=392.1085. found: 392.1074

### Example 12

¹H NMR (300 MHz, CD₂Cl₂): δ=0.98-2.12 (m, 10H, —CH₂—), 1.45 (s, 9H, —CH₃), 1.66 (s, 9H, —CH₃), 3.39-3.77 (m, 4H, —CH₂—), 5.06 (m, 1H, —CH—); ¹³C NMR (75 MHz, CD₂Cl₂): δ=24.42, 24.53, 29.00, 29.14, 29.45, 30.07, 41.97, 45.97, 55.83, 59.82, 179.92; IR (KBr): ν=2918, 2933, 2856, 2216, 1631, 1502, 1451, 1371, 1313, 1283, 1263, 1247, 1225, 1195, 1088, 1031, 804, 622; HR-MS (FAB⁺): calc.: C₁₈H₃₃ClN₃Pd [M−Cl⁻]⁺=432.1398. found: 432.1410

### Example 13

¹H NMR (300 MHz, CDCl₃): δ=2.35 (s, 3H, CH₃), 2.58 (s, 3H, CH₃), 3.66 (s, 3H, CH₃), 4.02 (m, 4H, CH₂), 7.14 (m, 1H, ArH), 7.19 (m, 1H, ArH), 7.27 (m, 2H, ArH), 7.30 (m, 2H, ArH) ppm; ¹³C NMR (75 MHz, CDCl₃): δ=19.37, 19.75, 38.20, 50.94, 52.10, 77.45, 127.56, 127.74, 129.31, 130.62, 130.99, 131.03, 131.13, 133.06, 135.22, 138.96, 141.41, 186.60; IR (KBr): ν=3431, 2202, 1632, 1549, 1487, 1458, 1413, 1319, 1273, 1114, 781, 730, 617; HRMS (FAB⁺) C₁₉H₁₉N₃Cl₃Pd [M−Cl⁻]⁺: calc. 499.9679. found: 499.9636

### Example 14

(Mixture of diastereoisomers); ¹H NMR (500 MHz, CDCl₃): δ=1.45-2.02 (m; 8H, CH₂), 2.01 (s, —CH₃), 2.03 (s, —CH₃), 2.27 (s, —CH₃), 2.29 (s, —CH₃), 2.52 (s, —CH₃), 2.54 (s, —CH₃), 3.32 (td, J=12.7, 3.3 Hz, —CH₂—), 5.07 (m, —CH₂—), 5.14 (m, —CH₂—), 6.85-6.91 (m, ArH), 7.08-7.10 (m, ArH), 7.16-7.26 (m, ArH); IR (KBr): ν=2939, 2856, 2196, 1631, 1519, 1443, 1381, 1307, 1273, 1254, 782, 631, 577; HRMS (FAB⁺) C₂₄H₂₉N₃ClPd [M−HCl]⁺: calc. 500.1085. found: 500.1060

### Example 15

(Mixture of diastereoisomers, major isomer); ¹H NMR (500 MHz, CDCl₃): δ=0.00 (d, J=6.6 Hz, 3H, —CH₃), 0.88 (d, J=6.6 Hz, 3H, —CH₃), 1.09 (d, J=6.6 Hz, 6H, —CH₃), 1.09 (d, J=6.6 Hz, 6H, —CH₃), 1.26-1.73 (m, 25H, —CH₂—), 1.59 (d, J=6.6 Hz, 6H, —CH₃), 1.68 (m, 2H, —CH₂—), 1.97 (m, 1H, —CH₂—), 2.22 (m, 1H, —CH₂—), 2.59 (m, J=6.6 Hz, 2H, —CH—), 2.84 (m, J=6.6 Hz, 1H, —CH—), 3.54 (m, 1H, —CH—), 3.83 (m, 1H, —CH₂—), 4.29 (m, 1H, —CH₂—), 4.83 (m, 1H, —CH—), 5.24 (m, 1H, —CH—), 6.99 (d, J=7.7 Hz, 1H, ArH), 7.05 (d, J=7.7 Hz, 2H, ArH), 7.17 (d, J=7.7 Hz, 1H, ArH), 7.25-7.33 (m, 5H, ArH), 7.37 (d, J=6.8 Hz, 2H, ArH); ¹³C NMR (125 MHz, CDCl₃): δ=22.83 (2C), 23.09 (2C), 23.67, 24.60, 25.36 (2C), 26.52, 26.58, 26.72 (2C), 26.94 (2C), 27.07, 27.08, 27.11, 27.22, 27.45 (2C), 28.71, 28.80, 29.65 (2C), 30.83, 32.50, 52.72, 60.84, 68.75, 123.50 (2C), 124.11, 126.06, 128.04 (2C), 129.54, 129.56 (2C), 129.61, 130.74, 134.22, 139.64, 145.34, 145.51 (2C), 140.34, 186.39.

### II.1.2 Synthesis NHC-Au(I) Complexes

### General Procedure for the Synthesis of NHC-Au(I) Complexes

In a typical protocol the (isonitrile)-Au(I) complex (132 μmol) and the 2-(chloroethyl)ammonium chloride (396 μmol) were suspended in absolute DCM. Triethylamine (0.5 ml, 6.81 mmol) was added. The mixture was stirred for 96 h at ambient temperature. After this all the volatiles were removed under reduced pressure and the solid was dissolved in DCM (10 ml). The solution was extracted with a saturated solution of NH₄Cl (20 ml). The organic layer was dried with Na₂SO₄ and the solvent was removed under reduced pressure. The crude products were purified by column chromatography (SiO₂, mixtures of petrol ether and ethyl acetate; examples 15 and 18: petrolether/ethyl acetate, 2/1; example 16: petrolether/ethyl acetate, 5/1 and example 17: petrolether/ethyl acetate, 1/1). All the complexes of examples 15 to 18 listed below in table II are air and moisture stable compounds and can be stored at ambient temperature without decomposition.

TABLE II
(I-A.2.1) with M being AuCl

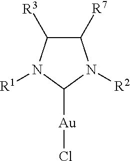

| Ex. | R¹ | R³ | R² | R⁷ | yield/material property |
|---|---|---|---|---|---|
| 16 | 2,4,6-trimethylphenyl | H | isopropyl | H | 45%, colourless solid |
| 17 | 2,4,6-trimethylphenyl | H | 1-adamantyl | H | 50%, colourless solid |
| 18 | 2,6- | H | isopropyl | H | 80%, colourless solid |
| | diisopropylphenyl | | | | |
| 19 | tert-butyl | H | isopropyl | H | 80%, colourless solid |

The physicochemical data of the complexes of the examples 16 to 19 are listed below:

### Example 16

R^{f} (petrol ether/ethyl acetate 2/1)=0.28; ¹H NMR (300 MHz, CD₂Cl₂): δ=1.28 (d, 6 H, J=6.8 Hz, —CH₃), 2.17 (s, 6H, —CH₃), 1.26 (s, 3H, —CH₃), 3.72 (s, 4H, —CH₂—), 4.79 (m, 1H, J=6.8 Hz, —CH—), 6.92 (s, 2H, ArH); ¹³C NMR (75 MHz, CD₂Cl₂): δ=17.01, 19.63, 20.09, 42.34, 49.34, 50.89, 128.68, 134.64, 135.19, 138.03, 191.54; IR (KBr): ν=2968, 1609, 1506, 1456, 1369, 1344, 1322, 1276, 1263, 1202, 1161, 1099, 1058, 1021, 862, 804, 613, 604, 582; HR-MS (FAB⁺): calc.: C₁₅H₂₃ClN₂Au [M+H⁺]⁺=463.1215. found: 463.1194

### Example 17

R^{f} (petrol ether/ethyl acetate 5/1)=0.22; ¹H NMR (500 MHz, CD₂Cl₂): δ=1.69 (s, 6 H), 2.16 (s, 9H), 2.26 (s, 3H), 2.38 (s, 6H), 3.60 (m, 2H, —CH₂—), 3.89 (m, 2H, —CH₂—), 6.92 (s, 2H, ArH); ¹³C NMR (75 MHz, CD₂Cl₂): δ=18.17, 21.27, 30.42, 36.37, 43.75, 46.86, 49.30, 57.43, 129.79, 136.12, 137.20, 139.01, 192.76; IR (KBr): ν=2909, 2851, 1631, 1543, 1494, 1449, 1360, 1312, 1272, 1192, 1142, 1103, 1036, 852, 817, 674, 607, 581

### Example 18

¹H NMR (300 MHz, CD₂Cl₂): δ=1.19 (d, 6H, J=7.0 Hz, —CH₃), 1.29 (d, 12H, J=6.8 Hz, —CH₃), 2.86 (m, 2H, J=6.9 Hz, —CH—), 3.74 (s, 4H, —CH₂—), 4.81 (m, 1H, J=6.8 Hz, —CH—), 7.19 (d, 2H, J=7.8 Hz, ArH), 7.38 (m, 1H, ArH); ¹³C NMR (75 MHz, CD₂Cl₂): δ=19.67, 23.31, 23.91, 27.72, 42.34, 50.92, 51.98, 123.74. 128.89, 134.09, 146.23, 192.18; ¹⁵N NMR (600 MHz, CD₂Cl₂, urea): δ=130.29, 146.50; IR (KBr): ν=2964, 2926, 2867, 1631, 1502, 1460, 1385, 1367, 1318, 1274, 1236, 1192, 1163, 1111, 1059, 1019, 808, 763, 601; HR-MS (FAB⁺): calc.: C₁₈H₂₉ClN₂Au [M+H⁺]⁺=505.1685. found: 505.1652

### Example 19

R^{f} (petrol ether/ethyl acetate//2/1)=0.18; ¹H NMR (300 MHz, CD₂Cl₂): δ=1.19 (d, 6 H, J=6.8 Hz; —CH₃), 1.57 (s, 9H, —CH₃), 3.4 (dd, J=11.4, 8.9 Hz, —CH₂), 3.64 (dd, J=11.4, 8.9 Hz, —CH₂), 4.88 (m, J=6.8 Hz, —CH—); ¹³C NMR (125 MHz, CD₂Cl₂): δ=20.61, 30.92, 41.92, 47.40, 53.65, 56.06, 191.32; IR (KBr): ν=2968, 2875, 1494, 1450, 1397, 1366, 1328, 1282, 1235, 1202, 1117, 955, 711, 676, 610, 521, 436; HR-MS (FAB⁺): calc.: C₁₀H₂₁ClN₂Au [M+H⁺]⁺=401.1059. found: 401.1005

### II.1.3 General Procedure for the Synthesis of NHC-Pt(II) Complexes

In a typical protocol the cis-(isonitrile)-Pt(II) complex (156 μmol) and the 2-(chloroethyl)ammonium chloride (200 μmol) were suspended in absolute DCM. Triethylamine (0.25 ml, 3.4 mmol) was added. The mixture was stirred for 72 h at ambient temperature. After this all the volatiles were removed under reduced pressure and the solid was dissolved in DCM (10 ml). The solution was extracted with a saturated solution of NH₄Cl (20 ml). The organic layer was dried with Na₂SO₄ and the solvent was removed under reduced pressure. All the complexes of examples 19 and 20 listed below in table III are air and moisture stable compounds and can be stored at ambient temperature without decomposition.

TABLE III
(I-A.2.1) with M being PtC1₂(CNR¹)

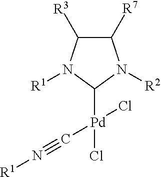

| Ex. | R¹ | R³ | R² | R⁷ | yield/material property |
|---|---|---|---|---|---|
| 20 | 2,6-diisopropylphenyl | H | isopropyl | H | 63%, colourless solid |
| 21 | 2,6-diisopropylphenyl | H | cyclohexyl | H | 70%, colourless solid |

The physicochemical data of the complexes of the examples 20 and 21 are listed below:

### Example 20

¹H NMR (500 MHz, CD₂Cl₂): δ=1.04 (d, 3H, J=6.7 Hz, —CH₃), 1.06 (d, 3H, J=6.7 Hz, —CH₃), 1.13 (d, 3H, J=6.7 Hz, —CH₃), 1.19 (d, 6H, J=6.9 Hz, —CH₃), 1.2 (d, 6H, J=6.9 Hz, —CH₃), 1.34 (d, 6H, J=6.6 Hz, —CH₃), 1.43 (d, 3H, J=6.7 Hz, —CH₃), 2.88 (m, 1H, J=6.7 Hz, —CH—), 3.24 (m, 3H, —CH—), 3.72-3.98 (m, 4H, —CH₂—), 5.50 (m, 1 H, J=6.6 Hz, —CH—), 7.16 (m, 3H, ArH), 7.27 (d, 1H, J=7.6 Hz, ArH), 7.39 (m, 2H, ArH); ¹³C NMR (125 MHz, CD₂Cl₂): δ=20.24, 20.58, 23.03, 23.11, 23.47, 23.93, 26.74, 26.88, 28.89, 29.23, 29.92, 43.24, 52.24, 124.23, 124.60, 125.63, 130.19, 130.68, 134.68, 146.15, 146.47, 148.62, 173.14; ¹⁹⁵Pt NMR (498 MHz, CD₂Cl₂, Na₂PtCl₄): δ=−3669.80; HR-MS (FAB): calc.: C₃₁H₄₅Cl₂N₃Pt [M−Cl⁻]⁺=689.2950. found: 689.2914.

### Example 21

¹H NMR (600 MHz, CD₂Cl₂): δ=1.08 (d, 3H, J=6.8 Hz, —CH₃), 1.10 (d, 3H, J=6.8 Hz, —CH₃), 1.17 (d, 3H, J=6.8 Hz, —CH₃), 1.23 (d, 6H, J=6.8 Hz, —CH₃), 1.25 (d, 6H, J=6.8 Hz, —CH₃), 1.36 (t, 1H, J=7.3 Hz, —CH₂—), 1.38 (d, 3H, J=6.8 Hz, —CH₃), 1.46-1.58 (m, 3H, —CH₂—), 1.67 (qd, 1H, J=12.1, 3.2 Hz, CH₃), 1.74 (d, 1H, J=14.3 Hz, —CH₂—), 1.91 (t, 3H, J=14.3 Hz, —CH₂—), 2.34 (m, 1H, —CH₂—), 2.93 (m, 1H, J=6.8 Hz, —CH—), 3.26 (m, 2H, J=6.8 Hz, —CH—), 3.32 (m, 1H, J=6.8 Hz, —CH—), 3.79-4.03 (m, 4 H, —CH₂—), 5.00-5.06 (m, 1H, —CH—), 7.20 (dd, 1H, J=7.8, 1.4 Hz, ArH), 7.22 (d, 2H, J=7.8, ArH), 7.32 (dd, 1H, J=7.8, 1.4 Hz, ArH), 7.43 (t, 1H, J=7.8, ArH), 7.44 (t, 1 H, J=7.8, ArH); ¹³C NMR (150 MHz, CD₂Cl₂): δ=22.81, 22.92, 23.38, 23.75, 25.41, 25.50, 25.68, 26.57, 26.70, 28.70, 29.05, 29.79, 30.55, 31.36, 44.35, 45.99, 59.68, 124.06, 124.30, 124.41, 125.44, 129.98, 130.49, 134.59, 145.93, 146.29, 148.44, 172.84; HR-MS (FAB⁺): calc.: C₃₄H₄₉Cl₂N₃Pt [M−Cl⁻]⁺=729.3263. found: 729.3265.

### II.2. Synthesis of NHC-(Transition Metal) Complexes of the Formula I-B.2.1

### II.2.1 Synthesis of Au(I)-NHC Complexes

### General Procedure:

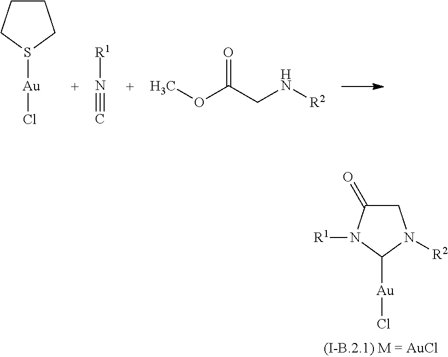

Under an atmosphere of nitrogen (tetrahydrothiophene)AuCl (1.00 equivalent, 50.0 mmol) was dissolved in absolute DCM (0.10 M) at ambient temperature. The isonitrile (1.05 equivalents) in question was added and the solution was stirred for 5 min. After this, the amine in question (1.50 equivalents) was added and stirring was continued for 36 h. The solvent was removed under reduced pressure and the resulting crude product was washed with cold pentane (five times, 2 ml. The product was dried under reduced pressure. Alternatively, the compounds can be purified by column chromatography using mixtures of petrol ether and ethyl acetate on basic alumina.

### Example 22

### Chloro(1-cyclohexyl-3-(2,6-diisopropylphenyl)imidazolin-4-on-2-ylidene)gold(I)

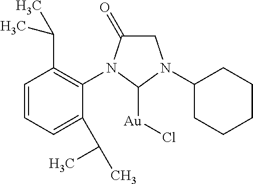

¹H NMR (500 MHz, CDCl₃/C₆D₆/4/1): δ=1.08 (d, J=6.9 Hz, 6H, —CH₃), 1.24 (d, J=6.9 Hz, 6H, —CH₃), 1.42 (m, 5H, —CH₂—), 1.68 (m, 1H, —CH₂—), 1.82 (m, 2H, —CH₂—), 1.97 (m, 2H, —CH₂—), 2.51 (m, J=6.9 Hz, 2H, —CH—), 3.94 (s, 2H, —CH₂—, carbene backbone), 4.44 (m, 1H, —CH—), 7.16 (d, J=7.8 Hz, 2H, ArH), 7.37 (t, J=7.8 Hz, 1H, ArH); ¹³C NMR (125 MHz, CDCl₃/C₆D₆ 4/1): δ=24.26, 24.28, 24.97, 25.10, 29.50, 31.99, 48.59, 63.29, 124.73, 129.24, 131.22, 146.29, 171.84, 201.85.

### Example 23

### Chloro(1-cyclododecyl-3-(2,6-diisopropylphenyl)imidiazolin-4-on-2-ylidene)gold(I)

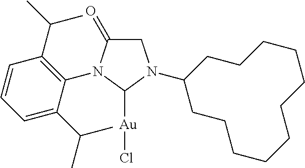

¹H NMR (250 MHz, CDCl₃): δ=1.13 (d, J=6.9 Hz, 6H, —CH₃), 1.21 (d, J=6.9 Hz, 6 H, —CH₃), 1.18-1.71 (m, 20H, —CH₂—), 1.94 (m, 2H, —CH₂—), 2.57 (m, J=6.9 Hz, 2H, —CH—), 4.06 (s, 2H, —CH₂—, carbene backbone), 4.9 (m, 1H, —CH—), 7.22 (d, J=7.7 Hz, 2 H, ArH), 7.44 (t, J=7.7 Hz, 1H, ArH).

### Example 24

### Chloro(3-(2,6-diisopropylphenyl)-1-(1-phenylethyl)imidazolin-4-on-2-ylidene)gold(I)

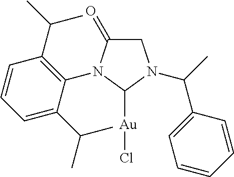

¹H NMR (250 MHz, CDCl₃): δ=1.03 (d, J=6.9 Hz, 3H, —CH₃), 1.10 (d, J=6.9 Hz, 3 H, —CH₃), 1.27 (d, J=6.9 Hz, 2H, —CH₃), 1.29 (d, J=6.9 Hz, 3H, —CH₃), 1.81 (d, J=7.2 Hz, 3H, —CH₃), 2.43 (m, J=6.9 Hz, 2H, —CH—), 3.65 (d, J=21.2 Hz, 1H, —CH₂—, carbene backbone), 4.03 (d, J=21.2 Hz, 1H, —CH₂—, carbene backbone), 6.06 (q, J=7.2 Hz, 3H, —CH—), 7.2 (m, 8H, ArH).

### II.2.2 Synthesis of Pd(II)-NHC Complexes

General procedure for the synthesis of Pd(II)-NHC complexes of the formula I-B.2.1

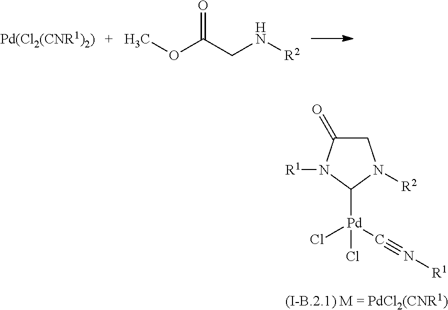

Under an atmosphere of nitrogen the palladium bis(isonitrile) complex in question (1.00 equivalent, 50.0 μmol) was dissolved in absolute THF (0.30 M) at ambient temperature. After this, the amine in question (1.10 equivalents) was added and stirring was continued for 7 d. The solvent was removed under reduced pressure and the resulting crude product was washed with cold pentane (five times, 2.00 ml). The product was dissolved in a minimum of DCM and cold pentane was added to induce precipitation of the Pd(II)-NHC complex.

### Example 25

### Compound of the formula I-B.2.1 with R¹=2,6-diisopropyl, R²=cyclohexyl

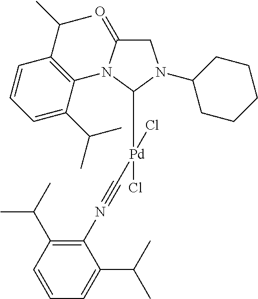

¹H NMR (250 MHz, CDCl₃): δ=0.93 (d, J=6.8 Hz, 3H, —CH₃), 0.96 (d, J=6.8 Hz, 3 H, —CH₃), 1.04 (d, J=6.8 Hz, 3H, —CH₃), 1.19 (d, J=6.8 Hz, 6H, —CH₃), 1.21 (d, J=6.8 Hz, 6H, —CH₃), 1.4 (d, J=6.8 Hz, 3H, —CH₃), 1.10-2.05 (m, 10H, —CH₂—), 2.54 (m, 1 H, —CH—), 3.13 (m, 3H, —CH—), 4.32 (m, 2H, carbene backbone), 5.22 (m, 1H, —CH—), 7.17 (m, 2H, ArH), 7.44 (m, 4H, ArH).

By recrystallization of the title compound from DCM/petrol ether, the compound of the following formula

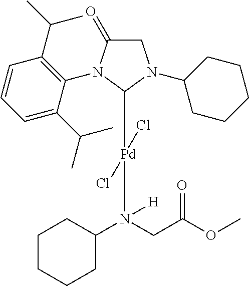

was obtained.

### II.3. Synthesis of NHC-(Transition Metal) Complexes of the Formula I-C.2.1

### II.3.1 General Synthesis of Au(I)-NHC Complexes

Under an atmosphere of nitrogen an chloroimidiazolin-4-on-2-ylidene)gold(I) complex in question (50.0 mg, 77.6 μmol, 1 equivalent) was dissolved in absolute THF. The mixture was cooled to −78° C. and lithium bis(trimethylsilyl)amide (0.14 M in THF, 610 μl, 1.1 equivalents) was added. The mixture was stirred at −78° C. for 30 min prior to adding the electrophile (1.1 equivalents). The mixture was allowed to warm up to ambient temperature and stirring was continued for 30 min. After this, the solvent was removed and the resulting crude product was washed with pentane (three times, 3 ml). The product was dissolved in absolute DCM and filtrated through a pad of celite to remove the LiCl. The solvent was removed under reduced pressure and the resulting solids were stored under an atmosphere of nitrogen at −32° C.

### Example 26

### Chloro(4-((tert-butyldiphenylsilyl)oxy)-1-cyclododecyl-3-(2,6-diisopropylphenyl)-imidazol-2-ylidene)gold(I)

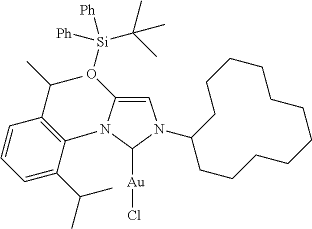

According to the general procedure, the title compound was prepared using tert-butyl diphenylchlorosilane as electrophile.

¹H NMR (250 MHz, CD₂Cl₂): δ=0.85 (s, 9H, —CH₃), 1.09 (d, J=6.9 Hz, 6H, —CH₃), 1.26 (d, J=6.9 Hz, 6H, —CH₃), 0.95-1.79 (m, 22H, —CH₂—), 2.42 (m, J=6.9 Hz, 2 H, —CH—), 4.63 (m, J=6.8 Hz, 1H, —CH—), 5.59 (s, 1H, ═CH), 7.31 (m, 6H, ArH), 7.45 (m, 7H, ArH).

### Example 27

### Chloro(4-(benzoyloxy)-1-cyclododecyl-3-(2,6-diisopropylphenyl)-imidazol-2-ylidene)gold(I)

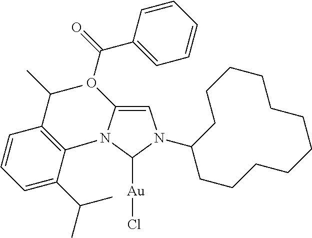

According to the general procedure, the title compound was prepared using benzoyl chloride as electrophile.

¹H NMR (250 MHz, C₆D₆): δ=0.98 (d, J=6.8 Hz, 6H, —CH₃), 1.33 (d, J=6.8 Hz, 6H, —CH₃), 1.05-1.92 (m, 22H, —CH₂—), 2.62 (m, J=6.8 Hz, 2H, —CH—), 5.17 (m, J=6.5 Hz, 1H, —CH—), 6.76 (t, J=7.7 Hz, 2H, ArH), 6.91 (t, J=7.5 Hz, 1H, ArH), 7.03 (d, J=7.7 Hz, 2H, ArH), 7.19 (m, 2H, ArH, ═CH), 7.68 (m, 2H, ArH).

### Example 28

### Chloro(4-((((4,5-dimethoxy-2-nitrobenzyl)oxy)carbonyl)oxy)-1-cyclododecyl-3-(2,6-diisopropylphenyl)-imidazol-2-ylidene)gold(I)

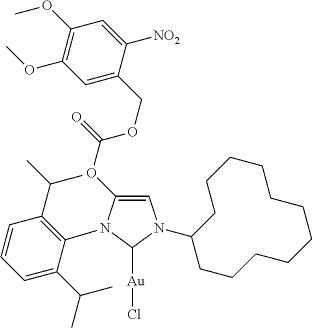

According to the general procedure, the title compound was prepared using 4,5-dimethoxy-2-nitrobenzylchloroformate as electrophile.

¹H NMR (250 MHz, CDCl₃): δ=1.05 (d, J=6.8 Hz, 6H, —CH₃), 1.26 (d, J=6.8 Hz, 6 H, —CH₃), 1.02-1.88 (m, 22H, —CH₂—), 2.32 (m, J=6.8 Hz, 2H, —CH—), 3.83 (s, 3H, —CH₃), 3.93 (s, 3H, —CH₃), 5.58 (s, 2H, —CH₂—), 6.79 (s, 1H, ═CH), 7.16 (s, 1H, ArH), 7.2 (d, J=7.8 Hz, 2H, ArH), 7.44 (t, J=7.8 Hz, 1H, ArH), 7.68 (s, 1H, ArH).

### Iii Catalytic Activity of the Pd(II)-NHC Complexes

### III.1 General Procedure for Suzuki-Miyaura Reactions

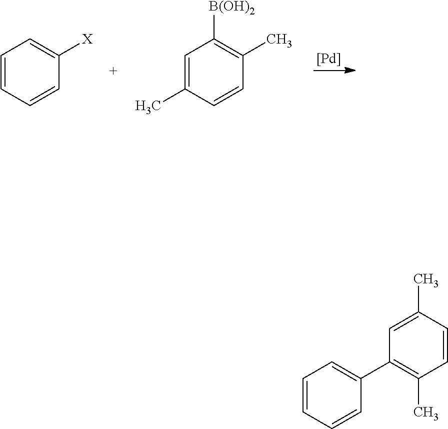

### X═Cl or Br

[Pd]=Pd catalyst of the formula I-A.2.1

There are two protocols for this reaction dependent on the phenyl halide used as substrate.

### Procedure for Chlorobenzene as Substrate

Under an atmosphere of nitrogen, the Pd(II)-NHC complex of example 1, 2, 3, 4, 5, 6, 7, 8, 9, 10, 11, 12, 13 or 14 [1 mol %], 2,5-dimethylphenylboronic acid (1.20 mmol) and chlorobenzene (1.00 mmol) were dissolved in ethanol (2 ml). The mixture was stirred for 5 min and the base potassium tert-butanolate (1.00 mmol) was added. The solution was stirred for 12 h. The yield was determined by GC using dodecane (1.00 mmol) as internal standard. The results are summarized in Table IV.

### Procedure for Bromobenzene as Substrate

Under an atmosphere of nitrogen, the Pd(II)-NHC complex of example 1, 2, 3, 4, 5, 6, 7, 8, 9, 10, 11, 12, 13 or 14 [0.1 mol %], the 2,5-dimethylphenylboronic acid (2.40 mmol) and bromobenzene (2.00 mmol) were dissolved in ethanol (4 ml). The mixture was stirred for 5 min and the base potassium tert-butanolate (2.00 mmol) was added. The solution was stirred for 12 h. The yield was determined by GC using dodecane (1.00 mmol) as internal standard. The results are summarized in table IV.

TABLE IV

| | catalyst | | |
|---|---|---|---|
| | from | Yield (%) starting from | Yield (%) starting from |
| Example | example | chlorobenzene | bromobenzene |
| 29 | 1 | 3 | — |
| 30 | 1 | — | 77 |
| 31 | 2 | 6 | — |
| 32 | 2 | — | 77 |
| 33 | 3 | 3 | — |
| 34 | 3 | — | 84 |
| 35 | 4 | 49 | — |
| 36 | 4 | — | 79 |
| 37 | 5 | 5 | — |
| 38 | 5 | — | 80 |
| 39 | 6 | 40 | — |
| 40 | 6 | — | 79 |
| 41 | 7 | 62 | — |
| 42 | 7 | — | 81 |
| 43 | 8 | 16 | — |
| 44 | 8 | — | 68 |
| 45 | 9 | 64 | — |
| 46 | 9 | — | 83 |
| 47 | 10 | — | 43 |
| 48 | 11 | — | 59 |
| 49 | 12 | — | 53 |
| 50 | 13 | — | 83 |
| 51 | 14 | 4 | — |
| 52 | 14 | — | 83 |

### III.2 General Procedure for the Sonogashira Reaction

Under an atmosphere of nitrogen the catalyst of example 15 (2 mol %) was suspended in ethanol (1.00 ml). Bromobenzene (1.00 mmol, 102 μl) and 1-hexyne (1.50 mmol, 173 μl) were added. Finally, KOtBu (1 mmol, dissolved in 1.00 ml ethanol) was added and the mixture was stirred for 12 h at ambient temperature. The solvent used was technical grade ethanol without prior degassing. After this, ammonium chloride (saturated solution, 2.00 ml), ethyl acetate (4.00 ml) and dodecane (1.00 mmol, 226.2 μl) were added. The organic layer was dried with Na₂SO₄ and analysed by GC. Yield of hex-1-ynylbenzene: 55%.

### IV Synthesis of NHC-(Transition Metal) Complexes of the Formula I-E

### IV.1 Synthesis of Acyclic Gold Complexes of the Formula (VI)

### Route a)

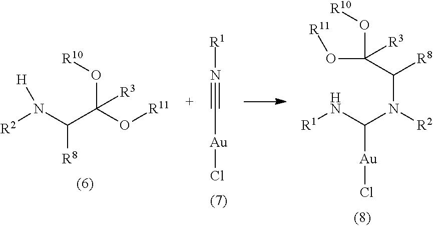

R¹, R², R³, R⁸, R¹⁰ and R¹¹ have the aforementioned meanings.

In a typical protocol, 1-2 equiv. of the amine (6) were added to a solution of an isocyanogold(I) complex (7) in dichloromethane. The mixture was stirred at room temperature for 1-3 days. The solvent was removed under reduced pressure. If necessary, the crude product was purified by column chromatography on silica gel (dichloromethane as eluent).

### Route b)

The synthesis of the acyclic gold(I) carbene (8) can also be achieved in a one-pot procedure, starting from (tetrahydrothiophene)AuCl ((tht)AuCl) in dichloromethane. After addition of the R¹—NC, the mixture was stirred at room temperature for 1 h and the amine (6) was added subsequently.

All the complexes are air and moisture stable and can be stored at room temperature without decomposition.

### Example 53

### Chloro([cyclododecyl(2,2-dimethoxyethyl)amino{[2,6-diisopropylphenyl]amino}methylidene)aurate

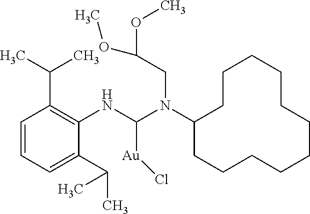

The title compound was prepared according to the general procedure using 100 mg of (238 mmol) 2,6-diisopropylphenylisocyanogold(I) chloride and 71.2 mg (250 mmol) of N-(2,2-dimethoxyethyl)cyclododecanamine in 5 ml dichloromethane. The reaction mixture was stirred at room temperature for 24 h and purified by column chromatography to afford a colourless crystalline solid; yield: 151 mg (219 mmol, 92%); ¹H-NMR (300 MHz, CD₂Cl₂): δ=1.18 (d, J=6.9 Hz, 6H, CH₃), 1.24-1.61 (m, 19H), 1.35 (d, J=6.9 Hz, 6H, CH₃), 3.08 (sept, J=6.9 Hz, 2H, CH), 3.47 (s, 6H, OCH₃), 3.56 (d, J=4.8 Hz, 2H, CH₂N), 4.51 (t, J=4.8 Hz, 1H, CH(OCH₃)₂), 7.21 (d, J=7.4 Hz, 2H, ArH), 7.50 (t, J=7.4 Hz, 1H, ArH); ¹³C-NMR (75 MHz, CD₂Cl₂): δ=22.55 (t, 2C), 22.68 (t, 2C), 23.29 (q, 2C), 23.59 (q, 2C), 24.14 (t, 2C), 24.62 (t, 2C), 24.67 (t, 1C), 28.68 (d, 2C), 29.51 (t, 1C), 48.75 (t, 2C), 53.83 (q, 2C), 64.83 (d, 1C), 105.01 (d, 1C), 124.14 (d, 2H), 129.47 (d, 1C), 135.89 (s, 1C), 146.99 (s, 2C), 198.34 (s, 1C); IR (KBr): ν=3540, 3268, 2931, 2864, 1592, 1537, 1469, 1445, 1414, 1384, 1362, 1331, 1240, 1197, 1162, 1119, 1056, 1016, 801, 759 cm⁻¹; HR-MS (FAB⁺): m/z=690.3202, calcd. for C₂₉H₅₀AuClN₂O₂ [M]⁺: 690.3226, m/z=655.3509. calcd. for C₂₉H₅₀AuN₂O₂ [M−Cl⁻]⁺: 655.3538.

### IV.2 Synthesis of Acyclic-(Palladium or Platin) Complexes

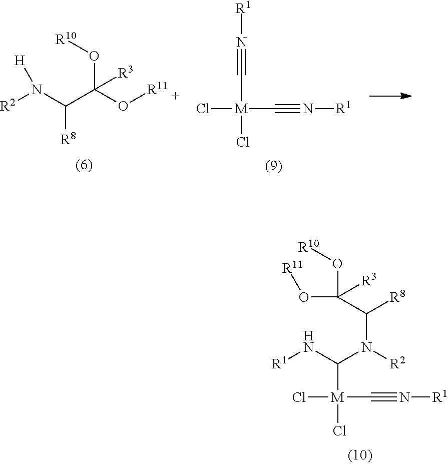

### M is Pd or Pt;

R¹, R², R³, R⁸, R¹⁰ and R¹¹ are as defined above.

In a typical protocol, cis-(isonitrile)-2-MeCl₂ complex (9) and 1 equiv. of the amine (6) were stirred in dry THF under an atmosphere of nitrogen for 2-5 days at room temperature. Complete consumption of the starting material was monitored by the shift of the IR stretching frequencies of the isonitrile ligands. The solvent was removed under reduced pressure and the crude product was dissolved in a minimum amount of dichloromethane and covered with a layer diethyl ether, inducing crystallization of the acyclic carbene complex (10) as colourless crystals. The crystals were filtered off, washed with n-pentane or diethyl ether and dried under reduced pressure. The complexes are air and moisture stable and can be stored at room temperature without decomposition.

### Example 54

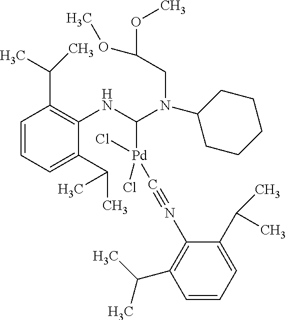

The title compound was prepared according to the typical protocol above using 70 mg of (0.31 mmol) cis-[PdCl₂(2,6-diisopropylphenyl isonitrile)₂ and 58.1 mg (0.31 mmol) of N-(2,2-dimethoxyethyl)cyclohexanamine in 8 ml dry of THF. Yield: 204 mg (276 mmol, 89%); ¹H-NMR (500 MHz, CD₂Cl₂): δ=0.93 (d, J=6.9 Hz, 3H, CH₃), 1.08 (d, J=6.9 Hz, 3H, CH₃), 1.13 (d, J=6.9 Hz, 3H, CH₃), 1.23 (d, J=6.9 Hz, 6H, CH₃), 1.24 (d, J=6.9 Hz, 6H, CH₃), 1.52 (d, J=6.9 Hz, 3H, CH₃), 1.52-1.72 (m, 6H), 1.82 (m, 1H), 1.96 (m, 3H), 2.45 (m, 1H), 2.87 (sept, J=6.9 Hz, 1H, CH), 3.06 (sept, J=6.9 Hz, 2H, CH), 3.51 (s, 3H, CH₃), 3.60 (s, 3H, CH₃), 3.71 (d, J=4.7 Hz, 2H, CH₂), 3.88 (sept, J=6.9 Hz, 1H, CH), 4.57 (t, J=4.7 Hz, 1H, CH), 7.11 (m, 1H, ArH), 7.23 (m, 2H, ArH), 7.44 (m, 3H, ArH), 8.71 (bs, 1H, NH); ¹³C-NMR (500 MHz, CD₂Cl₂): 20.88, 22.56, 22.97 (2C), 23.35 (2C), 25.63, 25.70, 25.88 (2C), 26.04 (2C), 26.08 28.84, 29.72, 29.98 (2C), 31.36, 31.60, 50.17, 55.34, 57.01, 68.37, 105.59, 123.05, 124.1, 125.1, 129.77, 131.18, 134.65, 145.85, 146.15 (2C), 149.38, 191.16; IR (KBr): ν=3447, 3262, 2964, 2932, 2863, 2185, 1665, 1630, 1591, 1543, 1463, 1418, 1386, 1360, 1331, 1142, 1119, 1060, 800, 750 cm⁻¹; HR-MS (FAB⁺): m/z=702.3038, calcd. for C₃₆H₅₅ClN₃O₂Pd [M−Cl⁻]⁺: 702.3028, m/z=667.3921, calcd. for C₃₆H₅₅N₃O₂Pd [M−2Cl⁻]⁺: 667.3329.

### IV.3 Synthesis of Complexes of the Formula I-E

In a typical protocol, the acyclic Au(I)-complex (8) and Pd(II)- or Pt(II) complex (10), respectively, were dissolved in dichloromethane. HCl (4N in HCl) was added and the mixture was stirred at room temperature for 12 h. The solvent was removed under reduced pressure. If necessary, the crude product was purified by column chromatography on silica gel (dichloromethane as eluent) to afford the NHC complexes as crystalline solids in quantitative yields. All the complexes are air and moisture stable and can be stored at room temperature without decomposition.

### Example 55

### Chloro{1-cyclooctyl-3-[2,6-diisopropylyl)phenyl]-1,3-dihydro-2H-imidazol-2-ylid-ene}aurate

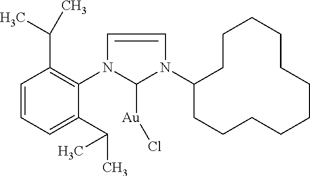

100 mg (0.16 mmol) of chloro([cyclooctyl(2,2-di-methoxyethy)amino{[2,6-diisopropylphenyl]amino}methyl-idene)aurate and 0.25 ml of HCl (4N in dioxane) in 5 ml of dichloromethane were stirred at room temperature for 12 h. Evaporation of the solvent afforded the title compound as a colourless crystalline solid; yield: 90.0 mg (0.16 mmol, >99%); ¹H NMR (300 MHz, CD₂Cl₂): δ=1.12 (d, J=6.9 Hz, 6H), 1.28 (d, J=6.9 Hz, 6H), 1.38-1.96 (m, 10H), 2.02-2.18 (m, 4H), 2.33 (sept, J=6.9 Hz, 2H), 5.01 (quin, J=6.9 Hz, 1H), 6.95 (d, J=1.9 Hz, 1H), 7.24 (d, J=1.9 Hz, 1H), 7.31 (d, J=7.8 Hz, 2H), 7.49 (t, J=7.8 Hz, 1H); ¹³C NMR (75 MHz, CD₂Cl₂): δ=24.54 (q, 2C), 24.62 (q, 2C), 24.91 (t, 2C), 26.35 (t, 1C), 27.22 (t, 2C), 28.97 (d, 2C), 34.78 (t, 2C), 62.72 (d, 1C), 118.27 (d, 1C), 123.94 (d, 1C), 124.66 (d, 2C), 130.91 (d, 1C), 146.48 (s, 2C), 147.07 (s, 1C), 172 (s, 1C); IR (KBr): ν=3433, 2961, 2925, 1865, 1544, 1471, 1459, 1423, 1411, 1385, 1364, 1307, 1254, 1194, 1120, 1058, 873, 807, 765, 740; HR-MS (FAB): m/z=570.2086, calcd. for C₂₃H₃₄AuClN₂ [M]⁺: 570.2076.

IV.4 Synthesis of Compounds of the Formula I-F with M being Au—Cl

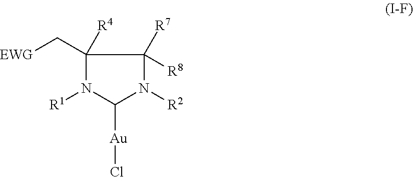

R¹, R², R⁴, R⁷, R⁸ and EWG have one of the meanings given above.

Method A: Under an atmosphere of nitrogen the [AuCl(isonitrile)] (1.00 equivalent) complex was dissolved in absolute DCM and the amine (1.50 equivalents) was added. The mixture was stirred for 48 h and the solvent was removed under reduced pressure. The resulting solid was washed with petrol ether to remove the residual amine. Alternatively, the crude products can be purified by column chromatography using silica and mixtures of petrol ether/ethyl acetate (5:1).

Method B: Under an atmosphere of nitrogen the [AuCl(tht)] (1.00 equivalent) (tht=tetrahydrothiophene) was dissolved in absolute DCM and the isonitrile (1.00 equivalent) was added. The mixture was stirred for 5 min at RT and the amine (1.50 equivalents) was added. The mixture was stirred for 48 h at RT and the solvent was removed under reduced pressure. The resulting solid was washed with petrol ether to remove the residual amine. Alternatively, the crude products can be purified by column chromatography using silica and mixtures of petrol ether/ethyl acetate (5:1).

### Example 56

### (1-Cyclododecyl-3-(2,6-diisopropylphenyl)-4-(2-methoxy-2-oxoethyl)-imidazolidin-2-ylidene)gold(I) chloride

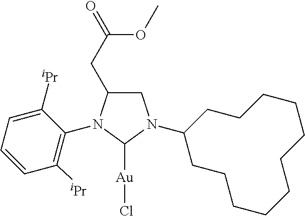

Colourless solid, 80.0 mg, 114 μmol, 38%;

¹H NMR (301 MHz, CD₂Cl₂) δ=1.17 (d, J=6.8 Hz, 3H, —CH₃), 1.19 (d, J=6.8 Hz, 3 H, —CH₃), 1.21 (d, J=6.8 Hz, 3H, —CH₃), 1.30 (d, J=6.8 Hz, 3H, —CH₃), 1.14-1.84 (m, 22H, —CH₂—), 2.67 (m, J=6.8 Hz, 1H, —CH—), 2.92 (m, J=6.8 Hz, 1H, —CH₃), 3.32 (dd, J=11.5, 10.5 Hz, 1H, —CH₂—), 3.48 (s, 3H, —OCH₃), 3.99 (d, J=11.2 Hz, 1H, —CH₂—), 4.27 (m, 1H, —CH—), 4.63 (m, 1H, —CH—), 7.15 (m, 2H, ArH), 7.34 (t, J=7.7 Hz, 1H, ArH); ¹³C NMR (75 MHz, CD₂Cl₂) δ=22.49 (t), 22.59 (t), 22.95 (t), 23.24 (t), 23.34 (t), 23.46 (q), 24.10 (q), 24.24 (t), 24.41 (t), 24.66 (t), 24.95 (t), 25.47 (q), 26.30 (q), 28.62 (t), 28.84 (d), 29.15 (d), 29.25 (t), 37.93 (t), 50.54 (t), 52.36 (q), 55.92 (d), 61.41 (d), 124.99 (d), 125.36 (d), 130.30 (d), 133.20 (s), 147.83 (s), 148.34 (s), 170.55 (s), 194.45 (s, carbene carbon atom).

### Example 57

### (1-Cyclododecyl-4-(2-methoxy-2-oxoethyl)-3-(2-(trifluoromethyl)phenyl)-imidazolidin-2-ylidene)gold(I) chloride

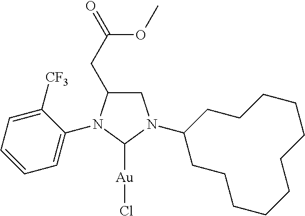

Colourless solid, 115 mg, 167 μmol, 56%; ¹H NMR (300 MHz, CD₂Cl₂) δ=1.06-1.83 (m, 22H, —CH₂—), 2.50 (m, 2H, —CH₂—), 3.49 (m, 1H, —CH₂—), 3.52 (s, 3H, —CH₃), 3.90 (t, J=11.0 Hz, 1H, —CH₂—), 4.55 (m, 1H, —CH—), 4.64 (m, 1H, —CH—), 7.41 (d, J=7.7 Hz, 1H, ArH), 7.54 (m, 1H, ArH), 7.63 (t, J=7.7 Hz, 1H, ArH), 7.72 (d, J=7.7 Hz, 1H, ArH); IR (KBr) ν=2935, 2862, 1738, 1605, 1585, 1500, 1459, 1438, 1316, 1266, 1207, 1174, 1130, 1062, 1037, 999, 773, 645, 599

### Example 58

### (1-Cyclododecyl-4-(2-methoxy-2-oxoethyl)-3-(naphthalen-2-yl)imidazolidin-2-ylidene)gold(I) chloride

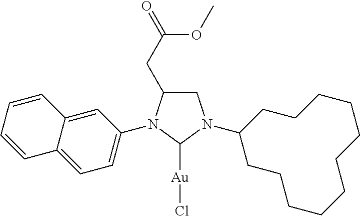

Colourless solid, 149 mg, 223 μmol, 74%; ¹H NMR (300 MHz, CD₂Cl₂) δ=1.10-1.87 (m, 22H, —CH₂—), 2.45 (dd, J=16.5, 9.0 Hz, 1H, —CH₂—), 2.64 (dd, J=16.5, 4.3 Hz, 1H, —CH₂—), 3.48 (m, 1H, —CH₂—), 3.50 (s, 3H, —CH₃), 3.97 (t, J=11.3 Hz, 1H, —CH₂—), 4.71 (m, 1H, —CH—), 4.83 (m, 1H, —CH—), 7.46 (m, 2H, ArH), 7.56 (m, 1H, ArH), 7.82 (m, 4H, ArH).

What is claimed is:

1. A process for preparing compounds of the general formula (I) 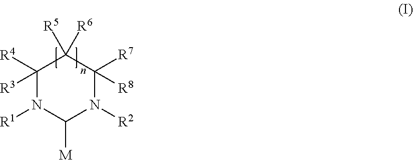
where
n is 0 or 1,
M is a metal atom containing group,
R¹ is selected from hydrogen, alkyl, cycloalkyl, heterocycloalkyl, aryl and hetaryl,
R² is selected from hydrogen, alkyl, cycloalkyl, heterocycloalkyl, aryl and hetaryl,
wherein R¹ and R² do not both stand for hydrogen,
R³ and R⁴ are independently selected from hydrogen and in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
or R³ and R⁴ together with the carbon atom to which they are bound are C═O,
or R³ is a group O—R^{3a} and
for n=0, R⁴ and R⁷ stand for the bond equivalent of a double bond between the carbon atoms carrying R⁴ and R⁷, respectively or
for n=1, R⁴ and R⁵ stand for the bond equivalent of a double bond between the carbon atoms carrying R⁴ and R⁵, respectively, where
R^{3a} is a group bound to the oxygen via a carbon atom, silicon atom, sulfur atom, phosphorus atom, boron atom or titanium atom,
R⁵, R⁶, R⁷ and R⁸ are independently selected from hydrogen and in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
wherein the two radicals R² and R⁸ may also form together with the N atom to which R² is bound a 3- to 12-membered, unsubstituted or substituted nitrogen heterocycle which may optionally have 1, 2 or 3 further heteroatoms or heteroatom containing groups independently selected from O, N, NR^{a} and S as ring members, wherein R^{a} is hydrogen, alkyl, cycloalkyl or aryl,
or
if n=0, R⁴ and R⁷ also may stand for the bond equivalent of a double bond between the carbon atoms carrying R⁴ and R⁷,
the process comprising:
a1) the reaction of an isonitrile complex of the general formula (II)
R¹—N≡C-M (II)
in which R¹ and M have one of the meanings given above,
with a compound of the general formulae (III) or (IIIa) 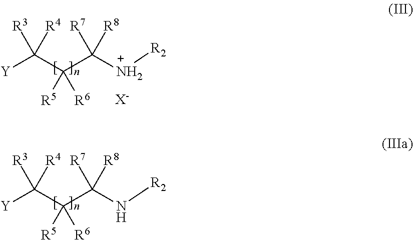
in which n, R², R³, R⁴, R⁵, R⁶, R⁷ and R⁸ have one of the meanings given above,
X⁻ is an anion equivalent, and
Y is a leaving group, or
if R³ and R⁴ together with the carbon atom to which they are bound are C═O then Y is a group O—Y^{a}, where Y^{a} is unsubstituted or substituted alkyl, unsubstituted or substituted aryl, unsubstituted or substituted arylcarbonyl or unsubstituted or substituted alkyl carbonyl, and
b1) optionally, if R³ and R⁴ together with the carbon atom to which they are bound are C═O, subjecting the product obtained in step a1) to a further reaction with a compound R^{3a}—Z, where Z is a leaving group, in the presence of a base to obtain a compound of the formula (I) where R³ is a group O—R^{3a} and for n=0 R⁴ and R⁷ stand for the bond equivalent of a double bond between the carbon atoms bound to R⁴ and R⁷ or for n=1 R⁴ and R⁵ stand for the bond equivalent of a double bond between the carbon atoms bound to R⁴ and R⁵,
or
a2) the reaction of an isonitrile complex of the general formula II
R¹—N≡C-M (II)
where R¹ and M have one of the meanings given above,
with a compound of the general formula (V) 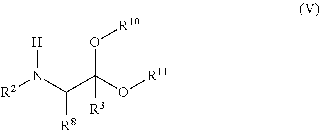
where
R², R³ and R⁸ have one of the meanings given above; and
R¹⁰ and R¹¹ are independently selected from C₁-C₄-alkyl or R¹⁰ and R¹¹ together are linear C₂-C₄-alkylene which may be substituted by one or more C₁-C₄ alkyl radicals;
to give an intermediate compound of the formula (VI) 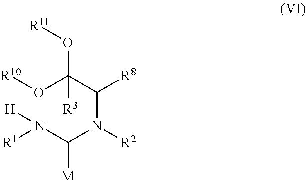
in which R¹, R², R³, R⁸, R¹⁰, R¹¹ and M are as defined above
and
b2) the treatment of the intermediate compound of the formula (VI) with an acid, wherein in compound (I) n is 0, and R⁴ and R⁷ stand for the bond equivalent of a double bond between the carbon atoms carrying R⁴ and R⁷;
or
a3) the reaction of an isonitrile complex of the general formula II
R¹—N≡C-M (II)
where R¹ and M have one of the meanings given above,
with a compound of the general formulae (IIIb) or (IIIc) 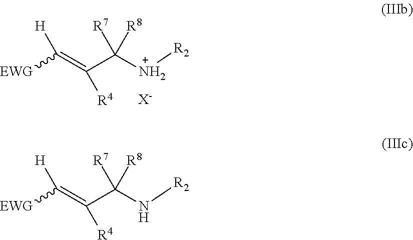
where
R², R⁴, R⁷ and R⁸ have one of the meanings given above;
X⁻ is an anion equivalent; and
EWG is (C(O)R¹⁴, C(O)OR¹⁴, NO₂, S(O)R¹⁴ or S(O)₂R¹⁴, where R¹⁴ is hydrogen, alkyl, cycloalkyl or aryl;
wherein in compound (I) obtained according to variant a3) n is 0 and R³ is CH₂-EWG.

2. The process according to claim 1, wherein the process comprises:
al) the reaction of an isonitrile complex of the general formula (II)
R¹—N≡C-M (II)
in which R¹ and M have one of the meanings given above,
with a compound of the general formulae (III) or (IIIa) 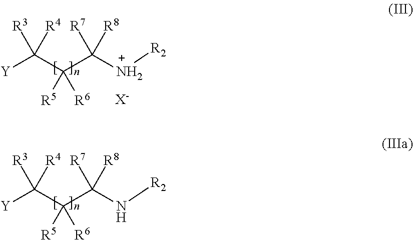
in which
n, R², R³, R⁴, R⁵, R⁶, R⁷ and R⁸ have one of the meanings given above,
X⁻ is an anion equivalent, and
Y is a leaving group, or
if R³ and R⁴ together with the carbon atom to which they are bound are C═O then Y is a group O—Y^{a}, where Y^{a} is unsubstituted or substituted alkyl, unsubstituted or substituted aryl, unsubstituted or substituted arylcarbonyl or unsubstituted or substituted alkyl carbonyl, and
b1) optionally, if R³ and R⁴ together with the carbon atom to which they are bound are C═O, subjecting the product obtained in step a1) to a further reaction with a compound R^{3a}—Z, where Z is a leaving group, in the presence of a base to obtain a compound of the formula (I) where R³ is a group O—R^{3a} and for n=0 R⁴ and R⁷ stand for the bond equivalent of a double bond between the carbon atoms bound to R⁴ and R⁷ or for n=1 R⁴ and R⁵ stand for the bond equivalent of a double bond between the carbon atoms bound to R⁴ and R⁵.

3. The process according to claim 1 wherein R¹ and R² have different meanings.

4. The process according to claim 1, in which M comprises Pd(II), Pt(II) or Au(I).

5. The process according to claim 4, in which M is selected from PdCl₂(CNR¹), PtCl₂(CNR¹), Au(CNR¹) and AuCl, where R¹ is selected from hydrogen, alkyl, cycloalkyl, heterocycloalkyl, aryl and hetaryl.

6. The process according to claim 1, wherein R¹ is selected from groups of the formulae IV.1 to IV.5: 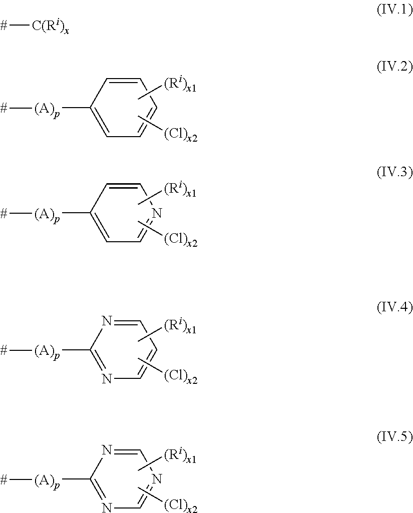
in which
# represents the bonding site to the nitrogen atom,
P is 0 or 1,
x is 2 or 3, where, in the case that x is 2, the carbon atom which bears the Rⁱ radicals additionally bears 1 hydrogen atom,
x₁ in the formulae IV.2, IV.3 and IV.4 is 0, 1, 2 or 3,
x₂ in the formulae IV.2, IV.3 and IV.4 is 0 or 1,
with the proviso that the sum of x₁ and x₂ in the formulae IV.2, IV.3 and IV.4 is 0, 1, 2 or 3,
x₁ in the formula IV.5 is 0, 1 or 2,
x₂ in the formula IV.5 is 0 or 1,
with the proviso that the sum of x₁ and x₂ in the formulae IV.5 is 0, 1 or 2,
A where present, is a C₁-C₁₀-alkylene group which may be interrupted by one or more nonadjacent groups which are selected from —O— and —S—,
the Rⁱ radicals are each independently selected from C₁-C₃₀-alkyl, C₁-C₃₀-alkyloxy or C₁-C₃₀-alkylthio, wherein the alkyl chain in alkyl, alkyloxy or alkylthio may be interrupted by one or more nonadjacent oxygen atom(s).

7. The process according to claim 1, wherein R² is selected from alkyl and cycloalkyl, preferably from methyl, ethyl, n-propyl, isopropyl, n-butyl, sec-butyl, tert-butyl, cyclopentyl, cyclohexyl, cyclododecyl and 1-adamantyl.

8. The process according to claim 1, for preparing compounds of the general formulae (I-A.1) or (I-A.2) 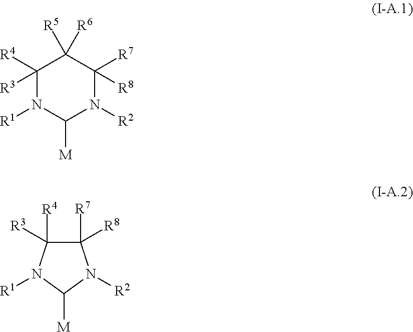
where
M, R¹, R², R⁵, R⁶, R⁷ and R⁸ are as defined in claim 1,
R³ and R⁴ are independently selected from hydrogen and in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
the process comprising:
a1) the reaction of an isonitrile complex of the general formula (II)
R¹—N≡C-M (II)
in which R¹ and M have one of the aforementioned meanings,
with a compound of the general formulae (III) or (IIIa) 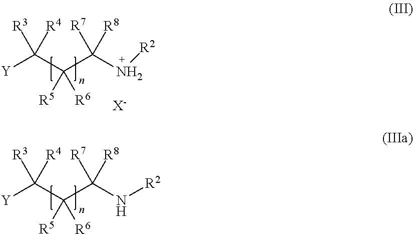
in which
n, R², R³, R⁴, R⁵, R⁶, R⁷ and R⁸ have one of the aforementioned meanings,
X⁻ is an anion equivalent, and
Y is a leaving group.

9. The process according to claim 8 for preparing compounds of the general formula (I-A.2.1) 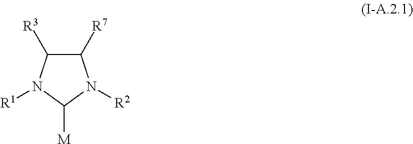
where
M, R¹, R², R³ and R⁷ are as defined in claim 1.

10. The process according to claim 8, where R³, R⁴, R⁷, R⁸, and, if present, R⁵ and R⁶, are all hydrogen.

11. The process according to claim 1, for preparing compounds of the general formula (I-B.1) or (I-B.2) 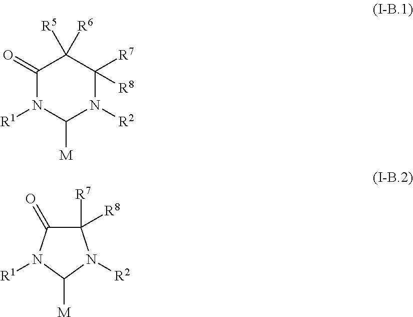
where
M, R¹, R², R⁵, R⁶, R⁷ and R⁸ are as defined in claim 1,
the process comprising:
a1) the reaction of an isonitrile complex of the general formula (II)
R¹—N≡C-M (II)
in which R¹ and M have one of the meanings given above,
with a compound of the general formulae (III-B.1), (III-B.1.a), (III-B.2) or (III-B.2.a) 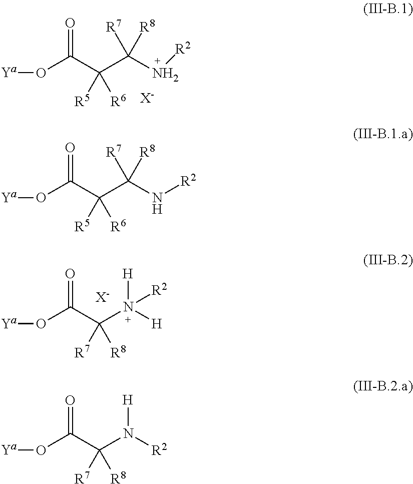
in which
R², R⁵, R⁶, R⁷ and R⁸ have one of the meanings given above,
X⁻ is an anion equivalent, and
Y^{a} is unsubstituted or substituted alkyl, unsubstituted or substituted aryl or unsubstituted or substituted alkyl carbonyl.

12. The process according to claim 11 for preparing compounds of the general formula (I-B.2.1) 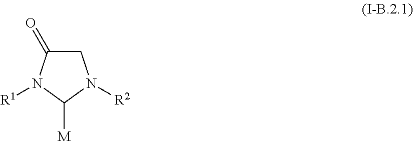
where
M, R¹ and R² are as defined in claim 1.

13. The process according to claim 1, for preparing compounds of the general formula (I-C.1) or (I-C.2) 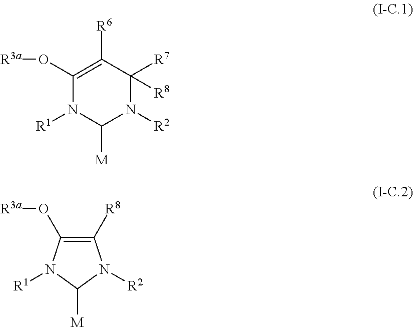
where
M, R¹, R², R^{3a}, R⁶, R⁷ and R⁸ are as defined in claim 1,
the process comprising:
a1) the reaction of an isonitrile complex of the general formula (II)
R¹—N≡C-M (II)
in which R¹ and M have one of the meanings given above,
with a compound of the general formulae (III-C.1) (III-C.1.a), (III-C.2) or (III-C.2.a) 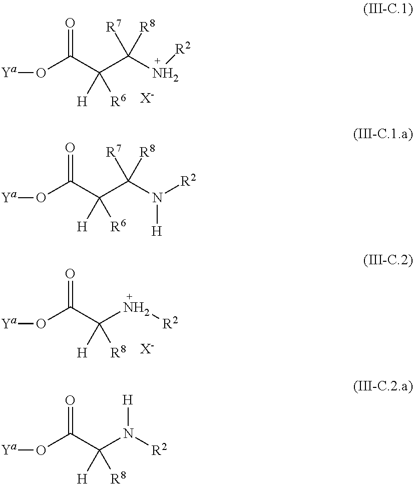
in which
R², R⁶, R⁷ and R⁸ have one of the meanings given above,
X⁻ is an anion equivalent, and
Y^{a} is unsubstituted or substituted alkyl, unsubstituted or substituted aryl or unsubstituted or substituted alkyl carbonyl, and
b1) subjecting the product obtained in step a1) to a further reaction with a compound R^{3a}—Z, where Z is a leaving group, in the presence of a base.

14. The process according to claim 13 for preparing compounds of the general formula (I-C.2.1) 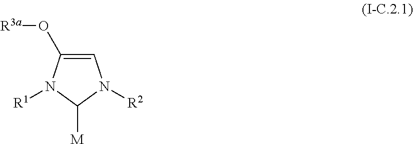
where M, R¹, R² and R^{3a} are as defined in claim 1.

15. The process according to claim 13, wherein R^{3a} is selected from groups of the formulae V-A, V-B, V-C, V-D, V-E, V-F, V-G, V-H, V-I, V-K or V-L 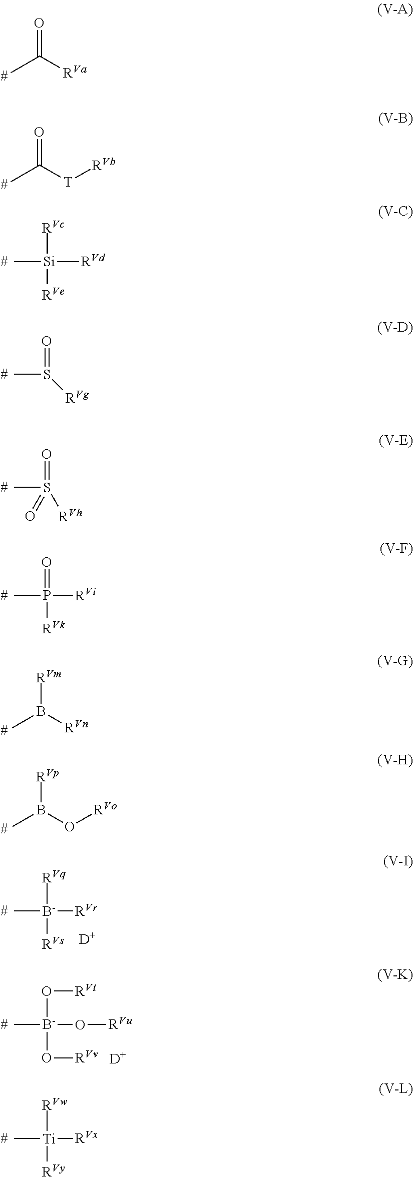
wherein
# represents the bonding site to the oxygen atom,
T is selected from —O— and —NR^{Vf}, wherein R^{Vf} is hydrogen, alkyl, cycloalkyl or aryl,
R^{Va}, R^{Vb}, and R^{Vh} are selected from unsubstituted or substituted alkyl, unsubstituted or substituted cycloalkyl, unsubstituted or substituted aryl and unsubstituted or substituted hetaryl,
R^{Vc}, R^{Vd}, R^{Ve} are independently of each other selected from unsubstituted or substituted alkyl, unsubstituted or substituted cycloalkyl, unsubstituted or substituted aryl and unsubstituted or substituted hetaryl,
R^{Vg} is selected from unsubstituted or substituted heterocycloalkyl,
R^{Vi} and R^{Vk} are independently of each other selected from unsubstituted or substituted alkyl, unsubstituted or substituted cycloalkyl, unsubstituted or substituted aryl, alkoxy unsubstituted or substituted aryloxy and unsubstituted or substituted cycloalkyloxy,
R^{Vm} and R^{Vn}, are independently of each other selected from unsubstituted or substituted alkyl, unsubstituted or substituted alkenyl, unsubstituted or substituted cycloalkyl, unsubstituted or substituted aryl and unsubstituted or substituted hetaryl,
R^{Vo} and R^{Vp}, are independently of each other selected from unsubstituted or substituted alkyl, unsubstituted or substituted alkenyl, unsubstituted or substituted cycloalkyl, unsubstituted or substituted aryl and unsubstituted or substituted hetaryl,
R^{Vq}, R^{Vr} and R^{Vs}, are independently of each other selected from unsubstituted or substituted alkyl, unsubstituted or substituted alkenyl, unsubstituted or substituted cycloalkyl, unsubstituted or substituted aryl and unsubstituted or substituted hetaryl,
R^{Vt}, R^{Vu} and R^{Vv}, are independently of each other selected from unsubstituted or substituted alkyl, unsubstituted or substituted alkenyl, unsubstituted or substituted cycloalkyl, unsubstituted or substituted aryl and unsubstituted or substituted hetaryl,
R^{Vw}, R^{Vx} and R^{Vy} are independently of each other selected from unsubstituted or substituted alkyloxy, unsubstituted or substituted alkenyloxy, unsubstituted or substituted cycloalkyloxy and unsubstituted or substituted aryloxy, and
D⁺ is a cation equivalent.

16. The process according to claim 1, in which the reaction in step a1) is performed in the presence of a base.

17. The process according to claim 1, in which the reaction in step b1) is performed in the presence of a base.

18. The process according to claim 1, for preparing compounds of the general formula (I-E) 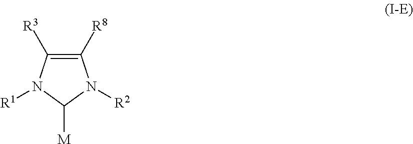
where
M, R¹, R², R³ and R⁸ are as defined in claim 1,
the process comprising:
a2) the reaction of an isonitrile complex of the general formula II
R¹—N≡C-M (II)
where R¹ and M have one of the meanings given above,
with a compound of the general formula (V) 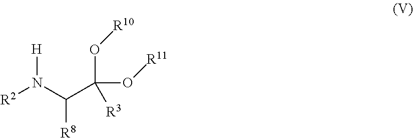
where
R², R³ and R⁸ have one of the meanings given above; and
R¹⁰ and R¹¹ are independently selected from C₁-C₄-alkyl or R¹⁰ and R¹¹ together are linear C₂-C₄-alkylene which may be substituted by one or more C₁-C₄ alkyl radicals;
to give an intermediate compound of the formula (VI) 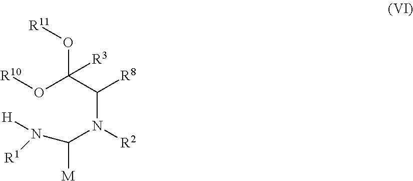
in which R¹, R², R³, R⁸, R¹⁰, R¹¹ and M are as defined above,
and
b2) the treatment of the intermediate compound of the formula (VI) with an acid.

19. The process according to claim 1 for preparing compounds of the general formula (I-F) 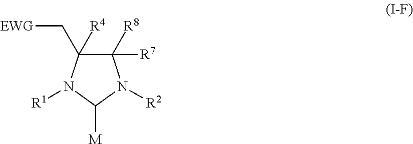
where R¹, R², R⁴, R⁷, R⁸, M and EWG are as defined above,
the process comprising:
a3) the reaction of an isonitrile complex of the general formula II
R¹—N≡C-M (II)
in which R¹ and M are as defined above,
with a compound of the general formulae (IIIb) or (IIIc) 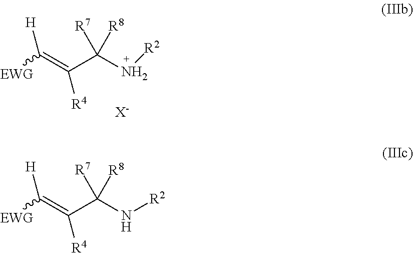
where
X⁻ is an anion equivalent; and
R², R⁴, R⁷, R⁸ and EWG are as defined above.

20. The process according to claim 18, where M comprises AuCl.

21. A compound of the general formula (I), as defined in claim 1.

22. A catalyst comprising the compound of claim 21.

23. A method of forming a C—C, C—O, C—N or C—H bond comprising using the compound of claim 21 as or in a catalyst employed in a C—C, C—O, C—N or C—H bond formation reaction.

24. The method of claim 23, wherein the reaction is a C—C coupling reaction selected from the Suzuki reaction, Heck reaction, Sonogashira reaction, Stille reaction and Kumada reaction.

25. The method of claim 23, wherein the reaction is selected from hydrogenation, hydroformylation, hydrosilylation, the Hartwig-Buchwald reaction and amide α-arylation.

26. A compound of the general formula (VI) 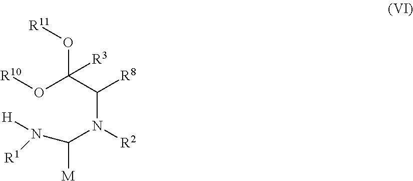
where
R¹ is selected from hydrogen, alkyl, cycloalkyl, heterocycloalkyl, aryl and hetaryl,
R² is selected from hydrogen, alkyl, cycloalkyl, heterocycloalkyl, aryl and hetaryl,
wherein R¹ and R² do not both stand for hydrogen;
R³ is hydrogen and in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino;
R⁸ is hydrogen and in each case unsubstituted or substituted alkyl, alkoxy, alkylthio, (monoalkyl)amino, (dialkyl)amino, cycloalkyl, cycloalkoxy, cycloalkylthio, (monocycloalkyl)amino, (dicycloalkyl)amino, heterocycloalkyl, heterocycloalkoxy, heterocycloalkylthio, (monoheterocycloalkyl)amino, (diheterocycloalkyl)amino, aryl, aryloxy, arylthio, (monoaryl)amino, (diaryl)amino, hetaryl, hetaryloxy, hetarylthio, (monohetaryl)amino and (dihetaryl)amino,
wherein the two radicals R² and R⁸ may also form together with the N atom to which R² is bound a 3- to 12-membered, unsubstituted or substituted nitrogen heterocycle which may optionally have 1, 2 or 3 further heteroatoms or heteroatom containing groups independently selected from O, N, NR^{a} and S as ring members, wherein R^{a} is hydrogen, alkyl, cycloalkyl or aryl
R¹⁰ and R¹¹ are independently selected from C₁-C₄-alkyl or R¹⁰ and R¹¹ together are linear C₂-C₄-alkylene which may be substituted by one or more C₁-C₄ alkyl radicals; and
M is a metal atom containing group.
